(19) United States
(12) Patent Application Publication
(10) Pub. No.: US 2025/0241576 A1
(43) Pub. Date: Jul. 31, 2025

(54) Electromagnetic Transmitters for Continuous Monitoring of Patient Position during Magnetocardiography Scanning

(71) Applicant: SB Technology, Inc., Wilmington, DE (US)

(72) Inventors: Geoffrey Zerbinatti Iwata, San Francisco, CA (US); Madelaine Susan Zoritza Liddy, Mountain View, CA (US); Kevin Robert Tharratt, Redwood City, CA (US); Ethan Jesse Pratt, Santa Clara, CA (US); Christian Thieu Nguyen, Redwood City, CA (US); Stefan Bogdanovic, Mountain View, CA (US)

(21) Appl. No.: 19/035,559

(22) Filed: Jan. 23, 2025

Related U.S. Application Data
(60) Provisional application No. 63/625,871, filed on Jan. 26, 2024.
(60) Provisional application No. 63/625,876, filed on Jan. 26, 2024.

(51) Int. Cl. A61B 5/243; A61B 5/00; A61B 5/0245

(52) U.S. Cl. CPC A61B 5/243; A61B 5/0245; A61B 5/721

(57) ABSTRACT

A method for determining positional information of a target organ of a human subject during a biomagnetic field scan comprises detecting, by a plurality of magnetometers, first biomagnetic field signals from at least a portion of the human subject's organ and second magnetic field signals from an electromagnetic transmitter positioned on a predetermined position of the human subject. The plurality of magnetometers have a known position during the biomagnetic field scan. The method includes determining a time-varying spatial relationship between the electromagnetic transmitter and the plurality of magnetometers based on the detected second magnetic field signals. The method further includes correcting artifacts in the detected first biomagnetic signals in accordance with the determined spatial relationship.

### RELATED APPLICATIONS

This application claims priority to (i) U.S. Provisional Patent Application No. 63/625,871, filed Jan. 26, 2024, titled “Electromagnetic Transmitters for Continuous Monitoring of Patient Position during Magnetocardiography Scanning” and (ii) U.S. Provisional Patent Application No. 63/625,876, filed Jan. 26, 2024, titled “A Combined Electrocardiography and Magnetocardiography System for Continuous Co-Localized Electrophysiology Measurements,” each of which is incorporated by reference herein in its entirety.

This application is related to U.S. patent application Ser. No. ______ (Attorney Docket No. 131075-02-5020-US), filed ___, titled “A Combined Electrocardiography and Magnetocardiography System for Continuous Co-Localized Electrophysiology Measurements,” which is incorporated by reference herein in its entirety.

### TECHNICAL FIELD

The disclosed embodiments relate generally to systems and methods for diagnostic screening via magnetic field sensing.

### BACKGROUND

Measurements of biomagnetic fields in a human subject can be used to understand potential disease states. For example, magnetoencephalography (MEG) is a non-invasive technique for investigating human brain activity. Magnetocardiography (MCG) is a non-invasive technique for measuring magnetic fields produced by electric currents in the heart. Similar to an electrocardiogram, a magnetocardiogram has morphological features such as a QRS complex, P-waves, and T-waves.

### SUMMARY

Magnetocardiography (MCG) and electrocardiography (ECG) are two techniques for measuring electrical activity of the heart. Of the two techniques, MCG has several advantages over electrocardiography (ECG). First, MCG is less impacted by alterations in thoracic conductivity distributions than ECG. Unlike ECGs, in which electric signals can be distorted as they pass through body tissue, magnetic fields are unimpeded by the human body. By measuring more pristine electrical conduction activities in every heartbeat, doctors can better detect abnormal patterns or uncover other indicators about potential disease states. Since MCG is less affected by body conductivity, a sensitive MCG device could reveal subtle functional changes or hypoperfusion that might not appear in an ECG. Second, an MCG device that uses an array of sensors can produce magnetic maps of cardiac activity whereas it is not possible to produce such maps using a standard 12-lead ECG. Third, MCG provides non-contact measurements, which would be valuable in applications where the use of ECG electrodes is not possible, such as in the evaluation of fetuses with life-threatening arrhythmia.

MCG is a non-contact measurement technique whereby the magnetic field data from the heart is recorded at several locations in space—typically above the chest, in a non-contact manner. During device setup, an operator would position an MCG device (e.g., MCG sensors) at a distance (e.g., from 0.5 cm to 5 cm) from a patient. In some instances, during device setup, the operator may record the position of the MCG device relative to the patient by manually measuring the distance between the device and the patient (e.g., using a ruler or a tape measure), or by taking photographs. However, during device operation, the position of the patient relative to the MCG device is not determined or tracked. Furthermore, the MCG data of the patient does not typically contain information about the patient position, except for technician notes or photographs taken prior to the MCG measurements.

From a practical perspective, the relative position between the patient and the MCG device can change during device operation due to movement of the patient, breathing, and at a finer level, during each heartbeat of the patient due to compression of the chest. These movements can affect the quality of MCG recordings and the ability to accurately perform source reconstruction from the collected data.

Despite its strengths over ECG, MCG data tends to have lower signal-to-noise compared to ECG data due to interferences from the environment. This issue is further exacerbated when the MCG device is operating in a magnetically unshielded environment. On the other hand, current ECG protocols lack a suitable method to localize ECG electrode locations on the patient's body during ECG measurements, are heavily reliant on accurate placement of the electrodes by the technician, and assume that the electrodes on the patient are correctly placed by the technician. Even when correctly placed, the electrodes may move or slip during the ECG measurements. Because the quality of the ECG data is dependent on accurate electrode placement, inaccurate estimation of ECG electrode locations can result in negative consequences such as false diagnoses and/or further unnecessary cardiovascular testing.

Accordingly, there is a need for improved systems, devices, and methods that enable a relative position between a MCG device and a patient subject to the MCG scan to be determined (and tracked) while performing the MCG measurements. In particular, the ability to co-register the position of the patient of the MCG measurements with the biomagnetic signals measurements can provide critical information for improving the quality of the MCG data.

There is also a need for improved systems, devices, and methods that can leverage the strengths of both ECG and MCG while addressing the deficiencies of these systems.

Some embodiments of the present disclosure describe an improved system, device and method for magnetometry imaging that facilitates continuous monitoring of patient position during MCG measurements. In some embodiments, the disclosed system and/or device are specifically designed to be operating at room temperature and in an electromagnetically unshielded environment.

As disclosed herein, in some embodiments, one or more electromagnetic transmitters are positioned on a predefined anatomical position (e.g., a landmark position, such as the suprasternal notch, or a numbered intercostal space) of a patient during a biomagnetic field scan of the patient using a MCG system. In some embodiments, the one or more electromagnetic transmitters comprise one or more conductive coils. In some embodiments, the one or more electromagnetic transmitters are coupled to a coil driving system that is capable of driving current through each of the electromagnetic transmitters at a respective, predefined frequency and/or other characteristics. During the biomagnetic field scan of the patient, the MCG system measures the biomagnetic field signals from the patient and the magnetic field(s) generated from the one or more electromagnetic transmitters. The relative amplitude of the signal at each magnetometer position depends on the relative distance between a respective electromagnetic transmitter and the magnetometer. As a respective electromagnetic transmitter moves, either due to patient movement with respect to the MCG sensor array or movement of the patient's chest wall during a heartbeat or breathing, the magnetic field profile of the respective electromagnetic transmitter that is detected by the MCG sensor array changes. In some instances, the amplitude of the magnetic field signals from a respective electromagnetic transmitter that is recorded by the magnetometers may also change. This amplitude modulation is directly related to the distance and angle between the respective electromagnetic transmitter and a respective magnetometer.

As disclosed herein, in some embodiments, the magnetic field signals emitted from a respective electromagnetic transmitter can provide seismocardiogram (e.g., chest wall motion) measurements. In some embodiments, the magnetic field signals emitted from a respective electromagnetic transmitter can be used to determine ballistocardiogram measurements. The seismocardiogram and the ballistocardiogram are independent artifacts in the MCG data that are mixed in with the electromagnetic heartbeat signals. These artifact signals can each be extracted from the electromagnetic transmitter invention signal, so these can be used regress those artifacts from the electromagnetic heartbeat signal in MCG.

Some embodiments of the present disclosure describe a system that has both a MCG modality and an ECG modality, and is capable of concurrently recording electrical activity of the heart using both the modalities. In some embodiments disclosed herein, a hybrid, integrated system includes a MCG subsystem (e.g., MCG system) and an ECG subsystem (e.g., ECG system). The disclosed hybrid system addresses the limitations of current ECG systems because the problem of unknown ECG electrode locations can be inferred by the MCG subsystem using an appropriate magnetic tracking scheme. Furthermore, by using MCG methods to localize the cardiac source location, the ECG electrode locations can be determined relative to the electrophysical activity of the heart, thereby providing key anatomical information about the position and orientation of the heart within the chest cavity, relative to the electrodes, enabling better interpretation of leads. Moreover, the ECG electrode positions can serve as landmarks to estimate the patient's body shape, enabling improved inference of the conductivity of the intermediate tissue between the heart and electrodes. The hybrid system also addresses the limitations of current MCG systems because the low signal-to-noise ratio of the MCG signals can be addressed via averaging of multiple heartbeats together, which is most precisely done with clear timing indications for each R-wave obtained from the ECG data. Moreover, since MCG is a non-contact technique, artifacts from patient movements do not typically have proxies to aid in regression and removal. By utilizing the time resolved ECG locations as regressors, MCG data can be more effectively denoised.

As disclosed herein, in some embodiments, the hybrid system tracks the ECG electrode positions using electromagnetic emission from the electrodes. In some embodiments, the disclosed tracking technique enables continuous localization of the electrodes at a rate that matches movement artifacts from voluntary or involuntary subject motion. The disclosed tracking technique also provides relative continuous positioning information of the ECG electrodes and the MCG sensor array.

As disclosed herein, in some embodiments, the electromagnetic emission tracking approach comprises affixing electromagnetic transmitters to the ECG electrodes (also known as “electrode tracking beacons”) (e.g., by placing current-carrying coils on the ECG electrodes). The MCG subsystem measures magnetic signals emitted by the electrode tracking beacons. Because the relative amplitude of the signal at each magnetometer position depends on the relative distance between beacon and magnetometer, the MCG system may thus accurately infer the offset distance—and dynamically changing distance—between the ECG pads and its own coordinate system.

As disclosed herein, as the ECG electrodes move, whether due to patient movement, or ECG pad sliding, or movement of the patient's chest wall during a heartbeat or breathing, the motion is reflected in the magnetic field data from the electrode tracking beacons, which is recorded alongside the biomagnetic field signals. As a result, the disclosed hybrid system can track each ECG electrode via its respective beacon's signal, finding its location and orientation at each point in time. In some embodiments, in the case of electrodynamic beacons, the hybrid system can solve the magnetic inverse problem. Source localization of the cardiac activity can be inferred by a model that uses both (i) MCG data with its MCG array geometry and (ii) ECG data with ECG electrodes whose locations are co-registered to the patient and to the MCG array geometry.

The systems, methods, and devices of this disclosure each have several innovative aspects, no single one of which is solely responsible for the desirable attributes disclosed herein.

In accordance with some embodiments, a method for determining positional information of a target organ of a human subject during a biomagnetic field scan is performed at a computing device coupled to a plurality of magnetometers, one or more processors, and memory. The method includes detecting, by the plurality of magnetometers, (i) first biomagnetic field signals from at least a portion of the human subject's organ and (ii) second magnetic field signals from an electromagnetic transmitter positioned on a predetermined position of the human subject, where the plurality of magnetometers has a known position relative to each other during the biomagnetic field scan (e.g., each magnetometer has a known positive relative to other magnetometers). The method includes determining a time-varying spatial relationship between the electromagnetic transmitter and the plurality of magnetometers based on the detected second magnetic field signals. The method includes correcting artifacts in the detected first biomagnetic signals in accordance with the determined spatial relationship.

In accordance with some embodiments, a method for determining positional information of a target organ of a human subject during a biomagnetic field scan is performed at a computing device coupled to a plurality of magnetometers, one or more processors, and memory. The method includes detecting, by the plurality of magnetometers: (i) first biomagnetic field signals from at least a portion of the human subject's organ and (ii) second magnetic field signals from an electromagnetic transmitter positioned at a predetermined position of a patient bed of the human subject, where the human subject is physically connected to the patient bed during the biomagnetic field scan and the plurality of magnetometers have a known position relative to each other during the biomagnetic field scan. The method includes determining a time-varying spatial relationship between the electromagnetic transmitter and the plurality of magnetometers based on the detected second magnetic field signals. The method includes correcting artifacts in the detected first biomagnetic signals in accordance with the determined spatial relationship. The motion of the bed, as measured by the transmitter/magnetometer system, will be related to the motion of the patient due to the physical contact between patient and bed. There may be mechanical damping, or latency due to the various materials involved, but the underlying source of the signal (patient motion) is the same.

In accordance with some embodiments, a method for analyzing MCG and ECG data is performed at a system that includes one or more processors, memory, and a magnetocardiography (MCG) subsystem having a plurality of magnetometers. The method includes measuring, via the MCG subsystem using the plurality of magnetometers, MCG data comprising biomagnetic field signals from a human subject. The method includes receiving, from one or more ECG electrodes that are physically coupled to the human subject, ECG data comprising cardiac electrical activity of a heart of the human subject. the method further includes determining location information of the one or more electrodes that are mounted on the human subject.

In accordance with some embodiments, a method is performed at a system that includes a magnetocardiography (MCG) subsystem having a plurality of magnetometers, an electrocardiography (ECG) subsystem having a plurality of electrodes that are physically coupled to a human subject, and an electrode position detector. The method includes measuring, via the plurality of electrodes of the ECG subsystem, ECG data associated with electrical activity of the heart of the human subject. The method includes measuring, via the plurality of magnetometers of the MCG subsystem, biomagnetic field signals from the human subject. The method includes determining, via the electrode position detector, location information of the plurality of electrodes.

In accordance with some embodiments, a system comprises a magnetocardiography (MCG) subsystem that includes a plurality of magnetometers. The system further comprises ne or more processors and memory. The memory stores one or more programs that are for execution by the one or more processors. The system measures, via the MCG subsystem using the plurality of magnetometers, MCG data comprising biomagnetic field signals from a living subject. The system receives, from one or more ECG electrodes that are physically coupled to the living subject, ECG data comprising cardiac electrical activity of the living subject. The system determines location information of the one or more electrodes that are mounted on the living subject. The system enhances the measured MCG data and/or the received ECG data according to determined location information of the one or more electrodes.

In accordance with some embodiments, a system comprises a magnetocardiography (MCG) subsystem including a plurality of magnetometers, an electrocardiography (ECG) subsystem including one or more electrodes, and an electrode position detector. During use of the system, the MCG subsystem is configured to measure MCG data comprising biomagnetic field signals from the living subject via the plurality of magnetometers. The electrode position detector is configured to determine location information of the one or more electrodes that are mounted on the human subject. The ECG subsystem is configured to measure ECG data comprising cardiac electrical activity of the living subject via the one or more electrodes. The system is configured to enhance the measured MCG data and/or ECG data in accordance with the determined location information of the one or more electrodes.

In accordance with some embodiments, a system comprises one or more electrodes configured to be mounted on a human subject. Each electrode is physically coupled to a respective electromagnetic transmitter. When the one or more electrodes are mounted on the human subject, the system is configured to (i) measure, via a plurality of magnetometers that is communicatively coupled to the system, first magnetic field signals from the one or more electromagnetic transmitters; (ii) determine a spatial relationship between the plurality of magnetometers and the one or more electromagnetic transmitters based on the measured first magnetic signals; and (iii) localize respective positions of the one or more electrodes.

In accordance with some embodiments, a system comprises a magnetocardiography (MCG) subsystem having a plurality of magnetometers, one or more processors, and memory. The memory stores one or more programs that are configured for execution by the one or more processors. The one or more programs include instructions for performing any of the methods disclosed herein.

In accordance with some embodiments, a system comprises a magnetocardiography (MCG) subsystem having a plurality of magnetometers, an electrocardiography (ECG) subsystem having a plurality of electrodes that are physically coupled to a human subject, an electrode position detector, one or more processors, memory, and one or more programs stored in the memory for execution by the one or more processors. The one or more programs include instructions for performing any of the methods described herein.

In accordance with some embodiments, a computer system includes one or more processors and memory. In some embodiments, the computer system is coupled to a plurality of magnetometers. In some embodiments, the plurality of magnetometers are part of a MCG system (e.g., a MCG subsystem). In some embodiments, the system is coupled to an ECG system (e.g., an ECG subsystem) having a plurality of ECG electrodes. In some embodiments, the computer system is coupled to an electrode position detector. The memory stores one or more programs configured for execution by the computer system. The one or more programs include instructions for performing any of the methods described herein.

In accordance with some embodiments, a non-transitory computer-readable storage medium stores one or more programs configured for execution by a computer system having one or more processors and memory. The one or more programs include instructions for performing any of the methods described herein.

In accordance with some embodiments, the disclosed systems, devices, and methods have several advantages compared to existing MCG or MEG systems, including and not limited to: (i) operability at room temperature, in a magnetically unshielded environment, (ii) reduced operating costs because there is no need to design a magnetically-shielded environment around the system and/or operate at cryogenic temperatures; (ii) increased usability, including deployment to less-restricted environments with high MCG utility, where patients must be scanned efficiently in ordinary exam rooms and on ordinary beds; (iii) utilization of magnetometers that allow further opportunities for device miniaturization and/or portability.

In accordance with some embodiments, the disclosed systems, devices, and methods are advantageous over optical tracking approaches for identifying the ECG electrode locations on a patient's body. Although optical imaging (e.g., in the visible or IR wavelengths) with cameras, combined with advances in object recognition (e.g., using machine learning), can facilitate full surface mapping of the human body, these methods require the cameras to have a direct line of sight to the ECG electrodes and/or to body landmarks, which may require removal of a patient's clothing or complicated measurement procedures. Furthermore, these one-time measurements do not provide continuous tracking of electrode positions during the course of a measurement. Furthermore, continuous recording of a patient during a medical examination may create patient privacy issues.

In accordance with some embodiments, the disclosed hybrid ECG-MCG system enables source localization of the cardiac activity to be inferred by a model that uses both (i) MCG data with its MCG array geometry and (ii) ECG data (with tracking information of the ECG electrodes) that is co-registered to the patient and to the MCG array geometry. In some embodiments, the model applies joint denoising techniques to obtain clinically relevant insights from the ECG and MCG data.

Note that the various embodiments described above can be combined with any other embodiments described herein. The features and advantages described in the specification are not all inclusive and, in particular, many additional features and advantages will be apparent to one of ordinary skill in the art in view of the drawings, specification, and claims. Moreover, it should be noted that the language used in the specification has been principally selected for readability and instructional purposes and may not have been selected to delineate or circumscribe the inventive subject matter.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1A illustrates an operating environment in accordance with some embodiments.

FIG. 1B illustrates an apparatus for measuring magnetic fields from a subject's organ in accordance with some embodiments.

FIG. 2 illustrates a block diagram of a device for measuring magnetic fields of a target organ of a human subject according to some embodiments.

FIG. 3 is a block diagram illustrating a computer system, in accordance with some embodiments.

FIG. 4 illustrates an exemplary setup that enables a relative position between magnetometers of a MCG system and a human subject of the MCG measurements to be continuously determined during the MCG measurements, in accordance with some embodiments.

FIG. 5A illustrates a magnetic field distribution due to current flowing through a loop of coils in an XY plane, in accordance with some embodiments.

FIG. 5B illustrates the dimensionless modulation depth in a case where a current-carrying coil is displaced at a distance r from a magnetometer along the coil axis, in accordance with some embodiments.

FIG. 6 illustrates a graph showing actual raw time-series magnetic field signals collected from an array of magnetometers by placing the array over a human subject at room temperature and in a magnetically unshielded environment, in accordance with some embodiments.

FIG. 7A is a graph showing raw magnetic field signals detected by a multi-channel magnetometer array over a one-second duration, by placing the magnetometer array above the chest of a human test participant whose chest is affixed with a CPI coil, in accordance with some embodiments.

FIG. 7B is a graph showing respective average MCG signals extracted from the raw magnetic field signals in FIG. 7A, for all magnetometer channels, in accordance with some embodiments.

FIG. 7C shows a power spectral density of the raw magnetometer signals in accordance with some embodiments.

FIG. 8A illustrates a graph that includes a line plot corresponding to the output of phase sensitive demodulation of magnetometer signals (blue), acquired from a magnetometer placed above a chest of a human test participant whose chest is affixed with a CPI coil, in accordance with some embodiments. The graph also includes a line plot corresponding to the synchronously acquired ECG signal from the same participant (orange).

FIG. 8B illustrates a graph that includes a line plot corresponding to filtered, demodulated magnetic field signals, in accordance with some embodiments.

FIG. 8C illustrates a graph that includes a line plot corresponding to a longer segment of the filtered, demodulated magnetic field data that is shown in FIG. 8B, in accordance with some embodiments.

FIG. 9A is a graph showing a filtered magnetic field signal generated by a CPI coil that is affixed to a human subject, after application of a 2 Hz high-pass filter to the raw CPI magnetic field signals.

FIG. 9B is a graph showing a filtered magnetic field signal that is generated by applying a 5 Hz high-pass filter to the same raw CPI magnetic field signals for FIG. 8A.

FIG. 10A is a graph showing variations in an amplitude of a magnetic field signal generated by CPI coil over time, in accordance with some embodiments.

FIG. 10B is a graph of the same magnetic field data in FIG. 10A after filtering to remove low-frequency content.

FIG. 11 illustrates a power spectral density plot of the demodulated CPI coil signal of FIG. 8A, in accordance with some embodiments.

FIG. 12 is a schematic diagram of a hybrid system (e.g., a combined system) for performing MCG and ECG measurements, in accordance with some embodiments.

FIGS. 13A and 13B collectively illustrate a data flow process for a hybrid system that includes an ECG system and a MCG system, in accordance with some embodiments.

FIG. 14 is an image illustrating standard placement locations for ECG electrodes, in accordance with some embodiments.

FIGS. 15A to 15D illustrate a flowchart diagram for a method of determining positional information of a target organ of a human subject during a biomagnetic field scan, in accordance with some embodiments.

FIG. 16 illustrates a flowchart diagram for a method for determining positional information of a target organ of a human subject during a biomagnetic field scan, in accordance with some embodiments.

FIGS. 17A to 17D illustrate a flowchart diagram for a method of performing continuous, colocalized electrophysiology measurements on a human subject, in accordance with some embodiments.

FIGS. 18A to 18D illustrate a flowchart diagram for a method of performing continuous, colocalized electrophysiology measurements on a human subject, in accordance with some embodiments.

Reference will now be made to embodiments, examples of which are illustrated in the accompanying drawings. In the following description, numerous specific details are set forth in order to provide a thorough understanding of the present invention. However, it will be apparent to one of ordinary skill in the art that the present invention may be practiced without requiring these specific details.

### DETAILED DESCRIPTION

Some methods, devices, and systems disclosed in the present specification advantageously improve upon existing devices for bio-imaging, by providing a device and/or system that combines hardware components and signal processing techniques for resolving magnetic fields from a target organ of a human subject.

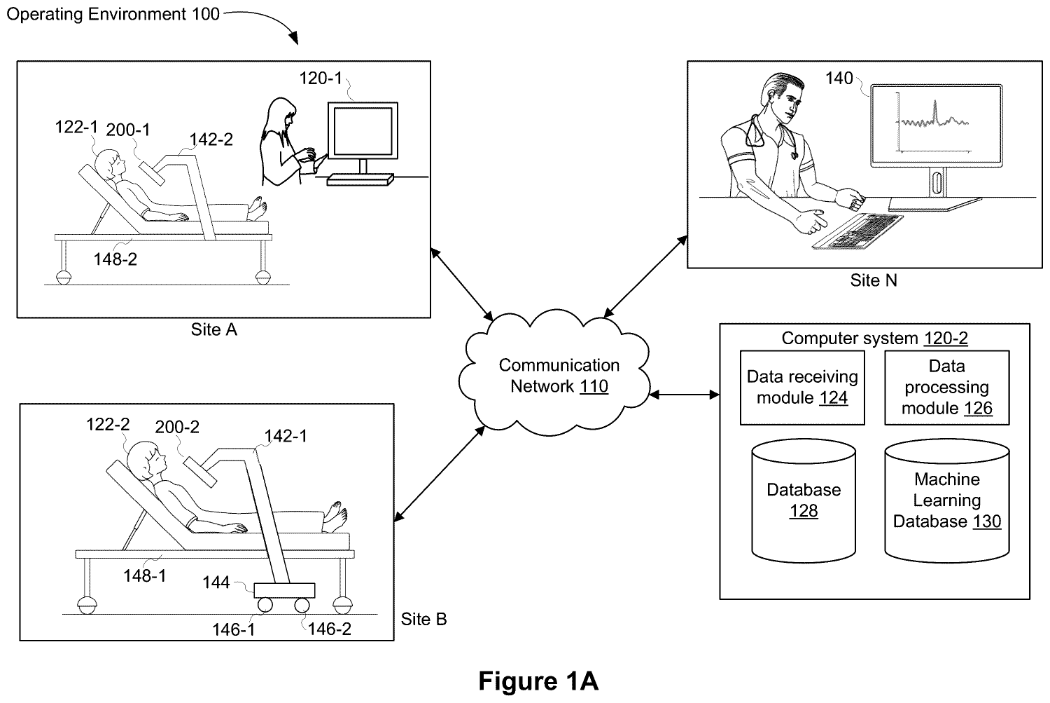

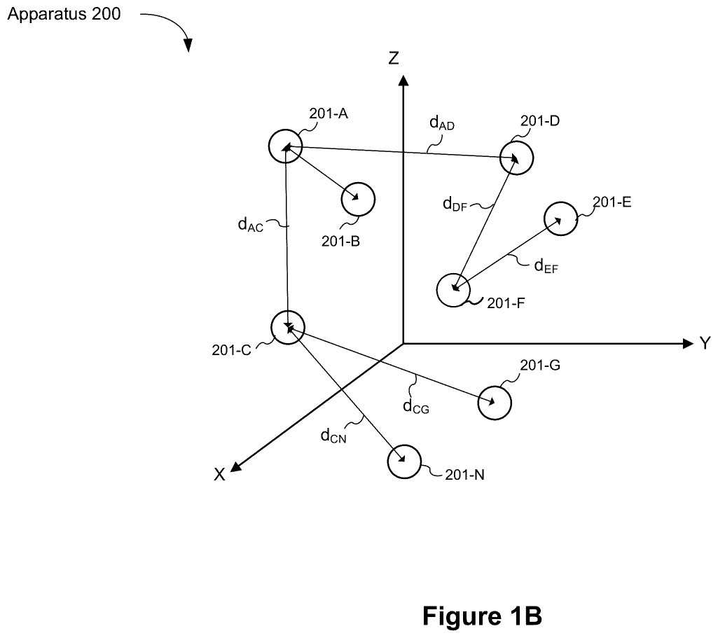

FIG. 1A illustrates an exemplary operating environment 100 in accordance with some embodiments. In some embodiments, the operating environment 100 includes one or more devices 200. In some embodiments, the device 200 is a MCG device (or an MCG system) for measuring magnetic signals from the heart of a human subject. In the example of FIG. 1A, each of the devices 200 (e.g., device 200-1 and device 200-2) is located at a respective (e.g., distinct) site (e.g., Site A and Site B), corresponding to a respective (e.g., distinct) city, state, or country. In some embodiments, a site corresponds to a hospital, a clinic, or a medical facility. In some embodiments, a respective site comprises a room-temperature environment. In some embodiments, a respective site is electromagnetically-unshielded.

In some embodiments, the device 200 (e.g., an apparatus) is communicatively coupled through communication network(s) 110 to a computer system 120. In some embodiments, the computer system 120 and the device 200 are co-located at the same location (e.g., in the same room where the device 200 is employed). For example, FIG. 1A shows that the device 200-1 and the computer system 120-1 are located in Site A (e.g., different rooms of the same hospital, or within the same room in a hospital). In some embodiments, the device 200 and the computer system 120 are located at different locations. For example, FIG. 1A shows that the device 200-2 is located at site B whereas the computer device 120-2 is located at site N. In some embodiments, the computer system is a system that is located on the cloud.

As disclosed herein, the device 200 includes one or more magnetometers 201 that are configured to measure (e.g., sense, acquire, collect, obtain, detect etc.) magnetic field signals from a target organ of a human subject 122 while the device 200 is operating in a magnetically unshielded environment. In addition to measuring the magnetic field signals from the human subject, the one or more magnetometers 201 are also configured to measure magnetic field signals from other magnetic field-emitting sources in the vicinity of the magnetometers. In some embodiments, the measured signals are transmitted to the computing system 120 for post-processing. In some embodiments, the measured signals are processed locally on the device 200. In some embodiments, the data (e.g., actual data or post-processed data) is caused to be displayed on the computer system 120, or on a display device 140 that is communicatively connected to the computer system 120.

In some embodiments, the device 200 is a part of a self-contained device/system that also includes sensors (e.g., magnetometers 201, and optionally, sensors 203 of different sensor types, such as light sensors, motion sensors, accelerometers, etc.), one or more positioning arms (e.g., mechanical positioning arm) (e.g., positioning arm 142-1 or positioning arm 142-2, FIG. 1A), a computer (e.g., computer system 120, display device 140, etc.), and power supply with backup.

In some embodiments, the self-contained device/system 200 is a mobile device/system that is configured to be movable within the operating environment 100. For example, FIG. 1A illustrates that, in some embodiments, the device/system 200 includes a standalone positioning arm 142-1 mounted on a base 144 that includes one or more wheels 146 (e.g., wheel 146-1 and 146-2). The device/system 200 can be moved between different examination rooms within the operating environment 100, where it can be positioned next to a patient lying on a patient support platform 148-1 or next to a patient sitting on a chair. FIG. 1A also illustrates that, in some embodiments, the device/system 200 includes a positioning arm 142-2 that can be directly mounted on a patient support platform 148-2. The device/system 200 is designed such that it can acquire data (e.g., imaging data, scan data, etc.) from the patient without requiring the patient to relocate from their present position, thereby minimizing disruptions to the critical care workflow.

In some embodiments, the apparatus 200 includes a panel (e.g., rigid panel) on (or within) which the magnetometers 201 are mounted. During device operation, the panel maintains a fixed position relative to the patient. For example, when the apparatus 200 is a MCG device, the apparatus 200 (e.g., the panel including the magnetometers 201) can maintain a position with a 0.5 cm to 5 cm parallel gap to the patient's chest during an entire patient scan, with minimal vibration (e.g., no vibration) and/or minimal slippage (e.g., no slippage, slippage/movement no greater than 1 mm, etc.) during the scan. In some embodiments, the device stability is assisted by locking out unnecessary mechanical degrees of freedom (DOF) (e.g., no rotation around the y-axis and/or z-axis).

Further elaborating on the implementation in which the apparatus 200 is a MCG device, in some embodiments, with the base of the apparatus 200 stabilized with respect to the floor and parallel to a patient's sagittal plane, an operator can easily and conveniently (e.g., within seconds of manual or automatic actuation) maneuver the positioning arm 142 (e.g., positioning arm 142-1) to bring the sensor panel substantially close to, and parallel with, the patient's chest. In some embodiments, the apparatus 200 enables easy and accurate positioning of the panel such that it is positioned substantially centered over a heart of the patient. For example, in some embodiments, the apparatus 200 enables fiducial registration to standard anatomical landmarks to enable data consistency between patients.

In some embodiments, the positioning arm 142 (e.g., positioning arm 142-1 or 142-2) includes lockable moving degrees of freedom (DOF) (e.g., three coupled DOF, namely: sliding along the y-axis, and a double-jointed pivot allowing coarse rotation around the x-axis followed by fine rotation around the x-axis).

In some embodiments, the device/system 200 is configured to acquire a biomagnetic field scan of a patient and provide information about potential disease states. In some embodiments, the patient scan and diagnosis are provided during the same patient visit. For example, in some embodiments, the device/system 200 is configured to acquire a biomagnetic field scan of a patient within one, two, or three minutes of scan initiation. In some embodiments, the device/system 200 is configured to process the scan data locally (e.g., in real time), or transmit the scan data to the cloud for processing (e.g., in real time). In some embodiments, care-informing results are returned (e.g., by the computer system 120) to the operator within two to five minutes after that. In some embodiments, electrocardiography (ECG) data of the patient is acquired during the patient visit. In some embodiments, the ECG data of the patient is acquired concurrently with the biomagnetic field scan.

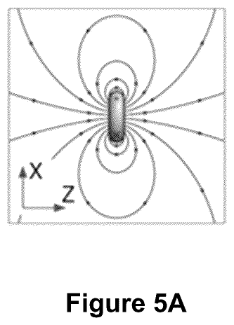

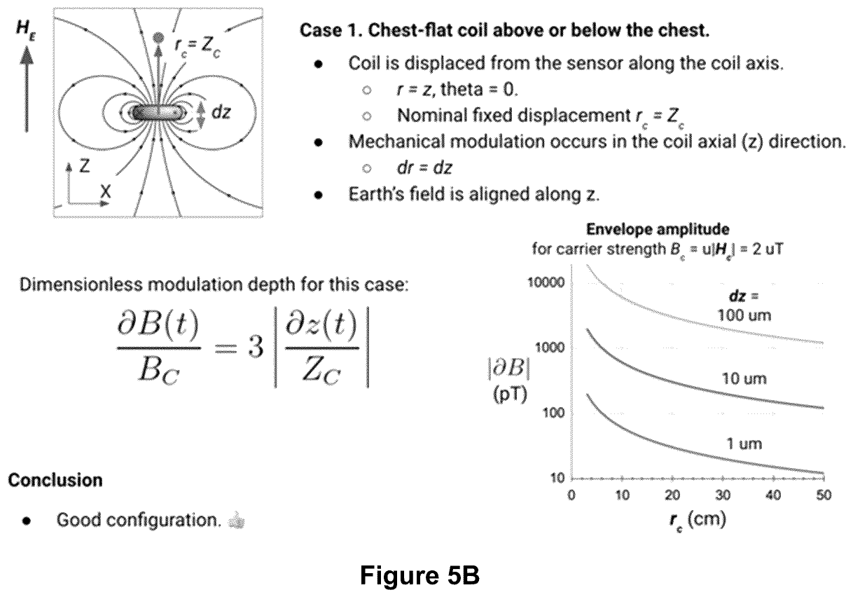

In the example of FIG. 1A, the computer system 120 includes a data receiving module 124 that is configured to receive data from one or more devices 200. In some embodiments, the data corresponds to magnetic field data that is generated (e.g., measured, collected, acquired) from respective magnetometers 201 of a device 200. The computer system 120 includes a data processing module 126 that is configured to process the received data. Further details of the data processing process are described with respect to FIGS. 5 to 11.

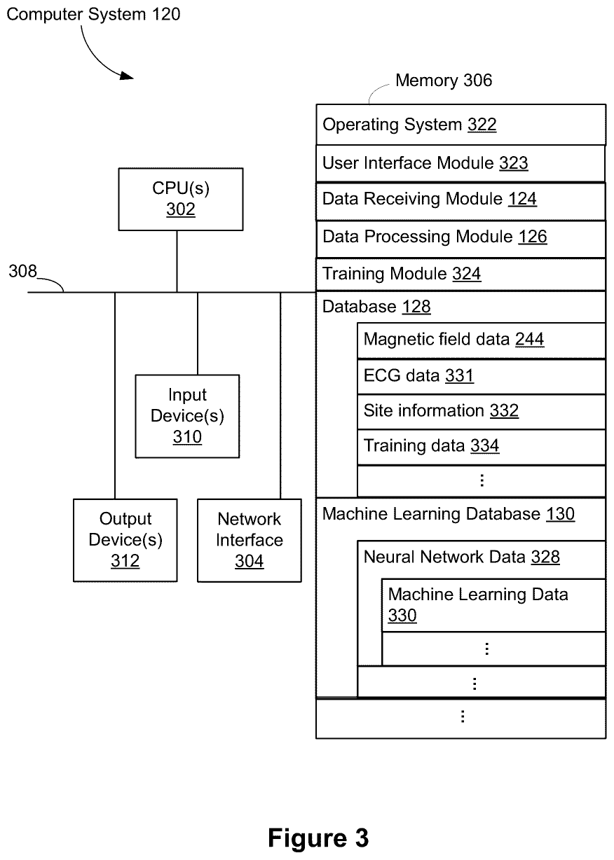

In some embodiments, the computer system 120 includes a database 128. Details of the database 128 are described with respect to FIG. 3.

In some embodiments, the computer system 120 includes a machine learning database 130 that stores machine learning information. In some embodiments, the machine learning database 130 is a distributed database. In some embodiments, the machine learning database 130 includes a deep neural network database. In some embodiments, the machine learning database 130 includes supervised training and/or reinforcement training databases.

FIG. 1B illustrates an apparatus 200 for measuring magnetic fields from a subject's organ in accordance with some embodiments. In this example, the apparatus 200 occupies a three-dimensional space that is defined by three orthogonal axes (x-, y-, and z-axes). The apparatus 200 includes a plurality of magnetometers 201 (e.g., magnetometer 201-A to magnetometer 201-N). Each of the magnetometers 201 occupies a respective position in the three-dimensional space of the apparatus 200, with respective x-, y-, and z-coordinates. A respective pair of magnetometers 201-i and 201-j, in the plurality of magnetometers 201, is separated by a respective distance dij. For example, FIG. 1B shows that the pair of magnetometers 201-E and 201-F is separated by a distance d_{EF}, and the pair of magnetometers 201-C and 201-N is separated by a distance d_{CN}. Each magnetometer in the plurality of magnetometers 201 is configured to simultaneously detect a biomagnetic field from at least a portion of the subject's organ and a background magnetic field.

In some embodiments, each of the magnetometers has a fixed (e.g., rigid) position in the apparatus 200. A respective pair of magnetometers 201-i and 201-j, in the plurality of magnetometers 201, is separated by a respective fixed distance dij.

In some embodiments, the apparatus 200 can include at least one magnetometer 201 whose position is variable (e.g., continuously variable). The positions of the magnetometers can be tracked during device operation.

In some embodiments, each of the magnetometers 201 in the apparatus 200 is responsive to a total magnetic field in proximity to the magnetometer 201. During device operation, the magnetometers 201 detect biomagnetic fields from a subject's organ as well as background magnetic field (e.g., from the earth and/or other interference sources). In some embodiments, the magnetometer 201 is a scalar magnetometer that measures the total strength of the magnetic field to which it is subjected, but not the direction. In some embodiments, the magnetometer 201 is a vector magnetometer that is capable of measuring the components of the magnetic field in a particular direction, relative to the spatial orientation of the magnetometer.

In some embodiments, the magnetometers 201 in the apparatus have an average spacing that satisfies a constraint in Fourier space. For example, in some embodiments, the average magnetometer spacing is determined by the Nyquist sampling rate in Fourier space of the wavevectors of the target organ's magnetic field. In some embodiments, the magnetometers 201 are spatially distributed in (e.g., within) the apparatus 200 such that in Fourier space, the magnetometers 201 have a wavevector coverage to recover information from both the biomagnetic field from the subject's organ and the background magnetic field.

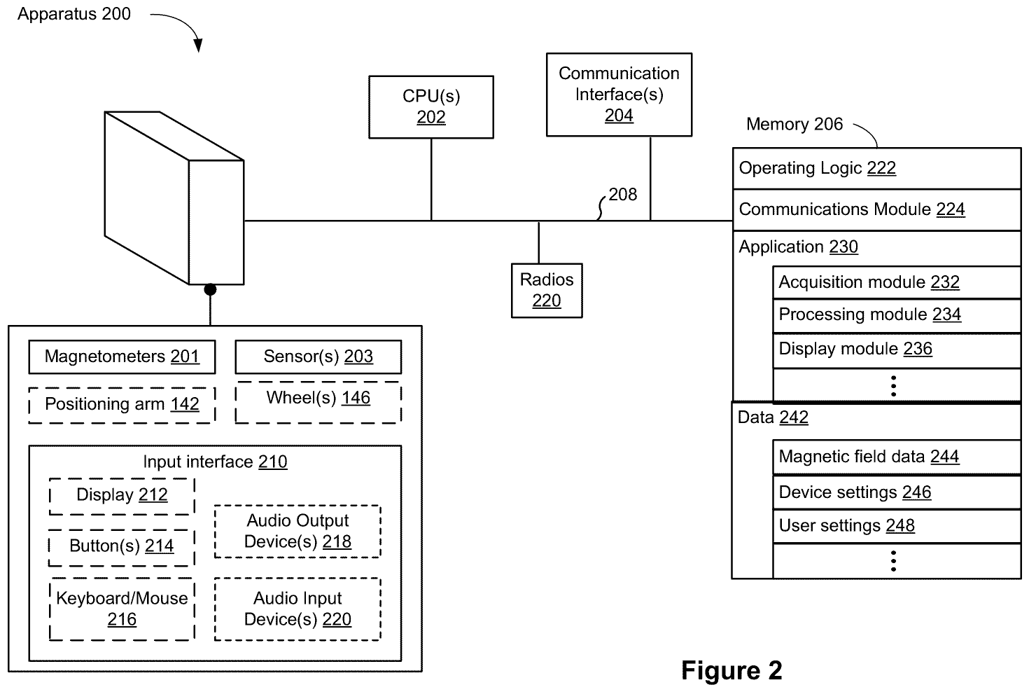

FIG. 2 shows a block diagram of a device 200 (e.g., an apparatus) for measuring magnetic fields of a target organ of a human subject according to some embodiments. In some embodiments, the device 200 is a MCG device (or MCG system). In some embodiments, device 200 is part of a hybrid system that includes MCG and electrocardiography (ECG) capabilities.

The device 200 includes one or more magnetometers 201 (e.g., a magnetic field sensor).

In some embodiments, the magnetometer 201 comprises an electron spin defect based magnetometer, such as a diamond nitrogen vacancy (NV) center magnetometer (e.g., a solid state sensor). A diamond NV center magnetometer is a quantum sensor that leverages the occurrence of an electronic spin defect in a solid state lattice, where the spin can be both initialized and read out optically or electronically. In some instances, the defect may arise as an atomic-level vacancy in a lattice structure, such as a vacancy occurring near a nitrogen atom that is substituted in place of a carbon atom within diamond.

In some embodiments, the magnetometer 201 comprises an optically pumped magnetometer (OPM) (e.g., a vapor cell sensor). An OPM is a quantum sensor that includes a heated alkali vapor (including, and not limited to, a caesium vapor, a rubidium vapor, or a potassium vapor), through which a laser beam passes through. Due to the quantum properties of the atoms, the amount of light passing through the atomic vapor is modulated at a frequency that is proportional to the environmental magnetic field.

In some embodiments, the magnetometer 201 comprises a fluxgate sensor.

In some embodiments, the magnetometer 201 is responsive to a total magnetic field of the magnetometer 201 (e.g., at the atomic vapor). During device operation, the magnetometer detects a total magnetic field, including biomagnetic fields from a subject's organ as well as background magnetic field (e.g., from the earth, other equipment in the vicinity of the total magnetometer etc.). In some embodiments, the magnetometer 201 is a scalar magnetometer that measures the total strength of the magnetic field to which it is subjected, but not the direction. In some embodiments, the magnetometer 201 is a vector magnetometer that is capable of measuring both the magnitude of the magnetic field as well as the respective field direction(s). In some instances, the total strength of the magnetic field can be obtained by calibrating and processing the plurality of signals according to physical sensor orientation (e.g., computing a dot product of the vector components).

In some embodiments, the one or more magnetometers 201 comprise at least two magnetometers that are arranged in an array. In some embodiments, the array of magnetometers is arranged as a stack of planes.

In some embodiments, the device 200 includes one or more sensors 203 that are distinct from the magnetometers 201. For example, the one or more sensors 203 can include light sensors, motion sensors and/or accelerometers.

In some embodiments, the device 200 includes a positioning arm 142, as described with respect to FIG. 1A. In some embodiments, the device 200 includes one or more wheels 146 for supporting the device 200 (and the positioning arm 142), as described with respect to FIG. 1A.

In some embodiments, the device 200 includes an input interface 210 for facilitating user input, such as a display 212, button(s) 214, a keyboard and/or mouse 216.

In some embodiments, input interface 210 includes a display device 212. In some embodiments, the device 200 includes input devices such as button(s) 214, and/or a keyboard/mouse 216. Alternatively or in addition, in some embodiments, the display device 212 includes a touch-sensitive surface, in which case the display device 212 is a touch-sensitive display. In some embodiments, the touch-sensitive surface is configured to detect various swipe gestures (e.g., continuous gestures in vertical and/or horizontal directions) and/or other gestures (e.g., single/double tap). In computing devices that have a touch-sensitive display, a physical keyboard is optional (e.g., a soft keyboard may be displayed when keyboard entry is needed). The input interface 210 also includes an audio output device 218, such as speakers or an audio output connection connected to speakers, earphones, or headphones. In some embodiments, the device 200 uses a microphone and voice recognition to supplement or replace the keyboard/mouse 216. In some embodiments, the apparatus 200 includes audio input device(s) 220 (e.g., a microphone) to capture audio (e.g., speech from a user).

The device 200 also includes one or more processors (e.g., CPU(s)) 202, one or more communication interface(s) 204 (e.g., network interface(s)), memory 206, and one or more communication buses 208 for interconnecting these components (sometimes called a chipset).

In some embodiments, the device 200 includes radios 219. The radios 219 enable one or more communication networks, and allow the device 200 to communicate with other devices, such as a computer system (e.g., the computing system 120 in FIGS. 1 and 3), or a server, an ECG device, and/or other devices 200. In some embodiments, the radios 219 are capable of data communication using any of a variety of custom or standard wireless protocols (e.g., IEEE 802.15.4, Wi-Fi, ZigBee, 6LoWPAN, Thread, Z-Wave, Bluetooth Smart, ISA100.5A, WirelessHART, MiWi, Ultrawide Band (UWB), and/or software defined radio (SDR)) custom or standard wired protocols (e.g., Ethernet or HomePlug), and/or any other suitable communication protocol, including communication protocols not yet developed as of the filing date of this patent application.

The memory 206 includes high-speed random access memory, such as DRAM, SRAM, DDR RAM, or other random access solid state memory devices. In some embodiments, the memory includes non-volatile memory, such as one or more magnetic disk storage devices, one or more optical disk storage devices, one or more flash memory devices, or one or more other non-volatile solid state storage devices. In some embodiments, the memory 206 includes one or more storage devices remotely located from one or more processor(s) 202. The memory 206, or alternatively the non-volatile memory within the memory 206, includes a non-transitory computer-readable storage medium. In some embodiments, the memory 206, or the non-transitory computer-readable storage medium of the memory 206, stores the following programs, modules, and data structures, or a subset or superset thereof:
operating logic 222, including procedures for handling various basic system services and for performing hardware dependent tasks;
a communication module 224 (e.g., a radio communication module), which connects to and communicates with other network devices (e.g., a local network, such as a router that provides Internet connectivity, networked storage devices, network routing devices, server systems, computer system 120, and/or other connected devices) coupled to one or more communication networks via the communication interface(s) 204 (e.g., wired or wireless);
an application 230, which acquires magnetic field data (e.g., via the magnetometers 201) and/or processes the acquired data. In some embodiments, the application 230 controls one or more components of the device 200 and/or other connected devices (e.g., in accordance with the acquired data). In some embodiments, the application 230 includes:
   an acquisition module 232, which acquires magnetic data (e.g., magnetic field data) from magnetometers 201. In some embodiments, the magnetic data comprise time-series magnetic data;
   a processing module 234, which processes the magnetic field data;
   a display module 236, which generates visualizations of the magnetic field data for display by the display 212 and/or the output device(s) 312;
data 242 for the apparatus 200, including but not limited to:
   magnetic field data 244. In some embodiments, the magnetic field data 244 is acquired by the device 200, the MCG system 402, and/or system 1200. In some embodiments, the magnetic field data 244 includes magnetic field spectra and/or magnetic field maps;
   device settings 246 for the device 200, such as default options, acquisition settings, and preferred user settings; and
   user settings 248.

In some embodiments, data collected during the data acquisition process is added as data 242.

Although FIG. 2 shows a device (e.g., an apparatus) 200, FIG. 2 is intended more as a functional description of the various features that may be present rather than as a structural schematic of the embodiments described herein. In practice, and as recognized by those of ordinary skill in the art, items shown separately could be combined and some items could be separated.

Each of the above identified executable modules, applications, or sets of procedures may be stored in one or more of the previously mentioned memory devices, and corresponds to a set of instructions for performing a function described above. The above identified modules or programs (i.e., sets of instructions) need not be implemented as separate software programs, procedures, or modules, and thus various subsets of these modules may be combined or otherwise re-arranged in various embodiments. In some embodiments, the memory 206 stores a subset of the modules and data structures identified above. Furthermore, the memory 206 may store additional modules or data structures not described above. In some embodiments, a subset of the programs, modules, and/or data stored in the memory 206 are stored on and/or executed by a server system, and/or by an external device (e.g., computer system 120).

FIG. 3 is a block diagram illustrating a computer system 120, in accordance with some embodiments.

In some embodiments, the computer system 120 is located at the same location (e.g., in the same site or in the same room) as the device 200 (e.g., computer device 120-1 in FIG. 1A). In some embodiments, the computer system 120 is located at a location that is distinct from a location of the device 200 (e.g., computer device 120-2 in FIG. 1A).

The computer system 120 includes one or more processors 302 (e.g., processing units of CPU(s)), one or more network interfaces 304, memory 306, and one or more communication buses 308 for interconnecting these components (sometimes called a chipset), in accordance with some embodiments.

The computer system 120 optionally includes one or more input devices 310 that facilitate user input, such as a keyboard, a mouse, a voice-command input unit or microphone, a touch screen display, a touch-sensitive input pad, a gesture capturing camera, or other input buttons or controls. In some embodiments, the computer system 120 optionally uses a microphone and voice recognition or a camera and gesture recognition to supplement or replace the keyboard. The computer system 120 optionally includes one or more output devices 312 that enable presentation of user interfaces and display content, such as one or more speakers and/or one or more visual displays.

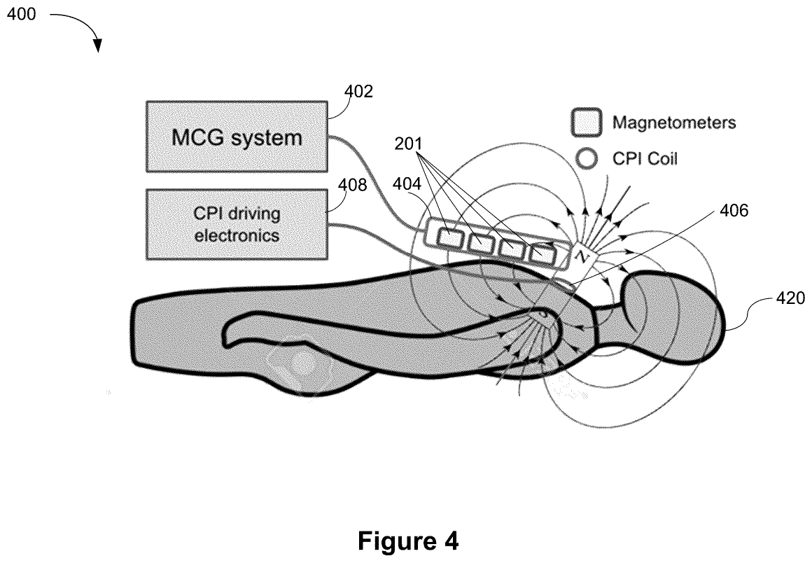

The memory 306 includes high-speed random access memory, such as DRAM, SRAM, DDR RAM, or other random access solid state memory devices; and, optionally, includes non-volatile memory, such as one or more magnetic disk storage devices, one or more optical disk storage devices, one or more flash memory devices, or one or more other non-volatile solid state storage devices. The memory 306, optionally, includes one or more storage devices remotely located from the one or more processors 302. The memory 306, or alternatively the non-volatile memory within the memory 306, includes a non-transitory computer-readable storage medium. In some embodiments, the memory 306, or the non-transitory computer-readable storage medium of the memory 306, stores the following programs, modules, and data structures, or a subset or superset thereof:
an operating system 322 including procedures for handling various basic system services and for performing hardware dependent tasks;
a user interface module 323 for enabling presentation of information (e.g., a graphical user interface for presenting application(s), widgets, websites and web pages thereof, games, audio and/or video content, text, etc.) either at the computer system or at a device, e.g., device 200;
a data receiving module 124 for receiving data (e.g., from device 200, setup 400, or system 1200);
a data processing module 126 for processing data received by the computer system 120. Further details of data processing are described with respect to FIGS. 4, 12, 13A and 13B;
a training module 324 for generating and/or training models, which can be used for identifying and filtering out interference sources to improve the signal-to-noise ratio of magnetic field data, and/or for identifying disease states of the target organ from the magnetic field measurements. In some embodiments, the training module 324 uses training data 334 to generate and/or train the models. In some embodiments, the training data 334 (e.g., training data sets) are generated from measurements from devices 200 (e.g., magnetometers 201). For example, in some embodiments, supervised machine learning is used to aid in denoising and signal and/or feature extraction. In some embodiments, deep learning networks, such as convolutional neural networks (CNNs) or autoencoder networks, are trained on signal components that can be classified as noise or heart-beat signals, and these models can be used to classify signal components in data. Further transformations on training data, which may encompass components derived from principal component analysis (PCA) or independent component analysis (ICA), as well as data augmented with simulated signals or noise, can be used to further refine the models;
a database 128, which store data used, received, and/or created by the computer system 120. In some embodiments, the database includes:
   magnetic field data 244;
   ECG data 331 (e.g., acquired by ECG system 1206 and/or system 1200);
   Site information 332; and
   training data 334.

In some embodiments, the memory 306 includes a machine learning database 130 for storing machine learning information. In some embodiments, the machine learning database 130 includes the following datasets or a subset or superset thereof:
neural network data 328 including information corresponding to the operation of one or more neural network(s). In some embodiments, the neural network data 328 includes one or more models that are trained on a combination of realistic physics simulation of magnetic interference sources and real-world data sets. In some embodiments, the models can identify sources of noise and remove them from the magnetic signals. In some embodiments, the models are trained on a combination of time-series and magnetic field map data to classify different cardiac disease states. In some embodiments, the neural network data 328 includes:
   training data 330, such as training datasets for training the one or more models to identify differences between disease states (e.g., as labeled by trained professionals). In some embodiments, the training data 330 includes signal data (e.g., from target organs) and noise data.

In some embodiments, the computer system 120 includes a device registration module for registering devices (e.g., computer devices, devices 200, etc.) for use with the computer system 120.

Each of the above identified elements may be stored in one or more of the memory devices described herein, and corresponds to a set of instructions for performing the functions described above. The above identified modules or programs need not be implemented as separate software programs, procedures, modules or data structures, and thus various subsets of these modules may be combined or otherwise re-arranged in various embodiments. In some embodiments, the memory 306, optionally, stores a subset of the modules and data structures identified above. Furthermore, the memory 306 optionally stores additional modules and data structures not described above. In some embodiments, a subset of the programs, modules, and/or data stored in the memory 306 are stored on and/or executed by the device 200, the setup 400, or the system 1200.

Electromagnetic Transmitters for Continuous Monitoring of Patient Position during Magnetocardiography Scanning

Some embodiments of the present disclosure are directed to systems, methods, and devices for continuously determining (e.g., tracking) a relative position between magnetometers of a MCG system (e.g., device 200) and a patient subject of the system during MCG measurements.

MCG is a non-contact measurement technique that measures magnetic field signals from a patient subject's heart. In some instances, prior to MCG data acquisition, an operator may record the position of the patient relative to the MCG sensors (e.g., magnetometers). However, the position of the patient relative to the magnetometers, and changes to the position, are typically not monitored during the actual data acquisition. Generally speaking, the relative position between the patient and the magnetometers can change during device operation due to movement of the patient on the patient bed, as well as due to finer movements such as breathing and chest wall compressions. These movements can affect the quality of the MCG recordings and the ability to accurately perform source reconstruction.

According to some embodiments of the present disclosure, a system that includes a plurality of magnetometers can be used to detect first biomagnetic field signals from a patient's heart and detect (e.g., simultaneously, or synchronously with the first biomagnetic field signals) second magnetic field signals from one or more electromagnetic transmitters that are affixed to a predetermined position of the patient during a MCG scan. The system can determine positional information of the patient subject in accordance with the second magnetic field signals. In some embodiments, the system can continuously track the one or more electromagnetic transmitters by determining a respective location and orientation for each of the one or more electromagnetic transmitters at each point in time. In some embodiments, the positional information of the patient subject is co-registered with positional information of the magnetometers. The disclosed system is superior over existing systems because, as will be described later, the ability to co-register the position of the patient of the MCG measurements with the biomagnetic signals measurements can provide critical information for improving the quality of the MCG data.

FIG. 4 illustrates an exemplary setup 400 that enables a relative position between magnetometers 201 of a MCG system 402 (e.g., MCG device 200) and a human subject 420 of the MCG measurements to be continuously determined during the MCG measurements, in accordance with some embodiments. The magnetometers 201 are configured to acquire biomagnetic field signals (e.g., MCG measurements) from the heart of the human subject 420. In some embodiments, the magnetometers 201 are arranged in an array 404 whereby the position of a respective magnetometer relative to other magnetometers in the array 404 is known. An (e.g., at least one) electromagnetic transmitter 406 is positioned (e.g., affixed, using an adhesive such as glue or tape) on a predetermined position of the human subject 420. In some embodiments, the predetermined position is an anatomical landmark position, such as the sternum, or a suprasternal notch (e.g., where the second rib meets the sternum), or the flank, or any other appropriate location that can be used to identify the anatomy of the human subject 420.

In some embodiments, the electromagnetic transmitter 406 comprises a coil (e.g., a conductive coil of wires) having a diameter in the range of about 0.1 cm to 5 cm. The coil can be encased in a biocompatible material such as alumina, a polymer material, or medical-grade silicone. In some embodiments, the electromagnetic transmitter 406, also referred to herein as a chest position indicator (CPI) coil, is coupled to a CPI driving system 408 that is configured to drive an electric current (e.g., an alternating current) through the electromagnetic transmitter 406. The magnetometers 201 are configured to detect signals (e.g., periodic signals) that are emitted by the electromagnetic transmitter 406.

In some embodiments, the CPI driving system 408 is configured to drive an electric current through the electromagnetic transmitter 406 at a predefined frequency (e.g., carrier frequency) that is within a signal collection bandwidth of the magnetometers. For example, an OPM has a signal collection bandwidth that is typically in the range of 1-300 Hz. In some embodiments, signal collection bandwidth of the magnetometers can be in the range of 1-400 Hz, 1-500 Hz, 1-1000 Hz, or 1-2000 Hz.

In some embodiments, the frequency of the current through the electromagnetic transmitter 406 has an upper limit of 5000 Hz, 4000 Hz, 3000 Hz, 2000 Hz, 1000 Hz, 900 Hz, 800 Hz, 700 Hz, 600 Hz, 500 Hz, 400 Hz, 300 Hz, 200 Hz, or 100 Hz.

In some embodiments, the frequency of the current through the electromagnetic transmitter 406 has an upper limit that is up to any frequency within the signal bandwidth of the magnetometers 201 used for the MCG measurements.

While the frequency of the current through the electromagnetic transmitter 406 can fall anywhere within the signal collection bandwidth of the magnetometers 201, it should ideally be outside of the typical spectral band of biomagnetic signals of the heart (e.g., 1-50 Hz) and should avoid common harmonics of electrical line noise (e.g., 50 Hz, 100 Hz, and 150 Hz; or 60 Hz, 120 Hz, and 180 Hz). An example frequency of the current is 170 Hz. In some embodiments, a higher frequency is preferred because it increases the temporal resolution of motion and because the signal-to-noise ratio of the carrier will be higher.

In some embodiments, the electric current through the electromagnetic transmitter 406 has a predefined waveform (e.g., pattern or tone). Exemplary waveforms can include, and are not limited to, a sinusoidal waveform, a square waveform, a triangle waveform, or a sawtooth waveform.

In some embodiments, the setup 400 comprises a plurality of electromagnetic transmitter 406 (e.g., a plurality of coils). Each electromagnetic transmitter of the plurality of electromagnetic transmitters may be uniquely identified based on the properties of a respective electromagnetic transmitter's driving frequency.

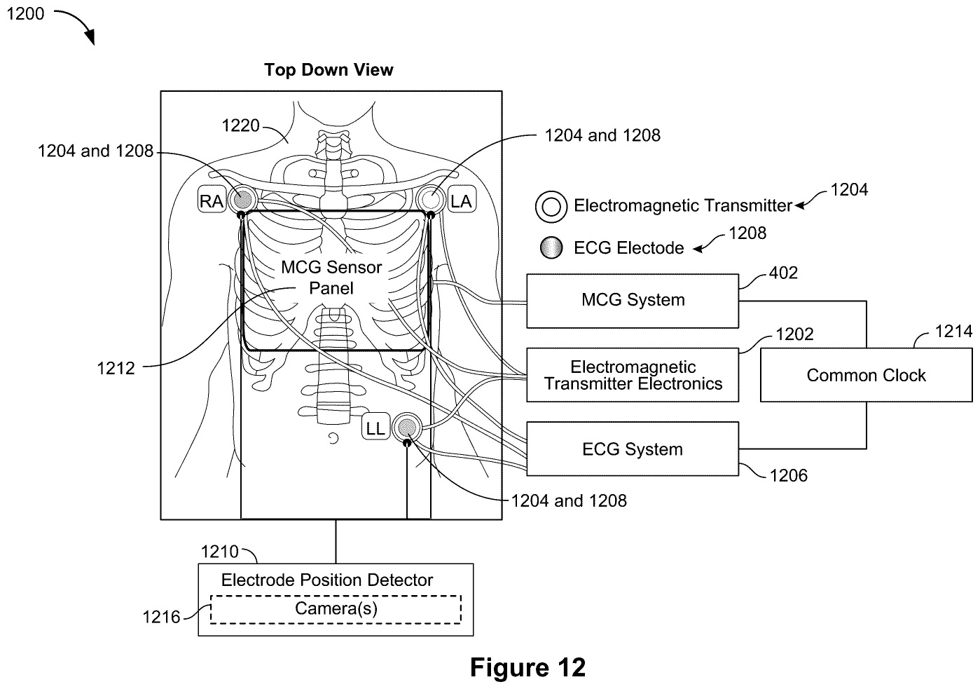

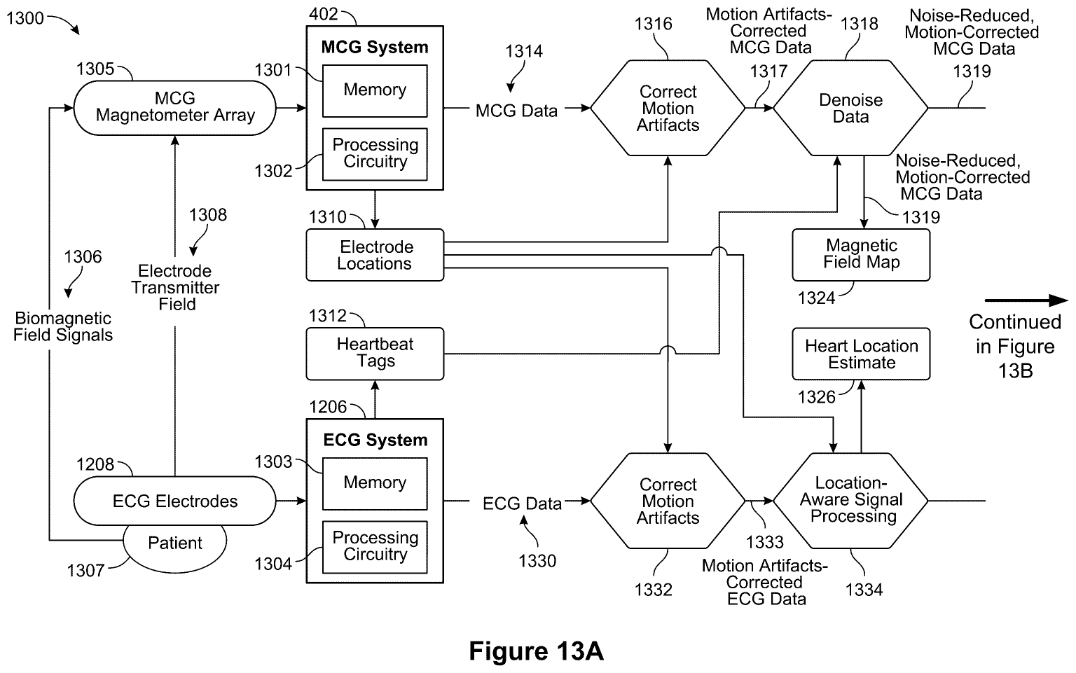

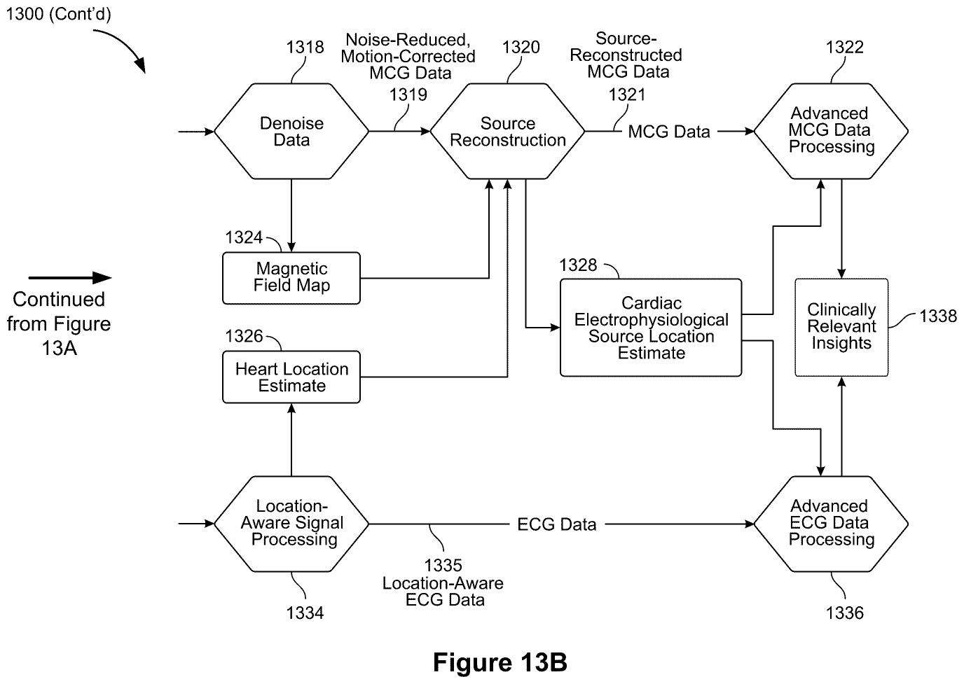

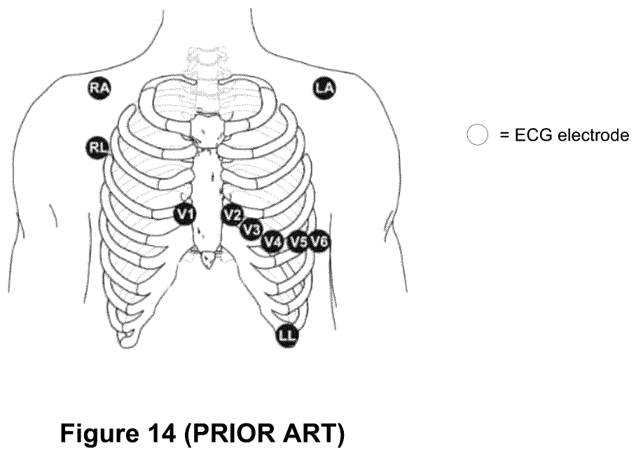

In some embodiments, the MCG system 402 is part of a hybrid system that also includes an ECG system (e.g., ECG system 1206 in FIG. 12). An exemplary hybrid system is described with respect to FIG. 12. The hybrid system enables MCG and ECG data to be simultaneously acquired. In some embodiments, the one or more electromagnetic transmitters 406 comprise a plurality of electromagnetic transmitters, each of which is positioned at a respective ECG electrode (e.g., ECG electrode 1208) (e.g., ECG pad, electrode tracking beacon) of the ECG system. Further details of a hybrid ECG and MCG system are described below with respect to FIGS. 13 and 14. In some embodiments, an MCG system 402 that is outfitted with electromagnetic transmitters 406 that are positioned at the ECG electrodes comprises magnetometers with sufficient dynamic range (e.g., 1 pT to 50 pT) and slew rate (e.g., 10 pT/s, 20 pT/s or 30 pT/s) to capture both the MCG signals from the heart as well as signals from the electrode tracking beacons. In the case of frequency-multiplexed beacon signals, the CPI driving system 408 for the electromagnetic transmitters should be synchronized with the MCG system at the sample rate of the sensors (e.g., a rate of 1000 samples/second is typical) to facilitate the implementation of spectral filtering techniques such as lock-in detection.

FIG. 5A illustrates a magnetic field distribution due to current flowing through a loop of coils in an XY plane, in accordance with some embodiments. In some embodiments, the magnetic field intensity at some vector vec(r) relative to the loop center generated by the loop is represented by:

$$\vec{H} ( r ) = \frac{\vert m \vert}{4 π {r}^{3}} ( 2 \hat{r} cos θ + \hat{θ} sin θ ) \qquad ( 1 )$$

In Equation (1), |m|=NIA for N turns in a loop of coils of cross-sectional area A encircling the z-axis, I is the current in the coil, and θ indicates the polar angle from Z.

In some embodiments, the continuous tracking of the human subject position (relative to the MCG magnetometers) during MCG measurements is implemented via a carrier field H_{c}(t) applied to the coil with an oscillating tone at a frequency that is higher than a frequency range of the biomagnetic field signals. The coil is mounted at a nominal displacement r_{c} from a magnetometer. The coil is mechanically modulated by a small, low-frequency displacement ∂r(t) due, for example, to chest wall dilation. The transfer function from ∂r to ∂H is:

$$\frac{\partial \vec{H} ( t )}{\vert {H}_{c} \vert} = - 3 ( \frac{\partial r ( t )}{{r}_{c}} ) \frac{2 \hat{r} cos θ + \hat{θ} sin θ}{\sqrt{3 {cos}^{2} θ + 1}} \qquad ( 2 )$$

In some embodiments, the magnetometer is a total-field sensor. The signal from the coil that is detected by the total-field sensor in a static ambient field H_{E} has a dimensionless modulation depth of:

$$\frac{\partial B ( t )}{{B}_{c}} = \frac{\partial \vec{H} ( t )}{\vert {H}_{c} \vert} \cdot {\hat{H}}_{E} \qquad ( 3 )$$

where ∂B(t)/B_{c} is the change in time-varying magnetic flux density normalized by the magnetic flux density due to the coil and Ĥ_{E} is the magnitude of the ambient field. Note that only the component of the modulated field that is aligned with H_{E} contributes to the measurement. The modulation depth is proportional to both the magnitude and the alignment of the oscillating component.

FIG. 5B illustrates the dimensionless modulation depth in a case where a current-carrying coil is displaced at a distance r from a magnetometer along the coil axis, in accordance with some embodiments.

In some embodiments, the MCG system 402 is configured to detect, measure, and/or collect, via the array of magnetometers 201, a magnetic field signal produced by the electric current as the electric current travels through the electromagnetic transmitter 406. In some embodiments, the magnetic field signal is a time-varying signal, and the relative amplitude of the signal at each magnetometer position depends on the relative distance and angle between the electromagnetic transmitter and the respective magnetometer. As the electromagnetic transmitter 406 moves, either due to physical movements of the human subject with respect to the patient bed or due to movement of the chest wall of the human subject during a heartbeat or breathing, the carrier frequency amplitude recorded by the magnetometers will change. This amplitude modulation is directly related to the distance and angle between the electromagnetic transmitter 406 and a respective magnetometer 201.

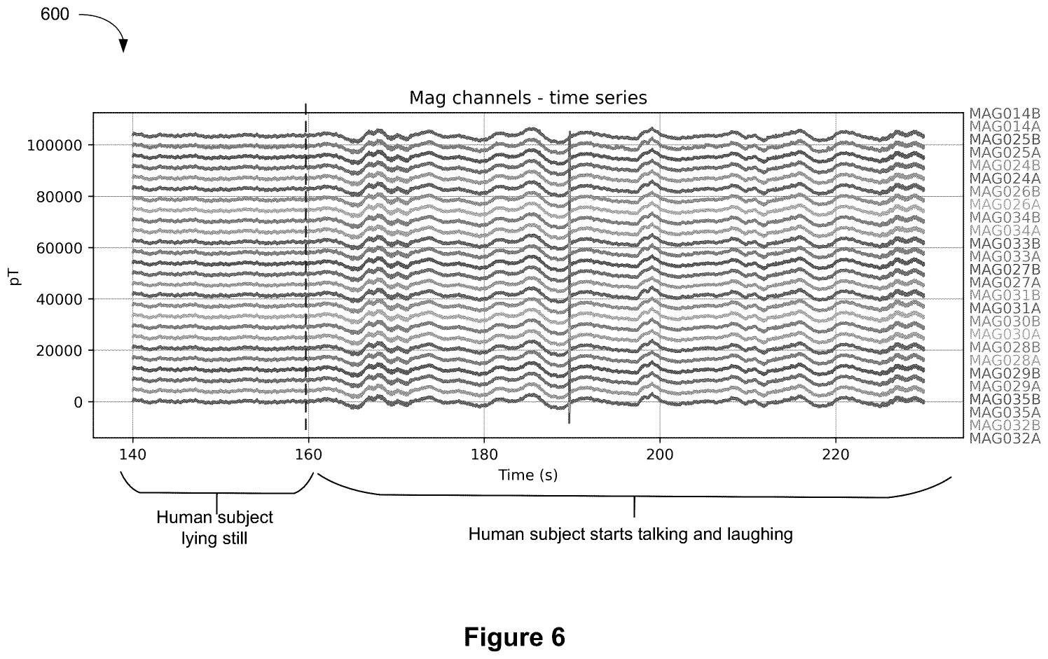

FIG. 6 illustrates a graph 600 showing actual raw time-series magnetic field signals collected by an array of magnetometers by placing the array over a human subject at ambient temperature (e.g., room temperature) and in a magnetically unshielded environment, in accordance with some embodiments. Each of the lines in the graph 600 corresponds to magnetic field signals collected by a respective channel (e.g., a magnetometer) of the array. The lines in the graph 600 have been offset for clarity. At time t=˜140 to 160 s, the human subject was lying still. From around t=˜160 s, the human subject began talking and laughing. The magnetic field data in the graph 700 suggests that talking and/or laughing by a human subject can cause chest movements that in turn result in the presence of large, common mode, low-frequency artifacts in the magnetic field signals. The data in FIG. 6 suggests that if the magnetic field signals shown in FIG. 6 were identified via a CPI coil (e.g., electromagnetic transmitter 406), the artifact signals can be confidently removed from the collected magnetic field signals because of the differences in spectral characteristics between magnetic field signals from the heart when a patient is lying still and magnetic field signals from the heart when the patient exhibits additional movements.

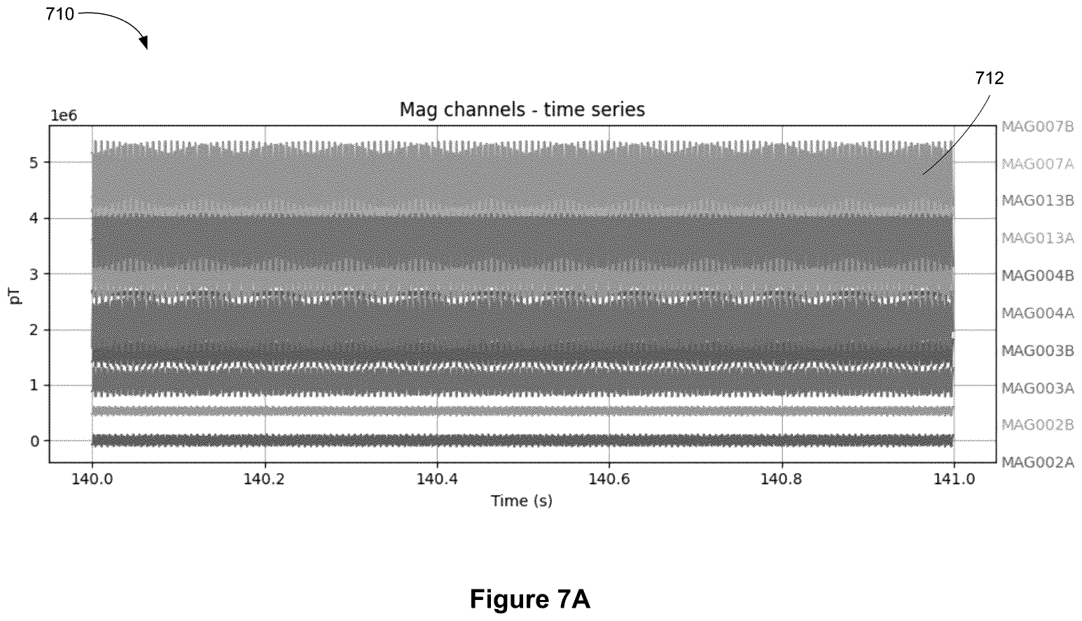

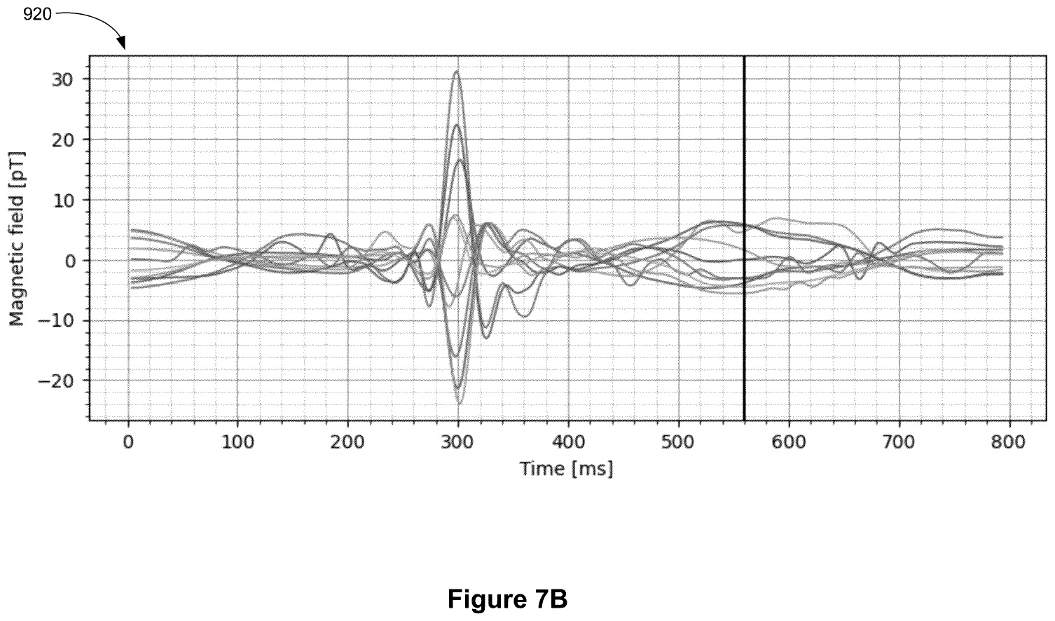

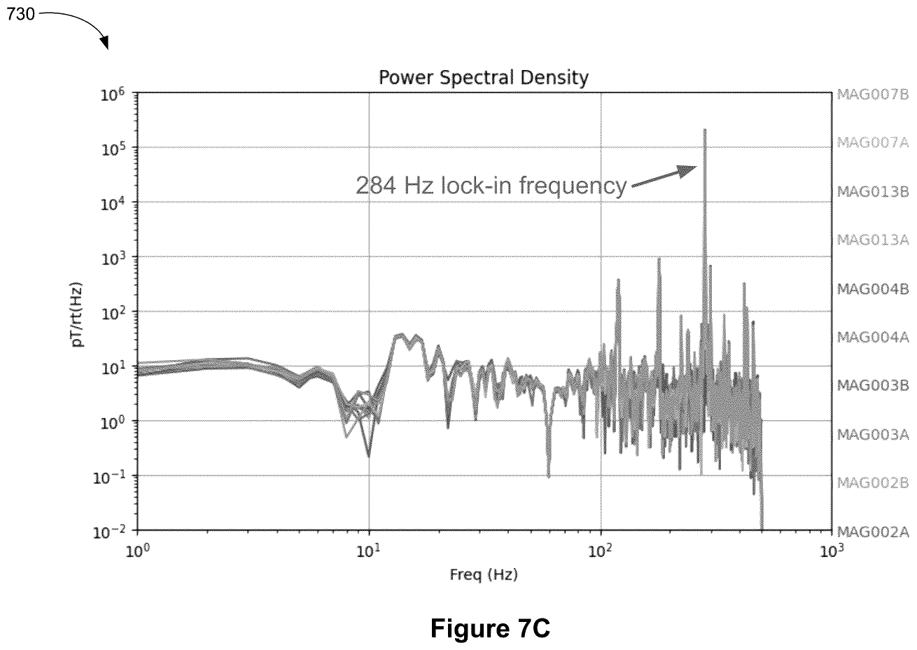

FIG. 7A is a graph 710 showing raw magnetic field signals detected by a multi-channel magnetometer array over a one-second duration, by placing the magnetometer array above the chest of a human test participant whose chest is affixed with a CPI coil, in accordance with some embodiments. Using the raw magnetic field signals 712 from magnetometer 007B as an example, as the coil moves (e.g., due to breathing or chest wall movement of the human subject), the movement of the coil modulates the amplitude of the carrier frequency for the magnetometers (synonymous with amplitude modulation in an AM radio). The magnetic field signals in the graph 710 are dominated by the CPI coil carrier frequency. The carrier frequency manifests with different amplitudes on different magnetometers, reflecting the relationship between coil-sensor distance and magnetic field amplitude. FIG. 7B is a graph 720 showing respective average MCG signals extracted from the raw magnetic field signals in FIG. 7A, for all magnetometer channels, in accordance with some embodiments, demonstrating that the MCG signal is not contaminated by fields from the active CPI coil. FIG. 7C shows a power spectral density 730 of the raw magnetometer signals in accordance with some embodiments. The large peak at 284 Hz in the power spectral density plot 930 corresponds to the carrier signal of the CPI coil.

As illustrated in FIG. 7A, movement of the electromagnetic transmitter 406 (e.g., due to physical movements of the human subject with respect to the patient bed or movement of the chest wall of the human subject due to breathing or the heart beating) modulates the carrier frequency amplitude recorded by the magnetometers. In some embodiments, suitable demodulation (e.g., lock-in amplification or Kalman filtering) of the envelope measurement reveals this motion while preserving it distinctly from the MCG activity. This remains true even if the body motion occurs at the same frequency as the MCG activity. Body motion that spectrally overlaps with biomagnetic cardiac signals is common for many types of physiological motion (e.g., breathing), but is especially problematic for BCG-induced artifacts in MCG data sets, because in that case the cardiac activity occurs simultaneously in both the BCG and MCG signals, and thus cannot be removed or reduced by averaging.

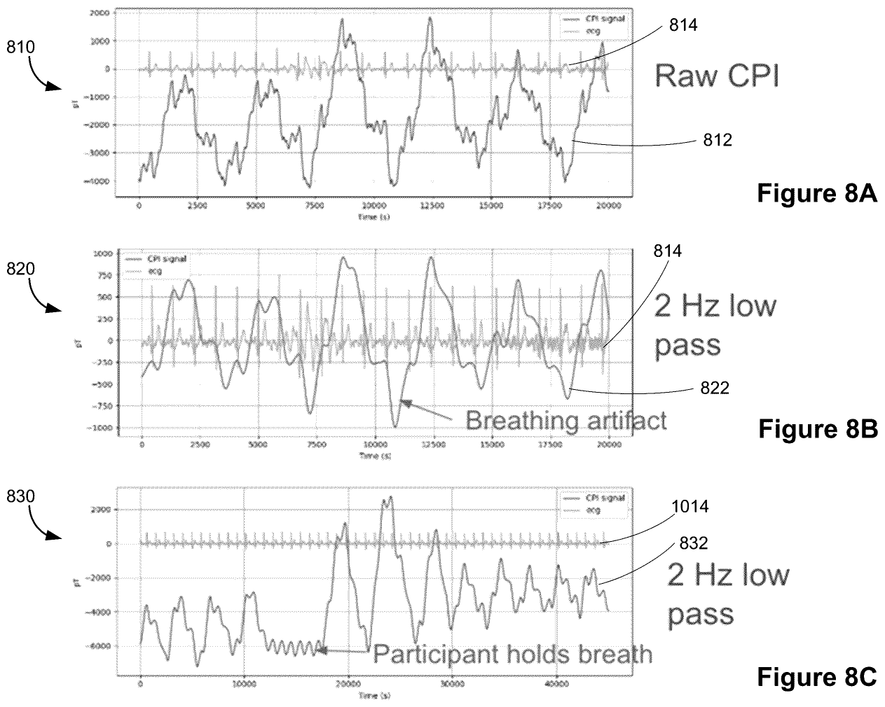

FIG. 8A illustrates a graph 810 that includes a line plot 812 corresponding to unfiltered, demodulated magnetic field signals from the CPI coil, in accordance with some embodiments. The line plot 812 corresponds to a representative segment of the raw magnetic field data in FIG. 7A. The line plot 812 is shown alongside a line plot 814 corresponding to ECG measurements that are simultaneously acquired with the MCG measurements.

FIG. 8B illustrates a graph 820 that includes a line plot 822 corresponding to filtered, demodulated magnetic field signals, in accordance with some embodiments. The line plot 822 is obtained by applying a 2 Hz low-pass filter to the unfiltered, demodulated magnetic field signals in FIG. 8A. The line plot 822 shows breathing artifacts from chest inflation of the test subject.

FIG. 8C illustrates a graph 830 that includes a line plot 832 corresponding to a longer segment of the filtered, demodulated magnetic field data that is shown in FIG. 8B, in accordance with some embodiments. The line plot 832 includes a segment where the participant held their breath.

The examples of FIGS. 6, 8B, and 8C illustrate that magnetic field signals generated by a CPI coil are highly sensitive to, and are indicative of, movement of the human subject (e.g., with respect to the patient bed or chest wall movements). Thus, in some embodiments, the time-series magnetic field signals from the CPI coil can be used to determine artifacts in MCG data due to movement of the human subject (e.g., motion artifacts), such as physical movement of the human subject with respect to the patient bed, or breathing, or coughing. The motion artifacts can be identified in the CPI coil magnetic field data by their temporal characteristics. For example, in some embodiments, the motion artifacts have magnetic field signal contributions with spectrally dominant low-frequency components (e.g., less than 1 Hz). In some embodiments, the magnetic field signals due to motion artifacts can be separated from the magnetic field signals of interest (e.g., from the heart) using spectral filtering.

In some embodiments, the magnetic field signals due to motion artifacts can be separated from the magnetic field signals of interest by using magnetic field signals from the motion artifacts as regressors, as well as inform spatially dependent denoising techniques (for example to spatial classifiers for blind-source-separation methods) and cardiac source reconstruction algorithms (e.g., by providing time intervals of expected stable source positions should increase confidence in reconstruction).

In some embodiments, the time-series magnetic field signals from the CPI coil can be used to determine “smaller” movements by the patient, such as movement of the chest wall due to the heart beating. The spectral content of these movements tends to be higher compared to the spectral content of the motion artifacts (e.g., physical movement of the human subject with respect to the patient bed, or breathing, or coughing), and time-locked to the heartbeat compared to that from the motion artifacts.

In some embodiments, to boost the signal-to-noise ratio of the magnetic field signals generated by the coil, lock-in amplification is performed at a respective magnetometer channel based on the known driving frequency of the coil, to extract the signal amplitude at each magnetometer location. The basic principle of lock-in amplification is to multiply a noisy signal by a reference sinusoid at the frequency of interest, and integrate over a predefined time period (e.g., usually a few milliseconds to a few seconds). At each time point, the magnetic inverse problem can be solved to estimate, using the CPI tone amplitude, the coil's continuous position in space relative to the magnetometer array.

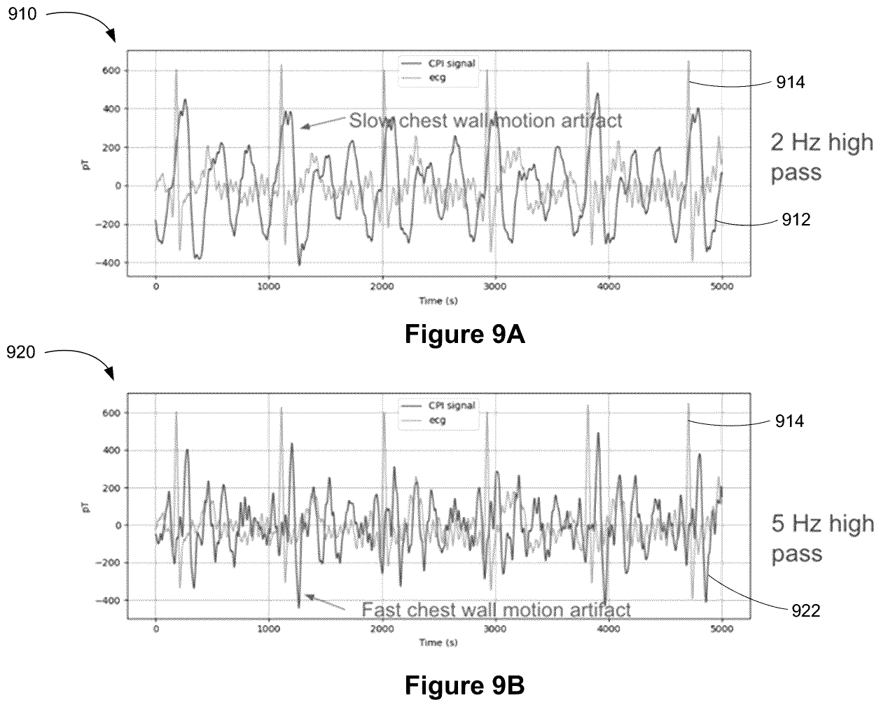

In some embodiments, the seismocardiogram measurements can be used with a correlated signal processing technique, to strip the ballistocardiogram artifact signals from the MCG data. Ballistocardiogram is the recording of whole body movement resulting from recoil due to fluid ejection from the heart. Seismocardiogram is the vibration of the chest wall due to the cardiac muscle contraction. In some embodiments, the ballistocardiogram signals can be distinguished from the seismocardiogram by applying signal processing techniques. For example, FIGS. 9A and 9B illustrate respective graphs that are obtained by applying different filtering parameters to the CPI coil magnetic field data. FIG. 9A is a graph 910 showing a demodulated and filtered magnetic field signal 912 generated by a CPI coil that is affixed to a human subject, after application of a 2 Hz high-pass filter to the raw CPI magnetic field signals. The filtered CPI magnetic field signal 912 reveals features akin to the ballistic, oscillatory motion of the CPI coil and the body of the test subject due to chest wall compressions (ballistocardiogram), as evidenced by time-locking the filtered CPI magnetic field signal 912 with the ECG data 914. FIG. 9B is a graph 920 showing a filtered CPI magnetic field signal 922 that is generated by applying a 5 Hz high-pass filter to the same CPI magnetic field signals for FIG. 9A. The higher frequency filter parameters reveal more rapid motion that may be due to the chest wall compression, akin to a seismocardiogram.

In some embodiments, the ballistocardiogram signals can be distinguished from the seismocardiogram by utilizing two CPI coils, in which one of the coils is placed over the heart while the other coil is positioned further away from the heart. The coil that is positioned further away from the heart will be more sensitive to the BCG than the seismocardiogram. For example, an electromagnetic transmitter that is placed on the chest detects both the seismocardiogram and ballistocardiogram signals, whereas an electromagnetic transmitter that is placed elsewhere, such as on the shoulder, would detect only ballistocardiogram signals.

According to some embodiments of the present disclosure, the ability to remove ballistocardiogram artifact signals from the MCG data enables MCG systems equipped with the current disclosure to be deployed to many environments, including environments that are magnetically unshielded, have high magnetic stray field signals, or in environments where patients have to be scanned in ordinary exam rooms and on ordinary patient beds, including those with steel bed frames. This is because the BCG can lead to movement of equipment in contact with the patient body, such as steel frame beds, that would generate magnetic field artifacts in the MCG data that are time locked to the patient's heartbeat.

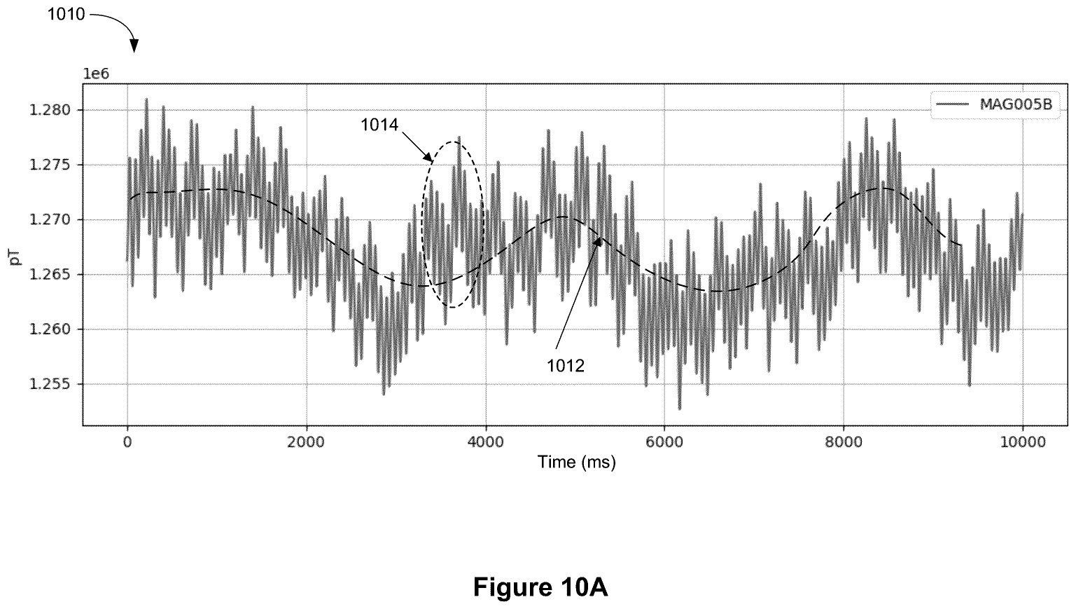

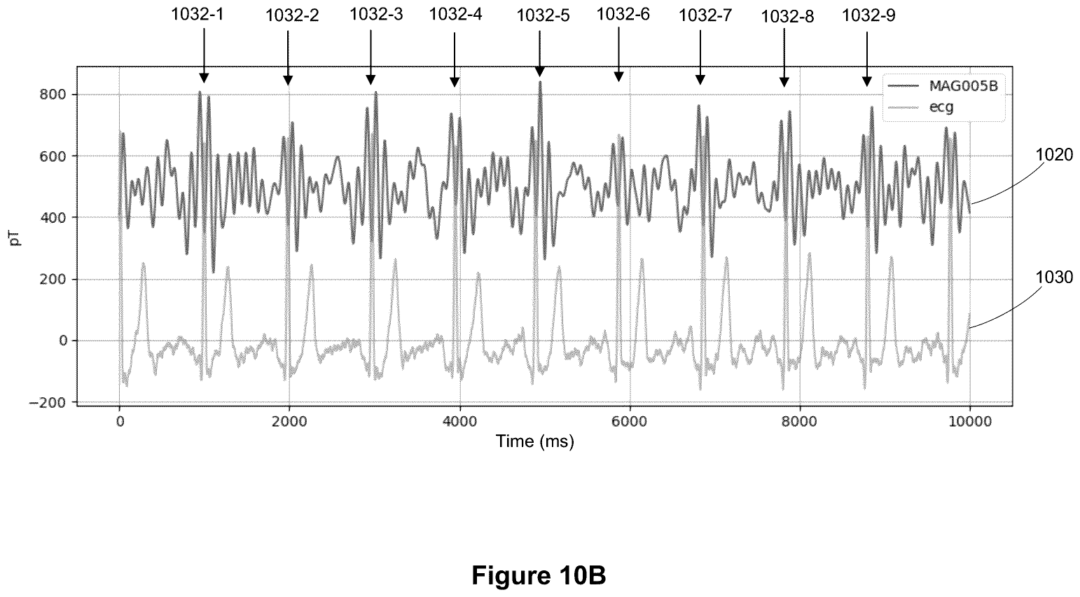

In some embodiments, magnetic field signals emitted from a CPI coil (e.g., electromagnetic transmitter 406) that is affixed to a human subject can provide seismocardiogram (chest wall motion) measurements. This is illustrated in the example of FIG. 10A and FIG. 10B.

FIG. 10A is a graph 1010 showing variations in amplitude of a demodulated CPI signal from a single magnetometer, in accordance with some embodiments. The signal amplitude in the graph 1010 is detected by a multi-channel magnetometer array at room temperature and in a magnetically unshielded environment, by placing the magnetometer array over a human test subject affixed with a CPI coil (electromagnetic transmitter 406) over their heart. The graph 1010 is obtained by filtering the raw magnetic signals around the modulation frequency, and then performing a continuous software lock-in. The CPI coil emits a tone that has a higher frequency than a frequency of the biomagnetic signals emitted from the human heart. As used herein, the frequency of the CPI coil is also referred to as carrier frequency. Graph 1010 shows that the magnetometer array is capable of detecting magnetic field signals generated by both the CPI coil and the test subject's heart. In the graph 1010, both low-frequency components (indicated by trace 1012) and high frequency components (indicated by ellipse 1014) are visible as the coil moves due to breathing and smaller chest wall oscillations of the test subject.

In some embodiments, seismocardiogram (e.g., chest wall motion, or vibration of the chest wall due to the cardiac muscle contraction) measurements can be obtained via filtering and analysis of time-series magnetic field signals generated by a CPI coil. FIG. 10B is a graph 1020 of the same modulated magnetic field data in FIG. 10A (i.e., graph 1010), after application of a filter to remove low-frequency content (e.g., content that is less than 2 Hz) due to movement of the patient, breathing signals, and DC offsets. FIG. 10B also shows an electrocardiogram 1030 of the human test subject that is measured using electrocardiography (ECG) at the same time as the MCG measurements. A comparison of the graph 1020 with the electrocardiogram 1030 reveals a periodic, damped oscillation that is time-locked to the heartbeat of the test subject, indicated by arrows 1032-1 to 1032-9. The data in FIG. 10B shows that CPI signals corresponding to chest wall movements of a human subject from the heartbeat can provide seismocardiogram measurements, with the graph 1020 being the seismocardiogram. Stated another way, some embodiments of the present disclosure utilize a frequency multiplexing approach (e.g., combining multiple signals with different frequencies in a single MCG channel) that preserves the biomagnetic field signals while enabling integrated, synchronized, continuous measurement of the seismocardiogram.

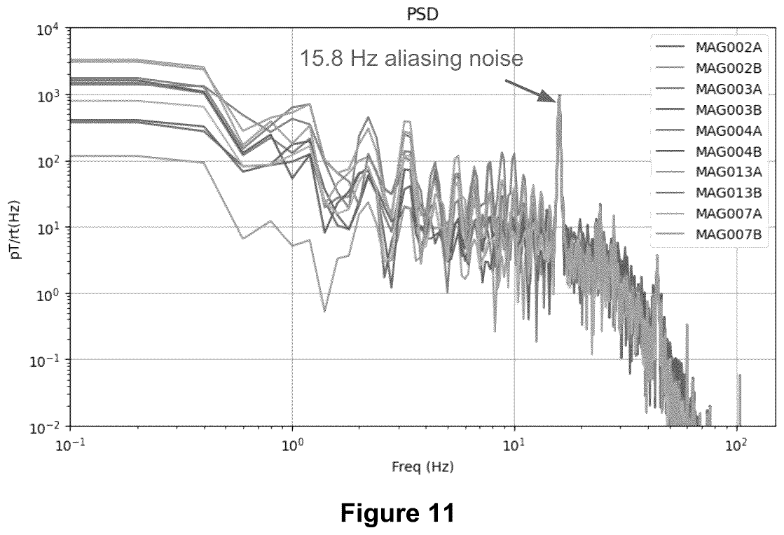

FIG. 11 illustrates a power spectral density plot of the demodulated CPI coil signal of FIG. 8A, in accordance with some embodiments. The complex spectral content represents the motion of the coil. The peak at 15.8 Hz is an aliasing artifact.

In some embodiments, the setup 400 can also aid an operator in the initial positioning of the MCG sensor array during MCG device setup, so as to produce more repeatable and systematic recordings. In particular, because the MCG signal quality is highly dependent on the positioning of the MCG sensor array, the electromagnetic transmitter 406 can provide the operator with live positional feedback to position the MCG array in a location and orientation that ensures coverage of cardiac magnetic signals.

### Combined ECG and MCG System for Continuous Co-Localized Electrophysiology Measurements

Some embodiments of the present disclosure are directed to a MCG system that is configured to acquire MCG data from a human subject, receive electrocardiography (ECG) data from an ECG system that is communicatively connected to the MCG system, and integrate the MCG data with the ECG data to obtain continuous co-localized electrophysiology measurements of a human subject. In some embodiments, the MCG system and the ECG system are part of a combined (e.g., hybrid) system that is configured to acquire both ECG and MCG data.

While pervasive in its application for diagnosing and monitoring cardiac function, ECG lacks a suitable method to localize electrode locations on the human subject's body and co-register those locations to the position of the subject's heart. Meanwhile, MCG typically suffers from low signal-to-noise ratio due to environmental interference. The limitations of each system can affect its ability to effectively localize cardiac electrical activity, which can aid disease diagnosis.

For ECG, the inverse problem is made technically difficult due to uncertainties associated with the inherent lack of knowledge of exact tissue conductivity, locations of ECG electrodes, and adherence level to skin. Furthermore, because an ECG measurement generally uses no more than 15 ECG electrodes placed only the front and one side of a subject's torso, the spatial resolution of body surface potential measurements (i.e., the number of locations on the body that are sampled based on the number of ECG electrodes used) is typically too low to retrieve complete, three-dimensional reconstruction of heart electrophysiology.

In MCG, the inverse problem to find cardiac sources is typically solved using methods like L2 minimum norm estimation (e.g., borrowing well-developed techniques from magnetoencephalography). Since magnetic fields propagate through body tissue with minimal distortion, properly-designed MCG systems perform relatively well on the task of source localization, compared to ECG with similar channel counts that lack an accurate conductivity model for the patient's specific anatomy. However, the source inversion performance of MCG systems remains hindered by operational challenges such as participant movement artifacts, and magnetic noise. In particular, MCG is susceptible to motion artifacts in the data, which not only increases noise but also degrades the stability of functional source inferences.

According to some aspects of the present disclosure, the limitations on the ECG and MCG systems can be addressed by a combined (e.g., hybrid) ECG and MCG system that concurrently acquires data with both modalities.

For example, in some embodiments, the unknown ECG electrode locations can be inferred by the MCG system using an appropriate magnetic tracking scheme, such as the electromagnetic transmitter(s) 406 that are described with respect to FIG. 4 and FIG. 12. In some embodiments, by using MCG methods to localize the heart location, the locations of the ECG electrodes can be determined relative to the heart itself, thereby providing key anatomical information about the position and orientation of the cardiac sources within the chest cavity and enabling inference of the conductivity of the intermediate tissue.

In some embodiments, the limitation of low signal-to-noise ratio in an MCG system can be addressed via averaging of multiple heartbeats together, which is most precisely done with clear timing indications for each R-wave. The ECG system provides the ideal trigger for this purpose. Moreover, since MCG is a non-contact technique, artifacts from patient movements do not typically have proxies to aid in regression and removal. By combining the time resolved ECG locations, MCG data can be more effectively denoised.

Combining data from a MCG system with data from an ECG system can provide complete and continuous relative positioning information of the ECG electrodes and a patient's heart. The hybrid system can provide cardiologists with detailed electrophysical information about the patient's cardiac health, by offering continuous, complete information about the ECG electrode locations during a measurement. The hybrid system can also provide patient anatomy data via the ECG electrode positions in three-dimensional space, or correct for electrode misplacements by operators. Cardiologists may require ECG electrode placement information (or misplacement information) when investigating detailed electrophysiology without requiring patients to undergo invasive procedures such as angiograms.

FIG. 12 is a schematic diagram of a system 1200 for performing MCG and ECG measurements, in accordance with some embodiments. The system 1200 includes a MCG system 402 and an ECG system 1206 that are communicatively connected with each other.

In some embodiments, both the MCG system 402 and the ECG system 1206 are part of the system 1200. In this scenario, the system 1200 is also referred to a hybrid system, a combined system, or a combined ECG-MCG (or MCG-ECG) system.

In some embodiments, the MCG system 402 is part of the system 1200 whereas the ECG system 1206 is not part of the system 1200. In some embodiments, the ECG system 1206 is located in the same room as the system 1200. The MCG system 402 is communicatively connected with the ECG system 1206 and receives ECG data from the ECG system 1206.

In some embodiments, the ECG system 1206 is part of the system 1200 whereas the MCG system 402 is not part of the system 1200. In some embodiments, the MCG system 402 is located in the same room as the system 1200. The ECG system 1206 is communicatively connected with the MCG system 402 and receives MCG data from the MCG system 402.

Referring to FIGS. 12 and 13A, in some embodiments, the MCG system 402 includes memory 1301 (e.g., memory 206), processing circuitry 1302 (e.g., CPU(s) 202 or processors), and a plurality of magnetometers (e.g., magnetometers 201). The MCG system 402 is configured to, during use of the system 1200, measure biomagnetic field signals of a human subject 1220.

In some embodiments, the MCG system 402 includes a MCG sensor panel 1212 on (or within) which magnetometers are mounted. In some embodiments, the MCG sensor panel 1212 comprises a rigid panel. In some embodiments, the position of a respective magnetometer relative to other magnetometers on the MCG sensor panel 1212 is known. In some embodiments, the magnetometers on the MCG sensor panel 1212 are arranged in an array (e.g., array 404).

In some embodiments, the ECG system 1206 includes memory 1303, processing circuitry 1304 (e.g., CPU(s) or processors), and one or more ECG electrodes 1208 that are mounted on a human subject 1220 during ECG measurements. The ECG system 1206 is configured to measure ECG data comprising cardiac electrical activity of the human subject 1220 (via the one or more ECG electrodes 1208).

In some embodiments, the system 1200 includes an electrode position detector 1210 that is configured to determine location information (e.g., positional information) of the one or more ECG electrodes 1208 that are mounted on the human subject 1220. In some embodiments, the electrode position detector 1210 is distinct from the MCG system 402. In some embodiments, the electrode position detector 1210 is distinct from the ECG system 1206. In some embodiments, the location of a respective electrode is expressed in terms of coordinates, or a combination of distances and/or angles.

In some embodiments, the location information of the one or more ECG electrodes 1208 comprises relative location information (as opposed to absolute location information). For example, the location information of the ECG electrodes comprises positional information of a respective electrode relative to the plurality of magnetometers 201, or relative to a predefined anatomical position of the human subject 1220, or relative to a target organ of the human subject 1220. In some embodiments, the location information of the one or more ECG electrodes 1208 are determined on a coordinate system of the MCG system 402, or on a common coordinate system that is common to both the ECG system and the MCG system.

In some embodiments, the electrode position detector 1210 is configured to determine the location information of the one or more electrodes at a sampling rate that matches or exceeds a rate of movement from the human subject during examination, for at least a substantial portion of the duration of measurement of the human subject.

In some embodiments, the electrode position detector 1210 includes one or more imaging cameras 1216. The electrode position detector 1210 is configured to determine the location information of the one or more electrodes that are mounted on the human subject using optical tracking (e.g., via one or more cameras 1216), and co-register the optical location information with the position of the subject's heart (e.g., determined from the biomagnetic field signals). The one or more cameras 1216 also determine the location of the MCG system 402 (e.g., via fiducial markers).

In some embodiments, each ECG electrode of the one or more ECG electrodes 1208 is physically coupled to (e.g., affixed to) a respective acoustic beacon that emits a unique, distinct acoustic signature pulse. The electrode position detector 1210 is configured to identify the source of the respective acoustic beacon, and hence the electrode on which the respective acoustic beacon is affixed to, based on the unique acoustic pattern. Accordingly, the electrode position detector 1210 can determine the location information of the one or more electrodes by measuring the acoustic signals emitted from the one or more acoustic beacons and co-registering the location information with the position of the subject's heart (e.g., that is determined from the biomagnetic field signals).

In some embodiments, co-registering the location of the ECG electrodes with the position of the subject's heart includes recording the locations of the ECG electrodes in a coordinate system of the MCG sensor panel 1212 geometry.

In some embodiments, the system is configured to enhance the measured MCG data and/or ECG data in accordance with the determined location information of the one or more ECG electrodes 1208. This is explained in further detail with respect to FIGS. 13A and 13B.

With continued reference to FIG. 12, in some embodiments, the system 1200 includes one or more electromagnetic transmitters 1204 (e.g., electromagnetic beacons). The electromagnetic transmitters 1204 are coupled to an electromagnetic transmitter electronics system 1202. In some embodiments, the electromagnetic transmitter electronics system 1202 is configured to drive a respective current (e.g., an AC current) to a respective electromagnetic transmitter 1204. The respective electromagnetic transmitter 1204 generates a magnetic field as a result of current flowing through the electromagnetic transmitter 1204, which is detected by the array of magnetometers (and described previously with respect to FIGS. 4, 5A, and 5B).

In some embodiments, the electromagnetic transmitters 1204 have features that are similar to those found in the electromagnetic transmitter 406. In some embodiments, the electromagnetic transmitter electronics system 1202 has features similar to those found in the CPI driving system 408. These features are not repeated for the sake of brevity.

In some embodiments, as illustrated in FIG. 12, each of the ECG electrodes 1208 is physically coupled to (e.g., affixed to, such as using glue or other adhesives) a respective electromagnetic transmitter 1204. In this case, the MCG system 402 is the electrode position detector and is configured to determine the location information of the one or more electrodes by measuring, via the magnetometers 201, magnetic field signals emitted from the one or more electromagnetic transmitters 1204. Because the relative amplitude of the magnetic field signal at each magnetometer position depends on the relative distance between a respective electromagnetic transmitter 1204 and the respective magnetometer, the MCG system 402 (via processing circuitry 1302) may thus accurately infer (e.g., determine) the offset distance—and dynamically changing distance—between the ECG electrodes 1208 and its own coordinate system.

In some embodiments, each of the electromagnetic transmitters 1204 is configured to emit a unique electromagnetic frequency.

In some embodiments, the MCG system 402 is configured to, during use of the system 1200, track the respective positions of the ECG electrodes 1208 by acquiring electromagnetic emission (e.g., magnetic field signals) from the ECG electrodes 1208, thereby obtaining continuous, relative positioning information of the ECG electrodes relative to the MCG sensor panel 1212.

In some instances, one or more of the ECG electrodes 1208 may move during use of the system 1200, either due to patient movement, sliding of the electrodes, or movement of the patient's chest wall during a heartbeat or breathing. This data (e.g., motion data) is recorded alongside the MCG data (i.e., the movements are reflected in the magnetic field data recorded by the MCG system 402). As a result, the system 1200 can track each ECG electrode via its corresponding electromagnetic transmitter's signal, by determining the respective ECG electrode's location and orientation at each point in time. In the case of electrodynamic beacons (e.g., transmitters), this proceeds by solving the magnetic inverse problem. For example, the inverse problem can be solved in any number of ways, including linear regression, L2 minimum norm estimation, beamforming, and Markov chain Monte Carlo (MCMC) methods.

In some embodiments, the disclosed system 1200 with electrode tracking beacons (comprising electromagnetic transmitters 1204 integrated with ECG electrodes 1208) are advantageous over other combined ECG-and-MCG systems because they do not require any new detection hardware such as camera(s) or LIDAR or microwave imaging system, since the MCG sensor panel 1212 is capable of “imaging” (e.g., detecting) the electrode tracking beacons.

In some embodiments, to avoid electromagnetic “contamination” of the MCG data (e.g., due to the presence of biomagnetic field signals from cardiac activity and magnetic field signals from the electromagnetic transmitters), the magnetic field signals from the electrode tracking beacons can be separated from the biomagnetic field signals by either time or frequency multiplexing.

In the case of time multiplexing, segments of “ECG pad imaging” (e.g., measuring the magnetic field signals from the electrode tracking beacons, during activation of the electromagnetic transmitters) can be interleaved with “MCG data acquisition.” For example, in some embodiments, the system 1200 is configured to measure biomagnetic field signals from cardiac activity while the electromagnetic transmitters are deactivated (e.g., by turning off the current supply to the electromagnetic transmitters). In some embodiments, time-multiplexing can be used during the ECG setup to ensure that the ECG electrodes are accurately placed on the patient.

In the case of frequency multiplexing, the magnetic field signals from the electrode tracking beacons are kept spectrally distinct from cardiac activity. Frequency multiplexing allows filtering methods to be applied on the data, and can be useful for recording continuous ECG electrodes location data during a MCG examination.

In some embodiments, data collected by the ECG system 1206 and the MCG system 402 are appropriately time-synchronized. FIG. 12 illustrates that in some embodiments, the ECG system 1206 and the MCG system 402 share a common clock 1214 (e.g., common time base). The common clock 1214 is configured to generate a common clock signal to drive or otherwise control the ECG system 1206 and the MCG system 402 such that the ECG electrode positions can be correlated to artifacts in the ECG/MCG data.

In some embodiments, the system 1200 is configured to evaluate a health condition of a human subject 1220 (or any a living subject) during use of the system 1200. For example, the system 1200 is in use when at least one of the conditions is met: (1) the one or more ECG electrodes 1208 are mounted on a human subject 1208 and are activated, (2) the MCG system 402 is activated, (3) the MCG sensor panel 1212 is in proximity to the human subject 1208, (4) the MCG sensor panel 1212 is in proximity to the electromagnetic transmitters 1204, or (5) the magnetometers of the MCG system 402 have known positions relative to one another.

In some embodiments, the system 1200 is configured to enhance the measured MCG data in accordance with the measured MCG data and the determined location information of the one or more ECG electrodes 1208.

In some embodiments, the system 1200 is configured to enhance the measured ECG data in accordance with the measured MCG data and the determined location information of the one or more electrodes.

In some embodiments, the system 1200 is configured to perform an overall health assessment of the living subject based on integration of the measured MCG data and the measured ECG data.

In some embodiments, the tracked locations of the electromagnetic transmitters can be fed into an MCG analysis pipeline and an ECG analysis pipeline, to obtain clinically relevant insights about a patient. FIGS. 13A and 13B collectively illustrate a data flow process 1300 for a system that includes an ECG system 1206 and a MCG system 402, in accordance with some embodiments. In some embodiments, the steps of the process 1300 are performed by respective processing circuit(ries) of the MCG system 402 and the ECG system 1206. In some embodiments, the steps of the process 1300 are collectively performed by one or more processors (e.g., CPU(s) 302) of a computer system (e.g., computer system 120) that is communicatively connected with both the ECG system 1206 and a MCG system 402. In some embodiments, the MCG system 402 corresponds to MCG system 200 (e.g., device 200) and is communicatively connected to ECG system 1206, and the steps of the process 1300 are performed by one or more processors (e.g., CPU(s) 202) of the apparatus 200.

FIG. 13A illustrates that in some embodiments, the MCG system 402 obtains (e.g., acquires, receives, collects, records) magnetic field signals via a MCG magnetometers array 1305 (e.g., MCG sensor panel 1212 and magnetometers 201) that is coupled to the MCG system 402 For example, in some embodiments, the MCG system 402 measures, via the MCG magnetometer array 1305, biomagnetic field signals 1306 from a patient 1307 (e.g., human subject 1220). The MCG system 402 measures, via the MCG magnetometer array 1305, electrode transmitter field signals 1308 generated by ECG electrodes 1208 mounted on the patient 1307.

In some embodiments, the MCG system 402 determines (e.g., via processing circuitry 1302) the locations of the ECG electrodes (electrode locations 1310) based on the biomagnetic field signals 1306 and the electrode transmitter field signals 1308. For example, the processing circuitry 1302 may perform processes such as synchronizing (e.g., temporal synchronization), filtering, lock-in amplification, magnetic field inversion, and/or other processing of the biomagnetic field signals 1306 and the electrode transmitter field signals 1308, to extract the electrode locations 1310.

In some embodiments, the MCG system 402 corrects motion artifacts (1316) in the MCG data 1314 using the electrode locations 1310 (e.g., electrode positional information) to obtain motion artifacts-corrected MCG data 1317. Specifically, the electrode locations 1310 can provide regressors for motion artifacts correction (1316), as well as positional estimation bounds for the heart source currents. The electrode locations 1310 can be used as regressors in the MCG data 1314 because the motion of the ECG electrodes is expected to correlate directly to magnetic artifacts in the magnetic field signals, up to a linear scale factor. Therefore, the time series positional information of the ECG electrodes can be linearly regressed from the MCG data. For example, in some embodiments, the regression coefficient between these two time-series data (e.g., time series ECG information data and time-series magnetic field signals data) can be determined via mean scaling, median scaling, least-squares method, principal component analysis (PCA), or any other possible regression algorithms.

Referring again to FIG. 13A, in some embodiments, the ECG system 1206 obtains ECG data 1330 associated with electrical activity of the heart of the patient 1307 via the ECG electrodes 1208. The ECG system 1206 determines (e.g., via processing circuitry 1304) the timing of individual heartbeats of the patient (heartbeat tags 1312) (e.g., using the peak of the R-wave). In some embodiments, the ECG system 1206 performs synchronization, filtering and other processing (such as threshold detection, or peak detection) to extract heartbeat tags 1312.

In some embodiments, the motion artifacts-corrected MCG data 1317 undergoes a denoising process (denoise data 1318), to obtain noise-reduced motion-corrected MCG data 1319. According to some embodiments of the present disclosure, the denoising process improves the signal-to-noise ratio of the MCG data by averaging multiple heartbeats together. Spectra from multiple heartbeats can be precisely averaged using clear timing indications from the peak of the R-wave that is obtained from heartbeat tags 1312.

In some embodiments, the noise-reduced motion-corrected MCG data 1319 can be used to generate a magnetic field map 1324 that visually depicts a magnetic field spatial distribution of the heart of the patient 1307.

FIG. 13A also shows that in some embodiments, information about the ECG electrode locations 1310 can be fed into the ECG data stream to correct motion artifacts (1332) in the ECG data 1330, thereby obtaining motion artifacts-corrected ECG data 1333. For example, motion artifacts in the ECG data can arise due to movement of the ECG electrodes due to movement of the patient, or sliding of the electrodes due to them not being securely attached on the patient, or movement of the patient's chest wall due to heartbeats or breathing.

In some embodiments, the ECG system 1206 performs location-aware signal processing 1334 based on the motion artifacts-corrected ECG data 1333 and the information about the electrode locations 1310. As used herein, the term “location aware signal processing” refers to analyses that use the electrode positions. For example, in some embodiments, location aware signal processing includes estimating the location of the heart relative to the electrodes (e.g., electrode pads or pads) and magnetometers, and making corrections to lead signal interpretations. A “lead” is a derived representation of electrical activity in the heart, acquired through the weighted combination of signals from two or more ECG electrodes. Consequently, lead signals include “location-awareness” information since they depend on the locations of the electrodes. In conventional systems, the ECG electrode locations are assumed based on standard protocol practiced by the technician. Since the lead signals are dependent on electrode placement, using ECG data to estimate heart location is critically dependent on the correct interpretation of the lead. Knowing the electrode location can correct the definition of the leads.

In some embodiments, location aware signal processing includes using actual directions and/or orientations of the ECG electrodes to determine appropriate scale factors for the electrode signals in deriving leads. In some instances, the standard derivation obtained using assumed/estimated ECG electrode locations can be incorrect, resulting in negative consequences such as false diagnoses and/or further unnecessary cardiovascular testing. The various embodiments disclosed herein eliminate these potential problems by determining the actual locations of the ECG electrodes and their variations during the ECG and MCG measurements, which in turn enable datasets to be validated, and/or leads to be re-derived if needed.

In some embodiments, location aware signal processing includes segmenting data analysis based on data quality determined by when electrodes are stationary. For example, data collected from stationary ECG electrodes is expected to be of better quality than data collected from moving electrodes. In some embodiments, the system 1200 determines (e.g., identifies), from the ECG data, segment(s) of the ECG data corresponding to time intervals where the respective locations of the plurality of electrodes remain unchanged, and estimates a location of the heart of the human subject based on these segment(s).

In some embodiments, location aware signal processing includes determining conductivity corrections based on body thickness estimates (e.g., thickness of the torso) made possible by ECG electrode locations. For example, having a set of known points (ideally corresponding to landmark positions) on the body surface can provide a three-dimensional estimate of the torso size. FIG. 14 is an image illustrating standard placement locations for ECG electrodes (e.g., 10 electrodes for 12-lead ECG), in accordance with some embodiments. In the example of FIG. 14, there is significant torso coverage by the electrodes, and the relative spacings between the electrodes will depend on the body size. Moreover, since V5 and V6 go around the edge of the body, it provides a curvature and thus some torso circumference could be inferred. According to some embodiments of the present disclosure, the known locations of the ECG electrodes (e.g., relative to the MCG array) can be used to estimate the thickness of the torso of the patient and determine conductivity corrections, thus yielding more accurate results than relying on body mass index (BMI) measurements to estimate the thickness of the body.

With continued reference to FIGS. 13A and 13B, in some embodiments, data obtained from the location-awareness signal processing step 1334 can be used to estimate a heart location (1326) of the patient.

In some embodiments, the combination of noise-reduced, motion-corrected MCG data 1319, the magnetic field map 1324, and/or the heart location estimate 1326 can be used for source reconstruction 1320, to derive (e.g., determine) a magnetic source of the biomagnetic field signals. For example, in some embodiments, the system 1200 enables source localization of the cardiac activity to be inferred by a model that uses both (i) MCG data with its MCG array geometry and (ii) ECG data (with tracking information of the ECG electrodes) that is co-registered to the patient and to the MCG array geometry. In some embodiments, the model applies joint denoising techniques to obtain clinically relevant insights from the ECG and MCG data.

In some embodiments, the process 1300 includes estimating (e.g., determining) a cardiac electrophysiological source location 1328 based on the magnetic source of the biomagnetic field signals. For example, estimating the cardiac electrophysiological source location 1328 can include estimating the current density distribution of the heart, and/or approximating the current density distribution of the heart made via electric current dipole reductions.

In some embodiments, advanced MCG data processing 1322 can be performed on the source-reconstructed MCG data 1321 (and/or the noise-reduced, motion-corrected MCG data 1319) and the cardiac electrophysiological source location estimation 1328 data. Advanced MCG data processing refers to denoising algorithms, visualizations, and feature extraction algorithms that may be informed by ECG electrode locations and heartbeat tags. The visualizations and features extracted from this advanced processing can be used to generate clinically relevant insights 1338, such as diagnostic probabilities and source localizations.

In some embodiments, advanced ECG data processing 1336 can be performed on the location-aware ECG data 1335 and the cardiac electrophysiological source location estimation 1328 data. Advanced ECG data processing refers to denoising, visualization and feature extraction algorithms that can be informed by the ECG electrode locations and heartbeat tags. These visualizations and extracted features may provide corroborating or orthogonal diagnostic value to a physician, as well as further insights as described throughout the disclosure.

In some embodiments of the present disclosure, the technical advantages of the disclosed the hybrid ECG-MCG system include, and are not limited to:
(i) spatial coordination of ECG electrode locations with a MCG array reference frame with higher accuracy compared to existing solutions of operator annotation with respect to patient anatomy (e.g., “pad V2 is supposed to be placed in the 4th intercostal space to the left of the sternum”);
(ii) spatial coordination of ECG cardiac activity data with cardiac source location observed by the MCG system, in the MCG array reference frame, with improved accuracy. For example, one useful source model referred to the R-wave peak's functional equivalent current dipole (ECD) location, which can be found by MCG source localization;
(iii) validation of placement of ECG electrodes, besides trusting the ECG operator);
(iv) ECG electrodes setup errors, including accidental misplacement of the limb lead electrodes, can be identified from the MCG data and corrected in the ECG data during post-processing;
(v) anatomical abnormalities such as dextrocardia (e.g., abnormal organ symmetry) can be disambiguated from ECG pad placement errors; (vi) actual patient anatomy (e.g., chest size and chest curvature) can be derived from the ECG electrode locations (presuming ECG pad placement in a known arrangement with respect to an anatomical template, such as the intercostal spaces of the ribcage). These correlative features can be used in diagnostic datasets, especially for machine learning;
(vi) Motion of the ECG electrode (e.g., due to patient motion) during the ECG+MCG scan can be used for denoising motion artifacts in the MCG data. For example,
“bad” segments of data can be ignored in the case of large motion, whereas “merely confounded” segments of data can be cleaned in the case of milder motion.
(vii) In some embodiments, a machine learning based signal processing method can be trained using data on such phenomena; and
Motion of the ECG pads (e.g., due to poor adhesion) during the ECG+MCG scan can be used for denoising artifacts in the ECG data.

### Flowcharts

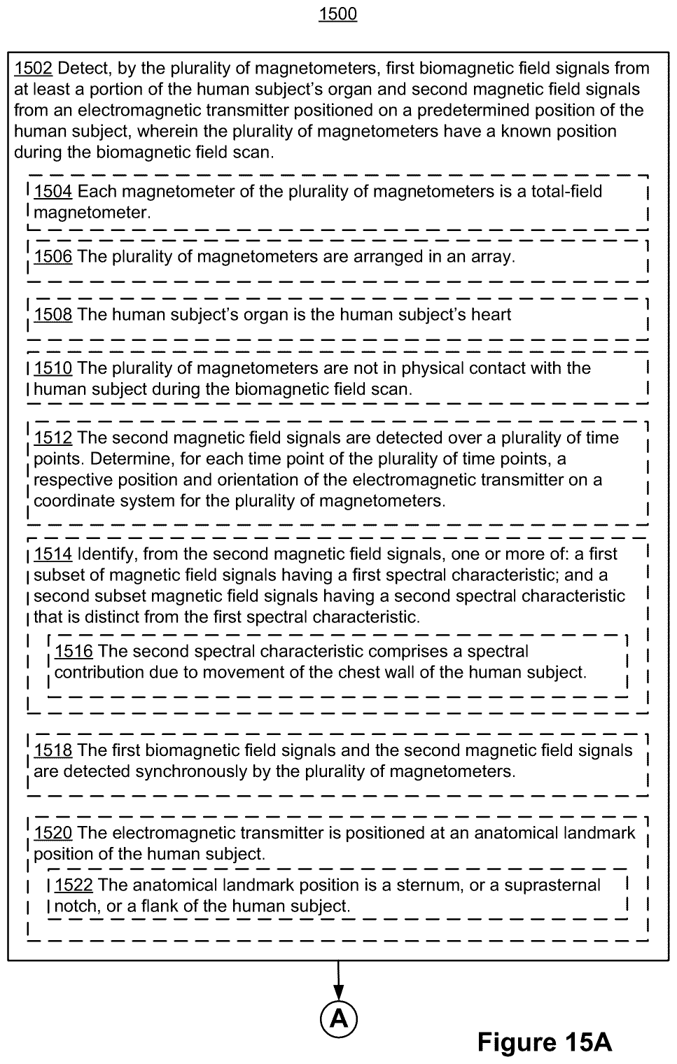

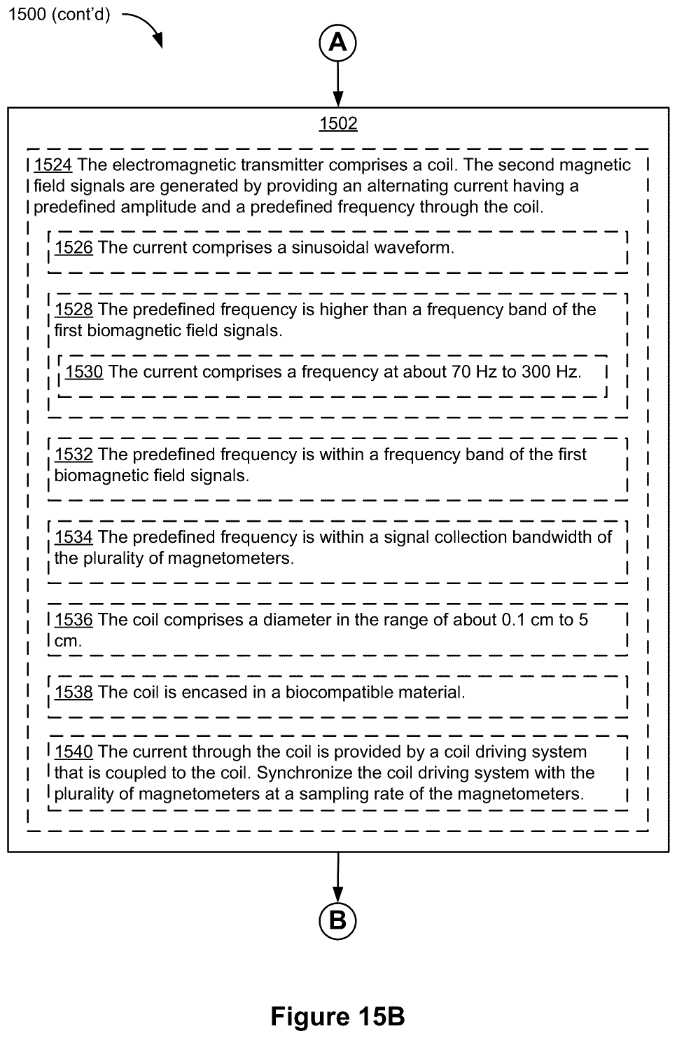

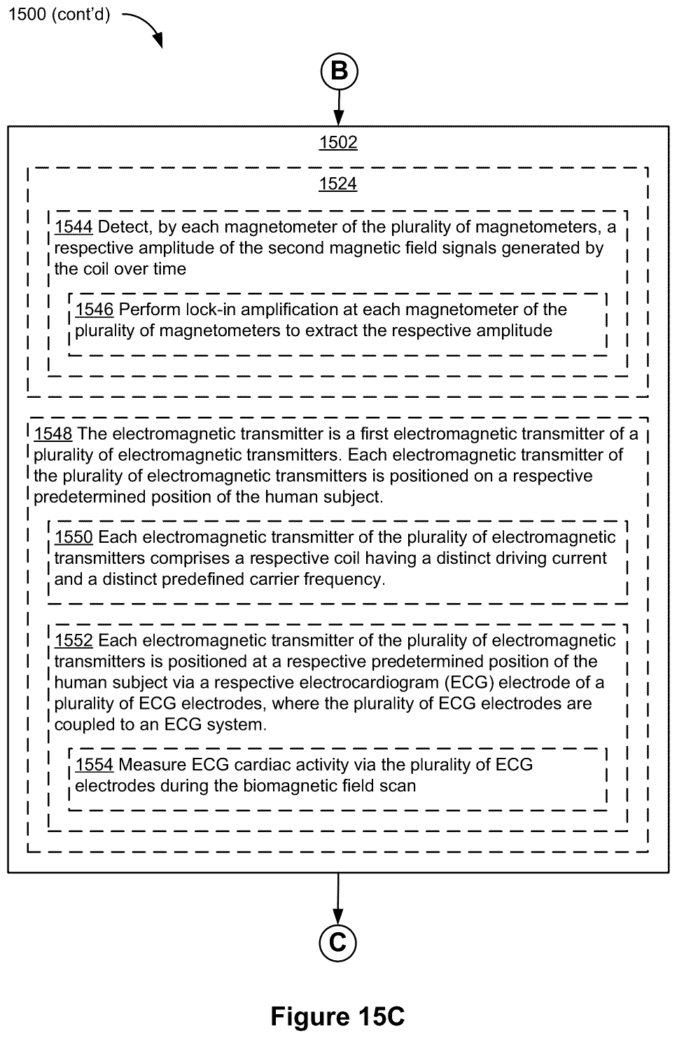

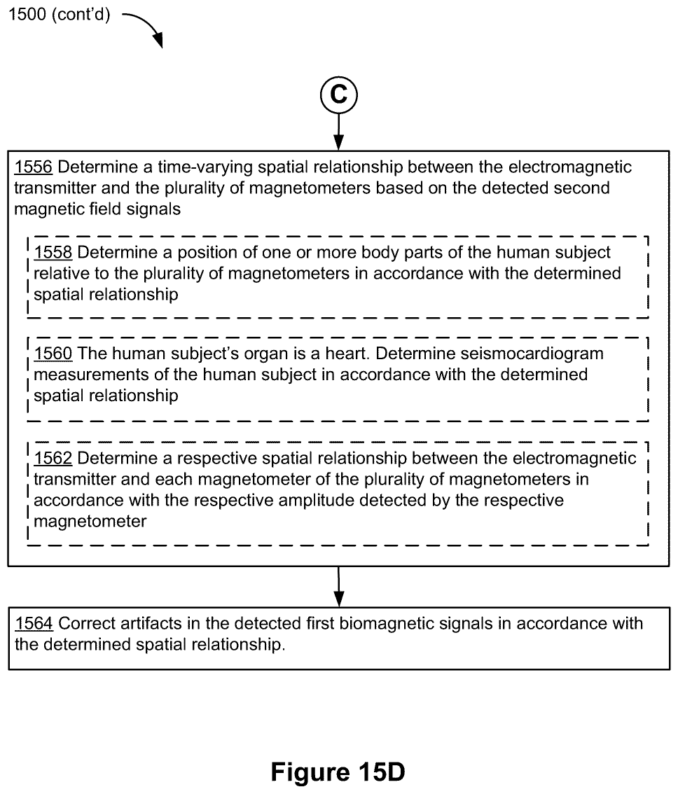

FIGS. 15A to 15D illustrate a flowchart diagram for a method 1500 of determining positional information of a human subject during a biomagnetic field scan, in accordance with some embodiments. In some embodiments, the method 1500 is performed at a computing device (e.g., computing system 120) that includes one or more processors (e.g., CPU(s) 302) and memory (e.g., memory 306). In some embodiments, the computing device is a device that is communicatively coupled to an MCG system (e.g., MCG device, or apparatus 200). In some embodiments, the computing device is a device that is integrated with an MCG system. In some embodiments, the computing device is a communicatively coupled to or integrated with a hybrid MCG-ECG system. The computing device is coupled (e.g., communicatively coupled or physically coupled) to a plurality (e.g., array) of magnetometers (e.g., magnetometers 201). Each magnetometer has a fixed, predetermined (e.g., known) position with respect to other magnetometers of the plurality of magnetometers.

In some embodiments, the operations shown in FIGS. 1A, 1B, 4, 5A, 5B, 6, 7A, 7B, 7C, 8A, 8B, 9C, 9A, 9B, 10A, 10B, 11, 12, 13A, 13B, and 14 correspond to instructions stored in the memory 306 or other non-transitory computer-readable storage medium. The computer-readable storage medium may include a magnetic or optical disk storage device, solid state storage devices such as Flash memory, or other non-volatile memory device or devices. In some embodiments, the instructions stored on the computer-readable storage medium include one or more of: source code, assembly language code, object code, or other instruction format that is interpreted by one or more processors. Some operations in the method 1500 may be combined (e.g., with method 1600, method 1700, or method 1800) and/or the order of some operations may be changed.

The method includes detecting (1502) (e.g., measuring), using the plurality of magnetometers, first biomagnetic field signals (e.g., time-varying signals, signals having one or more first amplitudes and/or first directions that change with time) from at least a portion of the human subject's organ and second magnetic field signals (e.g., time-varying signals, signals having one or more second amplitudes and/or second directions that change with time) from an electromagnetic transmitter (e.g., electromagnetic transmitter 406) positioned on a predetermined position of the human subject, where the plurality of magnetometers have a known position during the biomagnetic field scan.

In some embodiments, detecting the first biomagnetic field signals comprises continuously detecting the first biomagnetic field signals for an entire duration of the biomagnetic field scan. In some embodiments, detecting the first biomagnetic field signals comprises detecting the first biomagnetic field signals at periodic intervals (e.g., every 0.1 millisecond (10000 samples/sec), 0.5 millisecond (2000 samples/sec), 1 millisecond (1000 samples/sec), 2 millisecond (500 samples/sec), or 5 milliseconds (200 samples/sec), for an entire duration of the biomagnetic field scan.

In some embodiments, detecting the second magnetic field signals comprises continuously detecting the second magnetic field signals for an entire duration of the biomagnetic field scan. In some embodiments, detecting the second magnetic field signals comprises detecting the second magnetic field signals at periodic intervals (e.g., regular intervals, such as every 0.1 millisecond, 0.5 millisecond, 1 millisecond, 2 milliseconds, or 5 milliseconds (200 samples/sec), for an entire duration of the biomagnetic field scan. In some embodiments, the second magnetic field signals are detected at a sampling rate that provides a Nyquist frequency suitable for measuring a carrier tone of the electromagnetic transmitter.

In some embodiments, the electromagnetic transmitter has a distinct, unique magnetic signature that is different from the biomagnetic field signals.

In some embodiments, each magnetometer of the plurality of magnetometers is (1504) a total-field magnetometer.

In some embodiments, the plurality of magnetometers are (1506) arranged in an array (e.g., array 404 or on a MCG sensor panel 1212).

In some embodiments, the magnetometers have known positions relative to one another. In some embodiments, the magnetometers are positioned as a rigid array. Exemplary configurations of magnetometer array are disclosed in U.S. patent application Ser. No. 18/607,317, filed Mar. 15, 2024, titled “Systems and Methods for Biomagnetic Field Imaging,” which is incorporated by reference herein in its entirety.

In accordance with some embodiments of the present disclosure, positional information of a target organ of a human subject can be obtained when the location of each magnetometer, relative to other magnetometers, are known at each point in time during the biomagnetic field scan. For example, when the plurality of magnetometers are arranged in a rigid array, the locations of each magnetometer relative to other magnetometers in the array is always known, even if the magnetometer array moves a measurement. In the method 1500, the positional information of the target organ that is evaluated is a relative position between the magnetometer array and the human subject. In some embodiments, to discriminate between motion due to the magnetometer array and motion due to the human subject, the magnetometer array can include one or more accelerometers (e.g., sensor(s) 203) for measuring vibration and/or acceleration in the magnetometer array. Sensor data from the accelerometers can be used for correlating motion artifacts in the data.

In some embodiments, the human subject's organ is (1508) the human subject's heart.

In some embodiments, the plurality of magnetometers are not (1510) in physical contact with the human subject during the biomagnetic field scan.

In some embodiments, the second magnetic field signals are detected (1512) over a plurality of time points. The computing device determines, for each time point of the plurality of time points, a respective position and orientation (e.g., a six-dimensional coordinate, including x, y, z coordinates and their subtended angles α, β, and δ) of the electromagnetic transmitter on a coordinate system for the plurality of magnetometers.

In some embodiments, the computing device identifies (1514), from the second magnetic field signals, one or more of: a first subset of magnetic field signals having a first spectral characteristic; and a second subset magnetic field signals having a second spectral characteristic that is distinct from the first spectral characteristic.

For example, in some embodiments, the first spectral characteristic comprises lower-frequency spectral composition (e.g., components) whereas the second spectral characteristic comprises higher-frequency spectral composition (e.g., components).

In some embodiments, the first spectral characteristic includes a first amplitude (or a first range of amplitudes) and the second spectral characteristic includes a second amplitude (or a second range of amplitudes) that is higher than the first amplitude (or the first range of amplitudes).

In some embodiments, the first subset of magnetic field signals includes a first spatial distribution, and the second subset of magnetic field signals includes a second spatial distribution that is distinct from the first spatial distribution.

In some embodiments, the first subset of magnetic field signals and the second subset magnetic field signals can be separated using blind-source-separation techniques, or by regressing reference signals such as an accelerometer or chest band.

In some embodiments, the second spectral characteristic comprises (1516) a spectral contribution due to movement of the chest wall of the human subject.

In some embodiments, the first biomagnetic field signals and the second magnetic field signals are (1518) detected synchronously (e.g., made to happen at the same time, correspondence of events in time over a short period, or simultaneously) by the plurality of magnetometers.

In some embodiments, the electromagnetic transmitter is (1520) positioned at an anatomical landmark position of the human subject.

In some embodiments, the anatomical landmark position is (1522) a sternum, or a suprasternal notch (e.g., where the second rib meets the sternum), or a flank of the human subject. In some embodiments, the anatomical landmark position is any other appropriate location that can identify the anatomy of the human subject.

Referring to FIG. 15B, in some embodiments, the electromagnetic transmitter comprises (1524) a coil (e.g., a conductive coil, a coil of conductive wires). The second magnetic field signals are generated by providing an alternating current (AC current) having a predefined amplitude and a predefined frequency (e.g., carrier frequency) through the coil. The current-carrying coil produces a magnetic field that is detected by the magnetometers. When the coil moves, either due to intentional movements by the human subject (e.g., moving on the patient bed) or unintentional movements by the human subject, (e.g., movement of the chest wall, breathing, heartbeat), the movement of the coil will modulate the amplitude of the carrier frequency of the magnetometers, like an AM radio.

In some embodiments, the current comprises (1526) a sinusoidal waveform.

In some embodiments, the predefined frequency is (1528) higher than a frequency band of the first biomagnetic field signals.

In some embodiments, the current comprises (1530) a frequency at about 70 Hz to 300 Hz (e.g., within ±1%, 2%, or ±5%). In some embodiments, the current comprises a frequency at about 100 Hz and 200 Hz. In some embodiments, the frequency of the current has an upper limit that does not exceed 5000 Hz, 4000 Hz, 3000 Hz, 2000 Hz, 1000 Hz, 900 Hz, 800 Hz, 700 Hz, 600 Hz, 500 Hz, 400 Hz, 300 Hz, 200 Hz, or 100 Hz. In some embodiments, the current comprises a frequency with an upper limit that is up to any frequency within the signal bandwidth of the magnetometers used for the MCG measurements. By contrast, the first biomagnetic signals have a frequency band that is approximately 1 to 40 Hz. This frequency multiplexing approach (e.g., combining multiple signals in a single MCG channel) preserves the biomagnetic field signals while enabling integrated, synchronized, continuous measurement of the seismocardiogram.

In some embodiments, the driving frequency of the coil is higher than the frequency range of the biomagnetic field signals, and thus the first biomagnetic field signals and the second magnetic field signals do not interfere with each other (i.e., there is clean separation of the first and second magnetic field signals). In some embodiments, the driving frequency of the coil overlaps with the first biomagnetic field signals.

In some embodiments, the predefined frequency is (1532) within a frequency band of the first biomagnetic field signals.

In some embodiments, the predefined frequency is (1534) within a signal collection bandwidth of the plurality of magnetometers.

In some embodiments, the coil comprises (1536) a diameter in the range of about 0.1 cm to 5 cm.

In some embodiments, the coil is (1538) encased in a biocompatible material.

In some embodiments, the current through the coil is (1540) provided by a coil driving system (e.g., CPI driving system 408) that is coupled to the coil. The method includes synchronizing the coil driving system with the plurality of MCG sensors at a sampling rate of the MCG sensors.

With continued reference to FIG. 15C, in some embodiments, detecting, by the plurality of magnetometers, the second magnetic field signals from the electromagnetic transmitter includes detecting (1544), by each magnetometer of the plurality of magnetometers, a respective amplitude of the second magnetic field signals generated by the coil over time (e.g., during the time duration of the biomagnetic field scan).

In some embodiments, the computing device compares the respective amplitudes of the second magnetic field signals that are detected by the magnetometers and determines, in accordance with the comparing, a relative distance between the coil and a respective magnetometer, relative to other magnetometers of the plurality of magnetometers.
In some embodiments, as the coil moves (e.g., due to patient movement, or movement of the chest wall during a heartbeat or breathing), the amplitude of the carrier frequency (e.g., coil frequency) that is recorded by each of the magnetometers changes in accordance with the movement of the coil. This amplitude variation (modulation) is directly related to the distance and angle between the coil and magnetometer.

In some embodiments, detecting, by each magnetometer of the plurality of magnetometers, the respective amplitude of the second magnetic field signals includes performing (1546) lock-in amplification at each magnetometer of the plurality of magnetometers to extract the respective amplitude.

In some embodiments, the electromagnetic transmitter is (1548) a first electromagnetic transmitter of a plurality of electromagnetic transmitters. Each electromagnetic transmitter of the plurality of electromagnetic transmitters is positioned on a respective predetermined position of the human subject.

In some embodiments, each electromagnetic transmitter of the plurality of electromagnetic transmitters comprises (1550) a respective coil having a distinct (e.g., predefined) driving current (e.g., AC current with a sinusoidal tone) and a distinct (e.g., predefined) carrier frequency.

In some embodiments, each electromagnetic transmitter of the plurality of electromagnetic transmitters is (1552) positioned at a respective predetermined position of the human subject via a respective electrocardiogram (ECG) electrode (e.g., ECG pad) of a plurality of ECG electrodes. The plurality of ECG electrodes are coupled to an ECG system (e.g., ECG system 1206).

In some embodiments, the method includes measuring (1554) ECG cardiac activity via the plurality of ECG electrodes during the biomagnetic field scan.

Referring now to FIG. 15D, in some embodiments, the computing device determines (1556) a time-varying spatial relationship between the electromagnetic transmitter and the plurality of magnetometers (e.g., between the electromagnetic transmitter and each magnetometer of the plurality of magnetometers) based on the detected second magnetic field signals.

In some embodiments, the spatial relationship between the electromagnetic transmitter and the plurality of magnetometers varies with time due to movement of the coil as a result of movement of the human subject. In other words, the coil can serve as a proxy for determining/tracking the movement of the human subject during the biomagnetic field scan.

In some embodiments, the computing device determines (1558) a position of one or more body parts (e.g., head, heart, arms, legs, or torso) of the human subject relative to the plurality of magnetometers in accordance with the determined spatial relationship (e.g., by solving the magnetic inverse problem). For example, in some embodiments, the inverse problem can be solved by linear regression, L2 minimum norm estimation, beamforming, or MCMC methods. The resulting location and orientation of each coil is relative to the MCG magnetometer array).

In some embodiments, the method includes determining (1560) seismocardiogram measurements of the human subject in accordance with the determined spatial relationship. For example, seismocardiogram measurements can be determined by using spectral filtering, and time correlating these periodic signals to the heartbeat, as illustrated in FIGS. 7A and 7B.

In some embodiments, determining the spatial relationship between the electromagnetic transmitter and the plurality of magnetometers based on the detected first biomagnetic field signals and second magnetic field signals includes determining (1562) a respective spatial relationship between the electromagnetic transmitter and each magnetometer of the plurality of magnetometers in accordance with the respective amplitude detected by the respective magnetometer.

The computing device corrects (1564) artifacts (e.g., ballistocardiogram (BCG) artifacts) in the detected first biomagnetic signals in accordance with the determined spatial relationship. In some embodiments, the artifacts are corrected in a number of ways, including linear regression, gradiometry, and blind-source-separation methods.

Although FIGS. 15A to 15D illustrate a number of logical stages in a particular order, stages which are not order dependent may be reordered and other stages may be combined or broken out. Some reordering or other groupings not specifically mentioned will be apparent to those of ordinary skill in the art, so the ordering and groupings presented herein are not exhaustive. Moreover, it should be recognized that the stages could be implemented in hardware, firmware, software, or any combination thereof.

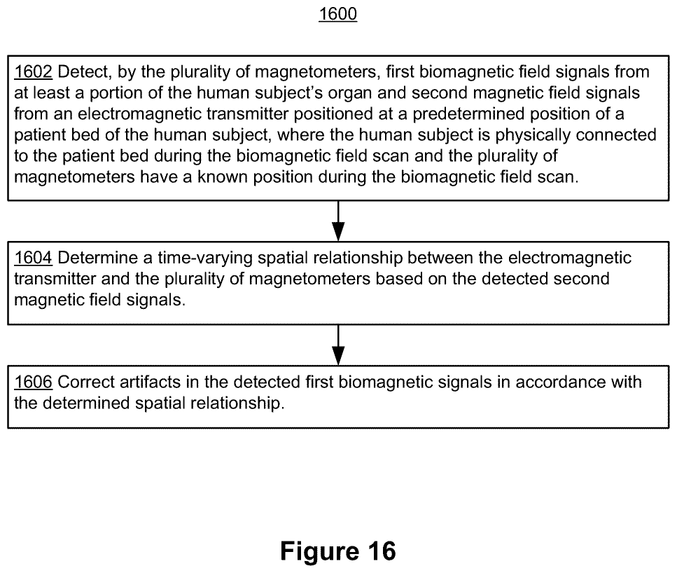

FIG. 16 illustrates a flowchart diagram for a method 1600 for determining positional information of a human subject during a biomagnetic field scan, in accordance with some embodiments. The method 1600 is performed at a computing device (e.g., computing system 120) coupled to a plurality of magnetometers (e.g., apparatus 200, magnetometers 201 or MCG system 402), one or more processors (e.g., CPU(s) 302), and memory (e.g., memory 306). In some embodiments, the computing device is a device that is communicatively coupled to an MCG system (e.g., MCG device, or apparatus 200). In some embodiments, the computing device is a device that is integrated with an MCG system. In some embodiments, the computing device is a communicatively coupled to or integrated with a hybrid MCG-ECG system. The computing device is coupled (e.g., communicatively coupled or physically coupled) to a plurality (e.g., array) of magnetometers (e.g., magnetometers 201). Additional details of the method 1600 can be found in FIGS. 1 to 15D and the accompanying descriptions, and are not repeated for the sake of brevity. Some operations in the method 1600 may be combined (e.g., with method 1500, method 1700, or method 1800) and/or the order of some operations may be changed.

The computing device detects (1602), by the plurality of magnetometers, first biomagnetic field signals from at least a portion of the human subject's organ and second magnetic field signals from an electromagnetic transmitter positioned at a predetermined position of a patient bed (e.g., exam table) of the human subject, where the human subject is physically connected to the patient bed during the biomagnetic field scan and the plurality of magnetometers have a known position during the biomagnetic field scan. In the example of FIG. 16, the electromagnetic transmitter is mounted on a patient bed instead of the patient. In some instances, one may choose to measure motion of the bed when the bed is highly magnetic, and motion transferred from the patient to the bed causes large magnetic artifacts. In some instances, it may be preferable to affix the electromagnetic transmitter on the patient bed instead of the patient when the patient cannot have anything affixed to their chest (e.g., the patient is a burn victim). While there would be loss of direct data from participant motion, large artifacts could likely still be recovered.

The computing device determines (1604) a time-varying spatial relationship between the electromagnetic transmitter and the plurality of magnetometers based on the detected second magnetic field signals.

The computing device corrects (1606) artifacts in the detected first biomagnetic signals in accordance with the determined spatial relationship.

Although FIG. 16 illustrates a number of logical stages in a particular order, stages which are not order dependent may be reordered and other stages may be combined or broken out. Some reordering or other groupings not specifically mentioned will be apparent to those of ordinary skill in the art, so the ordering and groupings presented herein are not exhaustive. Moreover, it should be recognized that the stages could be implemented in hardware, firmware, software, or any combination thereof.

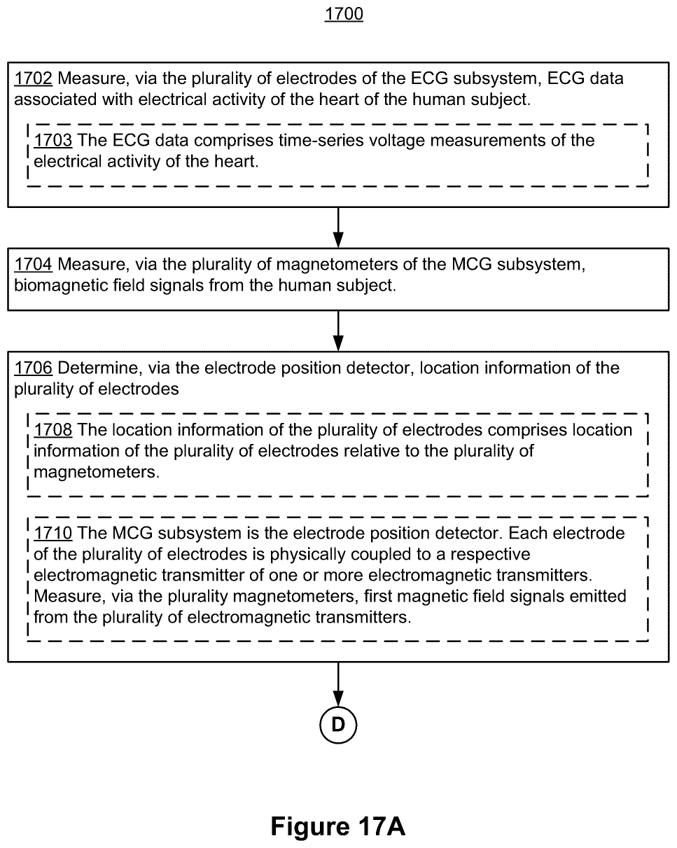

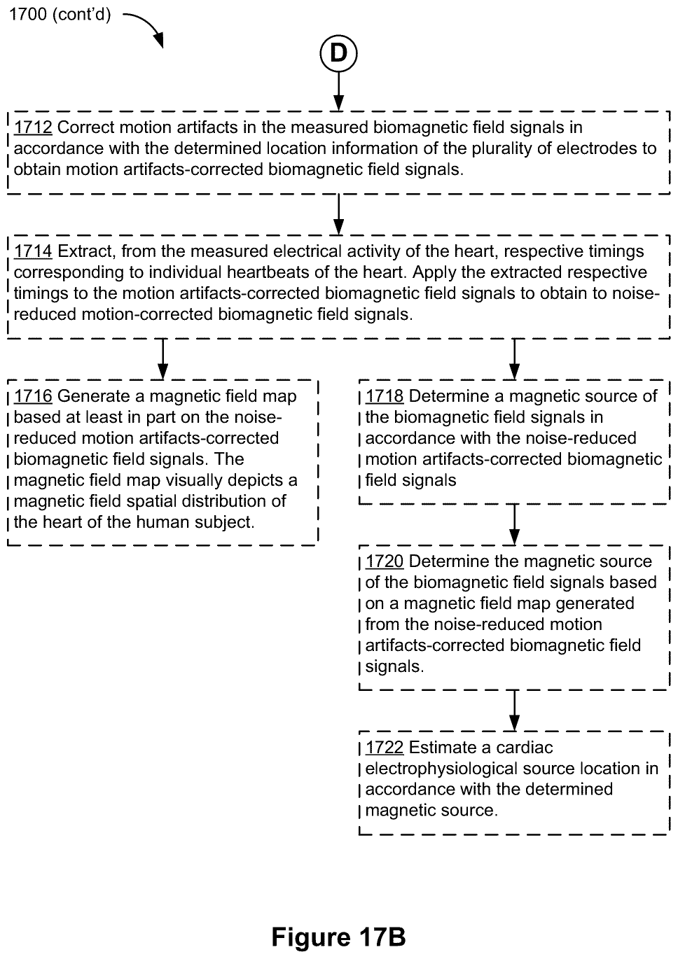

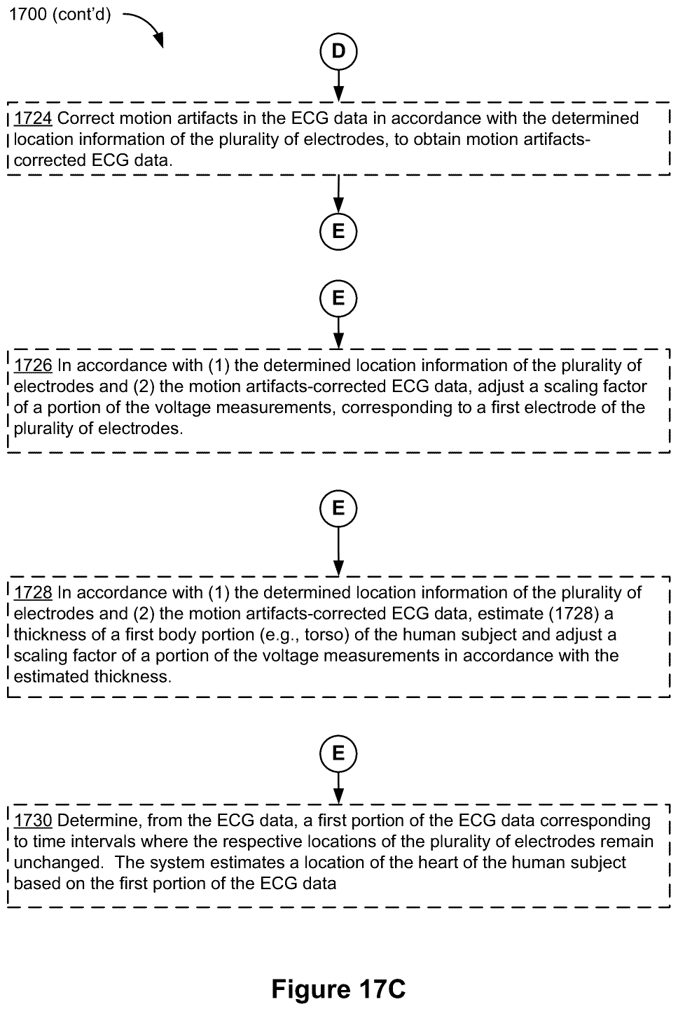

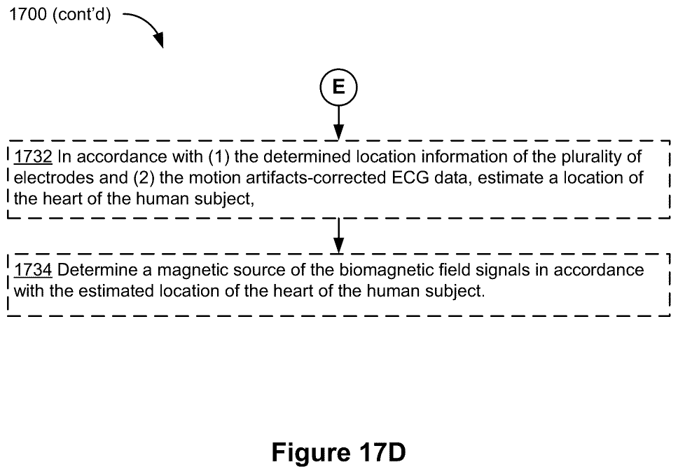

FIGS. 17A to 17D illustrate a flowchart diagram for a method 1700 of performing continuous, colocalized electrophysiology measurements on a human subject, in accordance with some embodiments. In some embodiments, the method 1700 is performed at a system (e.g., system 1200) that includes a magnetocardiography (MCG) subsystem (e.g., MCG system 402 or apparatus 200) having a plurality of magnetometers (e.g., magnetometers 201) an electrocardiography (ECG) subsystem (e.g., ECG system 1206) having a plurality of electrodes (e.g., ECG electrodes 1208) that are physically coupled to a human subject (e.g., human subject 1220 or patient 1307), and an electrode position detector (e.g., electrode position detector). In some embodiments, the plurality of magnetometers are arranged in an array (e.g., array 404) or as a MCG sensor panel (e.g., MCG sensor panel 1212). Each magnetometer has a fixed, predetermined (e.g., known) position with respect to other magnetometers of the plurality of magnetometers.

In some embodiments, the operations shown in FIGS. 1A, 1B, 4, 5A, 5B, 6, 7A, 7B, 7C, 8A, 8B, 9C, 9A, 9B, 10A, 10B, 11, 12, 13A, 13B, and 14 correspond to instructions stored in the memory of the system 1200 (e.g., memory 1301, memory 1303) or other non-transitory computer-readable storage medium. The computer-readable storage medium may include a magnetic or optical disk storage device, solid state storage devices such as Flash memory, or other non-volatile memory device or devices. In some embodiments, the instructions stored on the computer-readable storage medium include one or more of: source code, assembly language code, object code, or other instruction format that is interpreted by one or more processors. Some operations in the method 1700 may be combined and/or the order of some operations may be changed. Additional details of the method 1700 can be found in FIGS. 1 to 16 and the accompanying descriptions, and are not repeated for the sake of brevity. Some operations in the method 1700 may be combined (e.g., with method 1500, method 1600, or method 1800) and/or the order of some operations may be changed.

The system measures (1702), via the plurality of electrodes of the ECG subsystem, ECG data (e.g., ECG data 1330) associated with electrical activity of the heart of the human subject (e.g., patient 1307).

In some embodiments, the ECG data comprises (1703) time-series voltage measurements of the electrical activity of the heart.

The system measures (1704), via the plurality of magnetometers of the MCG subsystem, biomagnetic field signals (e.g., biomagnetic field signals 1306) from the human subject.

The system determines (1706), via the electrode position detector, location information of the plurality of electrodes (e.g., electrode locations 1310).

In some embodiments, the location information of the plurality of electrodes comprises (1708) location information of the plurality of electrodes relative to the plurality of magnetometers. In some embodiments, the location information of the plurality of electrodes comprises positional information in a coordinate system of the MCG subsystem. The positional information can be expressed as three-axis (e.g., (x,y,z)) coordinates or as six-axis coordinates (e.g., (X, Y, Z) corresponding to the base coordinates and the angles of rotation (RX, RY, RZ) around X, Y and Z axes of base coordinates). In some embodiments, the system determines the location information of the plurality of electrodes in a coordinate system that is common to both the ECG subsystem and MCG subsystem.

In some embodiments, the MCG subsystem is (1710) the electrode position detector. Each electrode of the plurality of electrodes is physically coupled to a respective electromagnetic transmitter of one or more electromagnetic transmitters. This is illustrated in FIG. 12. In some embodiments, determining, via the electrode position detector, the location information of the plurality of electrodes includes measuring, via the plurality magnetometers, first magnetic field signals emitted from the plurality of electromagnetic transmitters.

Referring now to FIG. 17B, in some embodiments, the system corrects (1712) motion artifacts in the measured biomagnetic field signals in accordance with the determined location information of the plurality of electrodes to obtain motion artifacts-corrected biomagnetic field signals (e.g., motion artifacts-corrected MCG data 1317).

For example, in some embodiments, the location information (e.g., positional information) of the ECG electrodes can be used as regressors in the MCG data because the motion of the electrodes is expected to correlate directly to magnetic artifacts in the signal, up to a linear scale factor. Therefore, the time series positional information can be linearly regressed from the MCG data. In some embodiments, the regression coefficient between these two time-series datasets (e.g., time-series data of measured biomagnetic field signals and time-series data of electrode locations) can be determined via mean scaling, median scaling, least-squares, PCA, or regression algorithms.

In some embodiments, the system extracts (1714), from the measured electrical activity of the heart, respective timings corresponding to individual heartbeats of the heart (e.g., using the peak of the R-wave). The respective timings corresponding to individual heartbeats of the heart are also referred to as heartbeat tags (e.g., heartbeat tags 1312) in this disclosure. The system applies the extracted respective timings to the motion artifacts-corrected biomagnetic field signals to obtain noise-reduced motion-corrected biomagnetic field signals (e.g., noise-reduced, motion-corrected MCG data 1319). In some embodiments, the noise-reduced motion-corrected biomagnetic field signals have improved signal-to-noise ratio compared to the motion-corrected biomagnetic field signals.

FIG. 17B, as well as FIGS. 13A and 13B, illustrate that the noise-reduced, motion-corrected MCG data 1319 can be utilized in several ways. As one example, in some embodiments, the system generates (1716) a magnetic field map based at least in part on the noise-reduced motion artifacts-corrected biomagnetic field signals, where the magnetic field map visually depicts a magnetic field spatial distribution of the heart of the human subject. As another example, in some embodiments, the system determines (1718) a magnetic source of the biomagnetic field signals in accordance with the noise-reduced motion artifacts-corrected biomagnetic field signals.

In some embodiments, the system determines (1720) the magnetic source of the biomagnetic field signals based on a magnetic field map generated from the noise-reduced motion artifacts-corrected biomagnetic field signals. In some embodiments, the system estimates (1722) (e.g., determines) a cardiac electrophysiological source location in accordance with the determined magnetic source. For example, the system can estimate the current density distribution of the heart, using techniques such as electric current dipole reductions and/or other.

With continued reference to FIG. 17C, and also discussed with respect to FIGS. 13A and 13B, the quality and integrity of ECG data can be influenced by motion artifacts due to movement of the ECG electrodes due as a result of patient movement, sliding of the ECG electrode pads, and/or movement of the patient chest wall due to heartbeats or breathing. In some embodiments, the hybrid system utilizes location information of the ECG electrodes (that is determined by the electrode position detector 1210 and/or the MCG system 402) and corrects (1724) motion artifacts in the ECG data in accordance with the determined location information of the plurality of electrodes, to obtain motion artifacts-corrected ECG data (e.g., motion artifacts-corrected ECG data 1333).

In some embodiments, the system, in accordance with (1) the determined location information of the plurality of electrodes and (2) the motion artifacts-corrected ECG data, performs location-aware signal processing (e.g., location-aware signal processing 1334). For example, in some embodiments, the ECG data comprises time-series voltage measurements of the electrical activity of the heart. The system adjusts (1726) a scaling factor of a portion of the voltage measurements, corresponding to a first ECG electrode (e.g., electrode pad) of the plurality of ECG electrodes.

In some embodiments, in accordance with (1) the determined location information of the plurality of electrodes and (2) the motion artifacts-corrected ECG data, the system estimates (1728) a thickness of a first body portion (e.g., torso) of the human subject and adjusts a scaling factor of a portion of the voltage measurements in accordance with the estimated thickness.

In some embodiments, the system determines (1730), from the ECG data, a first portion of the ECG data corresponding to time intervals where the respective locations of the plurality of electrodes remain unchanged. The system estimates a location of the heart of the human subject based on the first portion of the ECG data.

Referring now to FIG. 17D, in some embodiments, the system, in accordance with (1) the determined location information of the plurality of electrodes and (2) the motion artifacts-corrected ECG data, estimates (1732) a location of the heart of the human subject. In some embodiments, the system determines (1734) a magnetic source of the biomagnetic field signals in accordance with the estimated location of the heart of the human subject.

Although FIGS. 17A to 17D illustrate a number of logical stages in a particular order, stages which are not order dependent may be reordered and other stages may be combined or broken out. Some reordering or other groupings not specifically mentioned will be apparent to those of ordinary skill in the art, so the ordering and groupings presented herein are not exhaustive. Moreover, it should be recognized that the stages could be implemented in hardware, firmware, software, or any combination thereof.

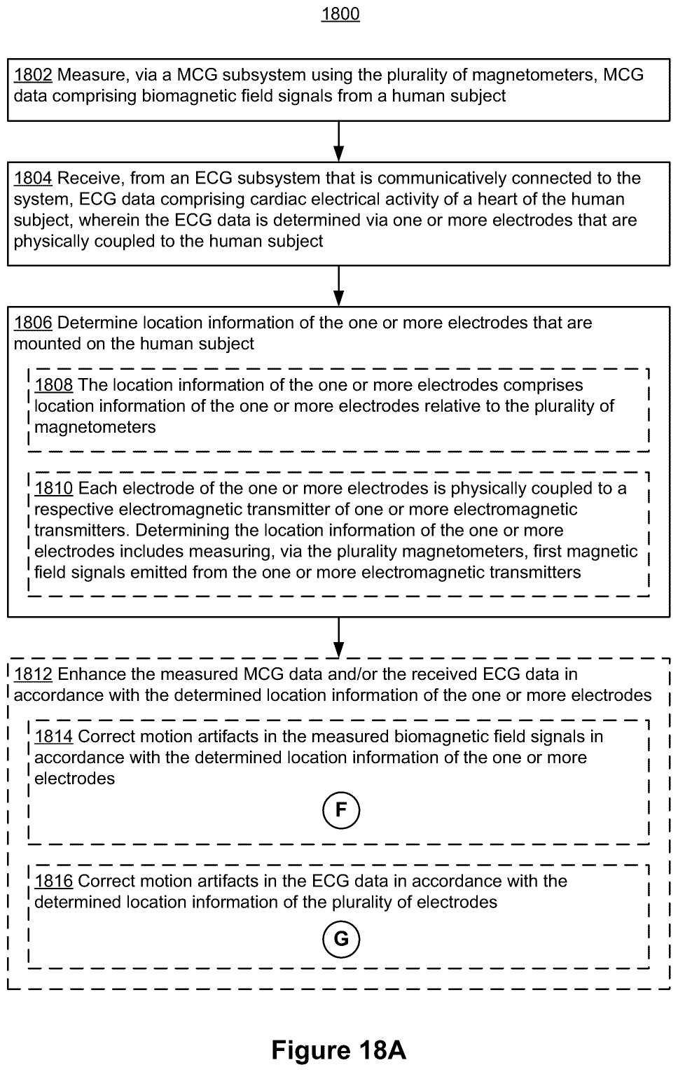

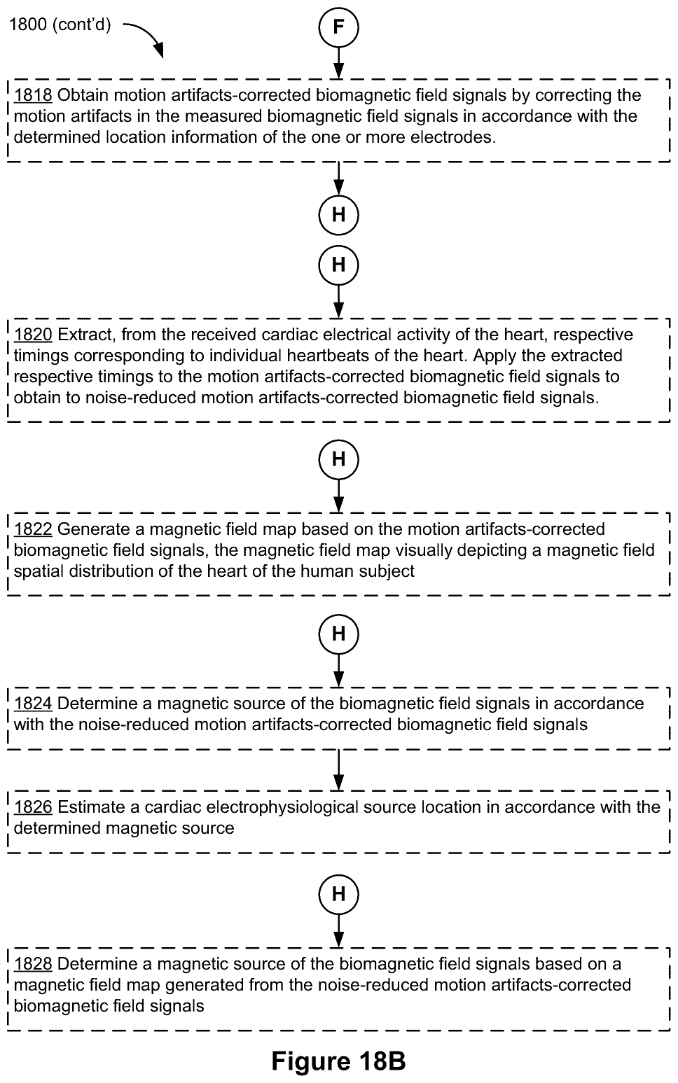

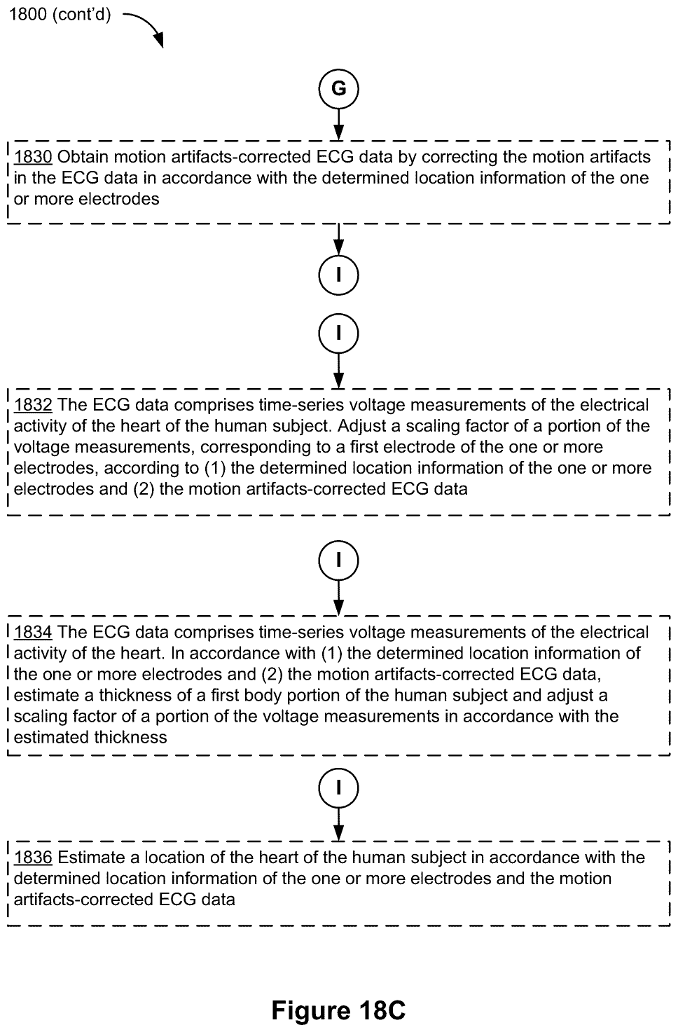

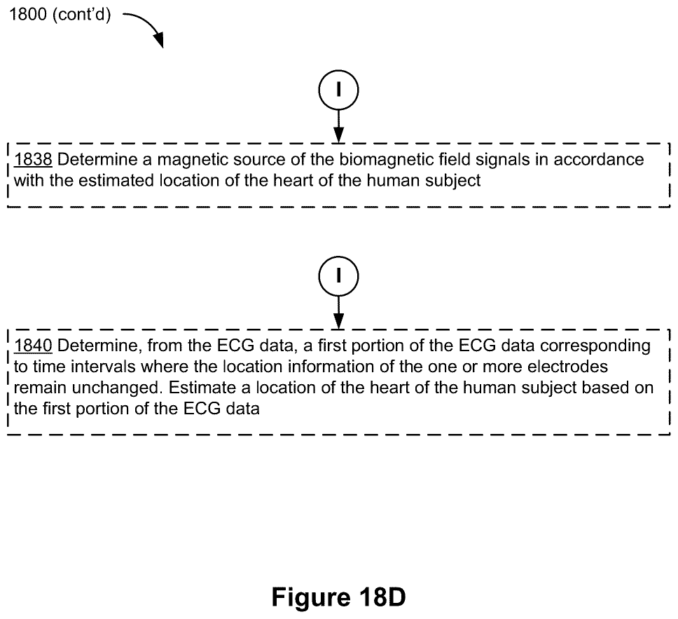

FIGS. 18A to 18D illustrate a flowchart diagram for a method 1800 of performing continuous, colocalized electrophysiology measurements on a human subject, in accordance with some embodiments. In some embodiments, the method 1800 is performed at a system (e.g., MCG system 402, apparatus 200, computer system 120, or system 1200) that includes one or more processors (e.g., CPU(s) 202, CPU(s) 302, processing circuitry 1302), memory (e.g., memory 206, memory 306, memory 1301), and a magnetocardiography (MCG) subsystem (e.g., MCG system 402 having a plurality of magnetometers (e.g., MCG magnetometers array 1305, magnetometers 201). In some embodiments, the plurality of magnetometers are arranged in an array (e.g., array 404) or as a MCG sensor panel (e.g., MCG sensor panel 1212). Each magnetometer has a fixed, predetermined (e.g., known) position with respect to other magnetometers of the plurality of magnetometers. In some embodiments, the operations shown in FIGS. 1A, 1B, 4, 5A, 5B, 6, 7A, 7B, 7C, 8A, 8B, 9C, 9A, 9B, 10A, 10B, 11, 12, 13A, 13B, and 14 correspond to instructions stored in the memory of the system or other non-transitory computer-readable storage medium. The computer-readable storage medium may include a magnetic or optical disk storage device, solid state storage devices such as Flash memory, or other non-volatile memory device or devices. In some embodiments, the instructions stored on the computer-readable storage medium include one or more of: source code, assembly language code, object code, or other instruction format that is interpreted by one or more processors. Some operations in the method 1800 may be combined and/or the order of some operations may be changed. Additional details of the method 1800 can be found in FIGS. 1 to 17 and the accompanying descriptions, and are not repeated for the sake of brevity. Some operations in the method 1800 may be combined (e.g., with method 1500, method 1600, or method 1700) and/or the order of some operations may be changed.

The system measures (1802), via the MCG subsystem using the plurality of magnetometers, MCG data comprising biomagnetic field signals from a human subject.

The system receives (1804), from one or more ECG electrodes that are physically coupled to the human subject, ECG data comprising cardiac electrical activity of a heart of the human subject. In some embodiments, the one or more ECG electrodes are physically coupled to an ECG system (e.g., ECG system 1206) that is communicatively connected to the system. In some embodiments, the system is a hybrid (or combined) MCG/ECG system, and the ECG system (e.g., ECG subsystem) is part of the hybrid system that also includes the MCG subsystem. In some embodiments, the ECG system is separate from the system that includes the MCG subsystem. For example, the ECG system may be a standalone system that is located in the same room as the MCG subsystem.

The system determines (1806) location information of the one or more electrodes that are mounted on the human subject.

In some embodiments, the location information of the one or more electrodes comprises (1808) location information of the one or more electrodes relative to the plurality of magnetometers.

In some embodiments, each electrode of the one or more electrodes is (1810) physically coupled to a respective electromagnetic transmitter of one or more electromagnetic transmitters. Determining the location information of the one or more electrodes includes measuring, via the plurality of magnetometers, first magnetic field signals emitted from the one or more electromagnetic transmitters.

In some embodiments, the system enhances (1812) the measured MCG data and/or the received ECG data in accordance with the determined location information of the one or more electrodes. For example, in some embodiments, the system corrects (1814) motion artifacts in the measured biomagnetic field signals in accordance with the determined location information of the one or more electrodes, to obtain motion artifacts-corrected biomagnetic field signals (e.g., motion artifacts-corrected MCG data 1317). In another example, in some embodiments, the system corrects (1816) motion artifacts in the ECG data in accordance with the determined location information of the one or more electrodes, to obtain motion artifacts-corrected ECG data (motion artifacts-corrected ECG data 1333).

Referring to FIG. 18B, in some embodiments, the system obtains (1818) motion artifacts-corrected biomagnetic field signals (e.g., motion artifacts-corrected MCG data 1317) by correcting the motion artifacts in the measured biomagnetic field signals in accordance with the determined location information of the one or more electrodes.

In some embodiments, the system extracts (1820), from the received cardiac electrical activity of the heart, respective timings corresponding to individual heartbeats of the heart (e.g., heartbeat tags 1312) and applies the extracted respective timings to the motion artifacts-corrected biomagnetic field signals to obtain to noise-reduced motion artifacts-corrected biomagnetic field signals (e.g., noise-reduced motion-corrected MCG data 1319).

In some embodiments, the system generates (1822) a magnetic field map (e.g., a magnetic field map 1324) based on the motion artifacts-corrected biomagnetic field signals, the magnetic field map visually depicting a magnetic field spatial distribution of the heart of the human subject.

In some embodiments, the system determines (1824) a magnetic source of the biomagnetic field signals (e.g., via source reconstruction 1320) in accordance with the noise-reduced motion artifacts-corrected biomagnetic field signals.

In some embodiments, the system estimates (1826) a cardiac electrophysiological source location (e.g., cardiac electrophysiological source location 1328) in accordance with the determined magnetic source.

In some embodiments, the system determines (1828) a magnetic source of the biomagnetic field signals based on a magnetic field map (e.g., magnetic field map 1324) generated from the noise-reduced motion artifacts-corrected biomagnetic field signals.

Referring now to FIG. 18C, in some embodiments, the system obtains (1830) motion artifacts-corrected ECG data (e.g., motion artifacts-corrected ECG data 1333) by correcting the motion artifacts in the ECG data in accordance with the determined location information of the one or more electrodes.

In some embodiments, the ECG data comprises (1832) time-series voltage measurements of the electrical activity of the heart of the human subject. The system adjusts a scaling factor of a portion of the voltage measurements, corresponding to a first electrode of the one or more electrodes, according to (1) the determined location information of the one or more electrodes and (2) the motion artifacts-corrected ECG data.

In some embodiments, the ECG data comprises (1834) time-series voltage measurements of the electrical activity of the heart. The system, in accordance with (1) the determined location information of the one or more electrodes and (2) the motion artifacts-corrected ECG data, estimates a thickness of a first body portion of the human subject and adjusts a scaling factor of a portion of the voltage measurements in accordance with the estimated thickness.

In some embodiments, the system estimates (1836) a location of the heart of the human subject in accordance with the determined location information of the one or more electrodes and the motion artifacts-corrected ECG data.

With continued reference to FIG. 18D, in some embodiments, the system determines (1838) a magnetic source of the biomagnetic field signals in accordance with the estimated location of the heart of the human subject.

In some embodiments, the system determines (1840), from the ECG data, a first portion of the ECG data corresponding to time intervals where the location information of the one or more electrodes remain unchanged, and estimates a location of the heart of the human subject based on the first portion of the ECG data.

Although FIGS. 18A to 18D illustrate a number of logical stages in a particular order, stages which are not order dependent may be reordered and other stages may be combined or broken out. Some reordering or other groupings not specifically mentioned will be apparent to those of ordinary skill in the art, so the ordering and groupings presented herein are not exhaustive. Moreover, it should be recognized that the stages could be implemented in hardware, firmware, software, or any combination thereof.

Turning now to some example embodiments:

(A1) In accordance with some embodiments, a method for determining positional information of a human subject during a biomagnetic field scan is performed at a computing device coupled to a plurality of magnetometers, one or more processors, and memory. The method includes (1) detecting, by the plurality of magnetometers: (a) first biomagnetic field signals from at least a portion of an organ of the human subject; and (b) second magnetic field signals from an electromagnetic transmitter positioned on a predetermined position of the human subject, wherein the plurality of magnetometers have a known position during the biomagnetic field scan; (2) determining a time-varying spatial relationship between the electromagnetic transmitter and the plurality of magnetometers based on the detected second magnetic field signals; and (3) correcting artifacts in the detected first biomagnetic signals in accordance with the determined spatial relationship.

(A2) In some embodiments of A1, the second magnetic field signals are detected over a plurality of time points; and the method includes determining, for each time point of the plurality of time points, a respective position and orientation of the electromagnetic transmitter on a coordinate system for the plurality of magnetometers.

(A3) In some embodiments of A1 or A2, the method includes determining a position of one or more body parts of the human subject relative to the plurality of magnetometers in accordance with the determined spatial relationship.

(A4) In some embodiments of any of A1-A3, the method includes identifying, from the second magnetic field signals, one or more of: (i) a first subset of magnetic field signals having a first spectral characteristic and (ii) a second subset magnetic field signals having a second spectral characteristic that is distinct from the first spectral characteristic.

(A5) In some embodiments of A4, the second spectral characteristic comprises a spectral contribution due to movement of a chest wall of the human subject.

(A6) In some embodiments of any of A1-A5, the organ of the human subject is a heart; and the method further includes determining seismocardiogram measurements of the human subject in accordance with the determined spatial relationship.

(A7) In some embodiments of any of A1-A6, the first biomagnetic field signals and the second magnetic field signals are detected synchronously by the plurality of magnetometers.

(A8) In some embodiments of any of A1-A7, the electromagnetic transmitter is positioned at an anatomical landmark position of the human subject.

(A9) In some embodiments of A8, the anatomical landmark position is a sternum, or a suprasternal notch, or a flank of the human subject.

(A10) In some embodiments of any of A1-A9, the electromagnetic transmitter comprises a coil; and the second magnetic field signals are generated by providing an alternating current having a predefined amplitude and a predefined frequency through the coil.

(A11) In some embodiments of A10, the alternating current comprises a sinusoidal waveform.

(A12) In some embodiments of A10 or A11, the predefined frequency of the alternating current is higher than a frequency band of the first biomagnetic field signals.

(A13) In some embodiments of A10 or A11, the predefined frequency of the alternating current is within a frequency band of the first biomagnetic field signals.

(A14) In some embodiments of any of A10-A13, the predefined frequency of the alternating current is within a signal collection bandwidth of the plurality of magnetometers.

(A15) In some embodiments of any of A10-A14, a diameter of the coil is about 0.1 cm to 5 cm.

(A16) In some embodiments of any of A10-A15, the coil is encased in a biocompatible material.

(A17) In some embodiments of any of A10-A16, the alternating current comprises a frequency of about 70 Hz to 300 Hz.

(A18) In some embodiments of any of A10-A17, the alternating current through the coil is provided by a coil driving system that is coupled to the coil, and the method includes synchronizing the coil driving system with the plurality of magnetometers at a sampling rate of the magnetometers.

(A19) In some embodiments of any of A10-A18, wherein detecting, by the plurality of magnetometers, the second magnetic field signals from the electromagnetic transmitter includes: detecting, by each magnetometer of the plurality of magnetometers, a respective amplitude of the second magnetic field signals generated by the coil over time.

(A20) In some embodiments of A19, determining the spatial relationship between the electromagnetic transmitter and the plurality of magnetometers based on the detected first biomagnetic field signals and second magnetic field signals includes: determining a respective spatial relationship between the electromagnetic transmitter and each magnetometer of the plurality of magnetometers in accordance with the respective amplitude detected by the respective magnetometer.

(A21) In some embodiments of A19 or A20, detecting, by each magnetometer of the plurality of magnetometers, the respective amplitude of the second magnetic field signals includes: performing lock-in amplification at each magnetometer of the plurality of magnetometers to extract the respective amplitude.

(A22) In some embodiments of any of A1-A21, the electromagnetic transmitter is a first electromagnetic transmitter of a plurality of electromagnetic transmitters; and each electromagnetic transmitter of the plurality of electromagnetic transmitters is positioned on a respective predetermined position of the human subject.

(A23) In some embodiments of A22, each electromagnetic transmitter of the plurality of electromagnetic transmitters comprises a respective coil having a distinct driving current and a distinct predefined frequency.

(A24) In some embodiments of A22 or A23, each electromagnetic transmitter of the plurality of electromagnetic transmitters is positioned at a respective predetermined position of the human subject via a respective electrocardiogram (ECG) electrode of a plurality of ECG electrodes, wherein the plurality of ECG electrodes are coupled to an ECG system.

(A25) In some embodiments of A24, the method further includes measuring ECG cardiac activity via the plurality of ECG electrodes during the biomagnetic field scan.

(A26) In some embodiments of any of A1-A25, each magnetometer of the plurality of magnetometers is a total-field magnetometer.

(A27) In some embodiments of any of A1-A26, the plurality of magnetometers are arranged in an array.

(A28) In some embodiments of any of A1-A27, the organ of the human subject is a heart.

(A29) In some embodiments of any of A1-A28, the plurality of magnetometers are not in physical contact with the human subject during the biomagnetic field scan.

(B1) In accordance with some embodiments, a method for determining positional information of a target organ of a human subject during a biomagnetic field scan is performed at a computing device coupled to a plurality of magnetometers, one or more processors, and memory. The method includes (1) detecting, by the plurality of magnetometers: (a) first biomagnetic field signals from at least a portion of the human subject's organ; and (b) second magnetic field signals from an electromagnetic transmitter positioned at a predetermined position of a patient bed of the human subject, wherein the human subject is physically connected to the patient bed during the biomagnetic field scan and the plurality of magnetometers have a known position during the biomagnetic field scan; (2) determining a time-varying spatial relationship between the electromagnetic transmitter and the plurality of magnetometers based on the detected second magnetic field signals; and (3) correcting artifacts in the detected first biomagnetic signals in accordance with the determined spatial relationship.

(C1) In accordance with some embodiments, a system coupled to a plurality of magnetometers comprises one or more processors, memory, and one or more programs stored in the memory for execution by the one or more processors, the one or more programs comprising instructions for performing the method of any of A1-A29 and B1.

(D1) In accordance with some embodiments, a computer readable storage medium storing computer-executable instructions that, when executed by one or more processors of a system that is coupled to a plurality of magnetometers, cause the system to perform the method of any of A1-A29 and B1.

(E1) In accordance with some embodiments, a method is performed at a system that includes one or more processors, memory, and a magnetocardiography (MCG) subsystem having a plurality of magnetometers. The method includes (1) measuring, via the MCG subsystem using the plurality of magnetometers, MCG data comprising biomagnetic field signals from a human subject; (2) receiving, from one or more ECG electrodes that are physically coupled to the human subject, ECG data comprising cardiac electrical activity of a heart of the human subject; and (3) determining location information of the one or more electrodes that are mounted on the human subject.

(E2) In some embodiments of E1, the location information of the one or more electrodes comprises location information of the one or more electrodes relative to the plurality of magnetometers.

(E3) In some embodiments of E1 or E2, each electrode of the one or more electrodes is physically coupled to a respective electromagnetic transmitter of one or more electromagnetic transmitters; and determining the location information of the one or more electrodes includes measuring, via the plurality of magnetometers, first magnetic field signals emitted from the one or more electromagnetic transmitters.

(E4) In some embodiments of any of E1-E3, the method further includes correcting motion artifacts in the measured biomagnetic field signals in accordance with the determined location information of the one or more electrodes, to obtain motion artifacts-corrected biomagnetic field signals.

(E5) In some embodiments of E4, the method further includes (i) extracting, from the received cardiac electrical activity of the heart, respective timings corresponding to individual heartbeats of the heart; and (ii) applying the extracted respective timings to the motion artifacts-corrected biomagnetic field signals to obtain to noise-reduced motion artifacts-corrected biomagnetic field signals.

(E6) In some embodiments of E4 or E5, the method further includes generating a magnetic field map based on the motion artifacts-corrected biomagnetic field signals, the magnetic field map visually depicting a magnetic field spatial distribution of the heart of the human subject.

(E7) In some embodiments of any of E4-E6, the method further includes determining a magnetic source of the biomagnetic field signals in accordance with the noise-reduced motion artifacts-corrected biomagnetic field signals.

(E8) In some embodiments of E7, the method further includes estimating a cardiac electrophysiological source location in accordance with the determined magnetic source.

(E9) In some embodiments of any of E4-E8, the method further includes determining a magnetic source of the biomagnetic field signals based on a magnetic field map generated from the noise-reduced motion artifacts-corrected biomagnetic field signals.

(E10) In some embodiments of any of E1-E9, the method further includes correcting motion artifacts in the ECG data in accordance with the determined location information of the one or more electrodes, to obtain motion artifacts-corrected ECG data.

(E11) In some embodiments of E10, the ECG data comprises time-series voltage measurements of the electrical activity of the heart of the human subject; and the method further includes adjusting a scaling factor of a portion of the voltage measurements, corresponding to a first electrode of the one or more electrodes, according to (1) the determined location information of the one or more electrodes and (2) the motion artifacts-corrected ECG data.

(E12) In some embodiments of E10 or E11, the ECG data comprises time-series voltage measurements of the electrical activity of the heart; and the method further includes: in accordance with (1) the determined location information of the one or more electrodes and (2) the motion artifacts-corrected ECG data: estimating a thickness of a first body portion of the human subject; and adjusting a scaling factor of a portion of the voltage measurements in accordance with the estimated thickness.

(E13) In some embodiments of any of E10-E12, the method further includes estimating a location of the heart of the human subject in accordance with the determined location information of the one or more electrodes and the motion artifacts-corrected ECG data.

(E14) In some embodiments of E13, the method further includes determining a magnetic source of the biomagnetic field signals in accordance with the estimated location of the heart of the human subject.

(E15) In some embodiments of E14, the method further includes determining, from the ECG data, a first portion of the ECG data corresponding to time intervals where the location information of the one or more electrodes remain unchanged; and estimating a location of the heart of the human subject based on the first portion of the ECG data.

(F1) In accordance with some embodiments, a system comprises a magnetocardiography (MCG) subsystem having a plurality of magnetometers, one or more processors, memory, and one or more programs stored in the memory for execution by the one or more processors, the one or more programs comprising instructions for performing the method of any of E1-E15

(G1) In accordance with some embodiments, a computer readable storage medium stores computer-executable instructions that, when executed by one or more processors of a system that includes a magnetocardiography (MCG) subsystem having a plurality of magnetometers, cause the system to perform the method of any of E1-E15.

(H1) In accordance with some embodiments, a system comprises a magnetocardiography (MCG) subsystem including a plurality of magnetometers, one or more processors, memory, and one or more programs stored in the memory for execution by the one or more processors. The one or more programs include instructions for: (1) measuring, via the MCG subsystem using the plurality of magnetometers, MCG data comprising biomagnetic field signals from a living subject; (2) receiving, from one or more ECG electrodes that are physically coupled to the living subject, ECG data comprising cardiac electrical activity of the living subject; (3) determining location information of the one or more electrodes that are mounted on the living subject; and (4) enhancing the measured MCG data and/or the received ECG data according to determined location information of the one or more electrodes.

(H2) In some embodiments of H1, each electrode of the one or more electrodes is physically coupled to a respective electromagnetic transmitter of one or more electromagnetic transmitters; and the instructions for determining the location information of the one or more electrodes that are mounted on the living subject include instructions for determining the location information of the one or more electrodes by measuring, via the plurality of magnetometers, first magnetic field signals emitted from the one or more electromagnetic transmitters.

(H3) In some embodiments of H1 or H2, the system further includes one or more cameras. The instructions for determining the location information of the one or more electrodes that are mounted on the living subject include instructions for acquiring imaging data of the one or more electrodes using the one or more cameras.

(H4) In some embodiments of any of H1-H3, each electrode of the one or more electrodes comprises a respective acoustic beacon of one or more acoustic beacons; and the instructions for determining the location information of the one or more electrodes that are mounted on the living subject include instructions for determining the location information of the one or more electrodes by measuring acoustic signals emitted from the one or more acoustic beacons.

(I1) In accordance with some embodiments, a system, comprises (i) a magnetocardiography (MCG) subsystem including a plurality of magnetometers; (ii) an electrocardiography (ECG) subsystem including one or more electrodes; and (iii) an electrode position detector. During use of the system, (1) the MCG subsystem is configured to measure MCG data comprising biomagnetic field signals from a living subject via the plurality of magnetometers; (2) the electrode position detector is configured to determine location information of the one or more electrodes that are mounted on the living subject; (3) the ECG subsystem is configured to measure ECG data comprising cardiac electrical activity of a heart of the living subject via the one or more electrodes; and (4) the system is configured to enhance the measured MCG data and/or the measured ECG data in accordance with the determined location information of the one or more electrodes.

(I2) In some embodiments of I1, the system is configured to enhance the measured MCG data in accordance with the measured MCG data and the determined location information of the one or more electrodes.

(I3) In some embodiments of I1 or 12, the system is configured to enhance the measured ECG data in accordance with the measured MCG data and the determined location information of the one or more electrodes.

(I4) In some embodiments of any of I1-I3, the system is configured to perform an overall health assessment of the living subject based on integration of the measured MCG data and the measured ECG data.

(I5) In some embodiments of any of I1-I4, each electrode of the one or more electrodes is physically coupled to a respective electromagnetic transmitter of one or more electromagnetic transmitters. The MCG subsystem is the electrode position detector and is configured to determine the location information of the one or more electrodes by measuring, via the plurality of magnetometers, first magnetic field signals emitted from the one or more electromagnetic transmitters.

(I6) In some embodiments of I5, the MCG subsystem is further configured to determine a spatial relationship between the plurality of magnetometers and the one or more electromagnetic transmitters based on the measured first magnetic field signals.

(I7) In some embodiments of I5 or 16, the first magnetic field signals are measured over a plurality of time points; and the MCG subsystem is further configured to determine the location information of the one or more electrodes by tracking respective locations of one or more electrodes over the plurality of time points.

(I8) In some embodiments of I7, the MCG subsystem is further configured to determine, for each time point of the plurality of time points, a respective position and orientation of the one or more electromagnetic transmitters on a coordinate system for the plurality of magnetometers.

(I9) In some embodiments of any of I5-I8, each electromagnetic transmitter of the one or more electromagnetic transmitters is configured to emit a unique electromagnetic frequency.

(I10) In some embodiments of any of I5-I9, the MCG subsystem is configured to measure the biomagnetic field signals concurrently with the first magnetic signals.

(I11) In some embodiments of any of I5-I10, the MCG subsystem is further configured to measure the first magnetic field signals asynchronously from the biomagnetic field signals.

(I12) In some embodiments of any of I5-I11, the one or more electrodes comprises a plurality of electrodes that are each physically coupled to a respective electromagnetic transmitter of a plurality of electromagnetic transmitters, and each of the electromagnetic transmitters is configured to emit a unique electromagnetic frequency; and the MCG subsystem is further configured to measure the first magnetic field signals synchronously with the biomagnetic field signals.

(I13) In some embodiments of I12, the plurality of electromagnetic transmitters have emission frequencies that are distinct from a collection bandwidth of the biomagnetic field signals.

(I14) In some embodiments of any of I5-I13, each electromagnetic transmitter of the one or more electromagnetic transmitters comprises a respective coil of one or more coils; and the MCG system includes a coil-driving component that is configured to provide, to the respective coil, an alternating current having a respective predefined amplitude and a respective predefined frequency.

(I15) In some embodiments of I14, wherein the coil-driving component is further configured to provide, to each coil of the one or more coils, an alternating current having a respective distinct amplitude and a respective distinct frequency.

(I16) In some embodiments of I14 or 115, the predefined frequency is within a bandwidth of the plurality of magnetometers.

(I17) In some embodiments of any of I14-I16, the respective coil has a diameter of about 0.1 cm to 5 cm.

(I18) In some embodiments of any of I14-I17, the respective coil is encased in a biocompatible material.

(I19) In some embodiments of any of I14-I18, the respective predefined frequency is at about 30 Hz to 300 Hz.

(I20) In some embodiments of any of I1-I19, the MCG subsystem is further configured to: generate motion artifacts-corrected biomagnetic field signals from the measured biomagnetic field signals in accordance with the determined location information of the one or more electrodes.

(I21) In some embodiments of I20, the MCG subsystem is further configured to: determine positional boundaries for an electrical source current of the heart of the living subject in accordance with the generated motion artifacts-corrected biomagnetic field signals.

(I22) In some embodiments of I20 or 121, the MCG subsystem is further configured to: determine a source location of the biomagnetic field signals in accordance with the generated motion artifacts-corrected biomagnetic field signals.

(I23) In some embodiments of any of I1-I22, the system is configured to determine a position of the living subject's heart relative to the plurality of magnetometers in accordance with the determined spatial relationship.

(I24) In some embodiments of any of I1-I23, the MCG subsystem and the ECG subsystem are temporally synchronized with each other.

(I25) In some embodiments of I24, the MCG subsystem and the ECG subsystem share a common clock.

(I26) In some embodiments of any of I1-I25, the electrode position detector is configured to determine the location information of the one or more electrodes that are mounted on the living subject using one or more cameras of the electrode position detector.

(I27) In some embodiments of any of I1-I26, each electrode of the one or more electrodes comprises a respective acoustic beacon of one or more acoustic beacons; and the electrode position detector is configured to determine the location information of the one or more electrodes by measuring acoustic signals emitted from the one or more acoustic beacons.

(J1) In accordance with some embodiments, a system comprises one or more electrodes configured to be mounted on a human subject, and each electrode is physically coupled to a respective electromagnetic transmitter. When the one or more electrodes are mounted on the human subject, the system is configured to: (1) measure, via a plurality of magnetometers that are communicatively couped to the system, first magnetic field signals from the one or more electromagnetic transmitters; (2) determine a spatial relationship between the plurality of magnetometers and the one or more electromagnetic transmitters based on the measured first magnetic signals; and (3) localize respective positions of the one or more electrodes.

(K1) In accordance with some embodiments, a method is performed at a system that includes a magnetocardiography (MCG) subsystem having a plurality of magnetometers, an electrocardiography (ECG) subsystem having a plurality of electrodes that are physically coupled to a human subject, and an electrode position detector. The method includes (1) measuring, via the plurality of electrodes of the ECG subsystem, ECG data associated with electrical activity of a heart of the human subject; (2) measuring, via the plurality of magnetometers of the MCG subsystem, biomagnetic field signals from the human subject; and (3) determining, via the electrode position detector, location information of the plurality of electrodes.

(K2) In some embodiments of K1, the location information of the plurality of electrodes comprises location information of the plurality of electrodes relative to the plurality of magnetometers.

(K3) In some embodiments of K1 or K2, (1) the MCG subsystem is the electrode position detector; (2) each electrode of the plurality of electrodes is physically coupled to a respective electromagnetic transmitter of one or more electromagnetic transmitters; and (3) determining, via the electrode position detector, the location information of the plurality of electrodes includes measuring, via the plurality magnetometers, first magnetic field signals emitted from the one or more electromagnetic transmitters.

(K4) In some embodiments of any of K1-K3, the method further includes correcting motion artifacts in the measured biomagnetic field signals in accordance with the determined location information of the plurality of electrodes to obtain motion artifacts-corrected biomagnetic field signals.

(K5) In some embodiments of K4, the method further includes: (1) extracting, from the measured ECG data, respective timings corresponding to individual heartbeats of the heart of the human subject; and (2) applying the extracted respective timings to the motion artifacts-corrected biomagnetic field signals to obtain to noise-reduced motion artifacts-corrected biomagnetic field signals.

(K6) In some embodiments of K5, the method further includes generating a magnetic field map based at least in part on the noise-reduced motion artifacts-corrected biomagnetic field signals, the magnetic field map visually depicting a magnetic field spatial distribution of the heart of the human subject.

(K7) In some embodiments of K5 or K6, the method further includes determining a magnetic source of the biomagnetic field signals in accordance with the noise-reduced motion artifacts-corrected biomagnetic field signals.

(K8) In some embodiments of K7. the method further includes determining the magnetic source of the biomagnetic field signals based on a magnetic field map generated from the noise-reduced motion artifacts-corrected biomagnetic field signals.

(K9) In some embodiments of K7 or K8, the method further includes estimating a cardiac electrophysiological source location in accordance with the determined magnetic source.

(K10) In some embodiments of any of K1-K9, the method further includes correcting motion artifacts in the ECG data in accordance with the determined location information of the plurality of electrodes, to obtain motion artifacts-corrected ECG data.

(K11) In some embodiments of K10, (1) the ECG data comprises time-series voltage measurements of the electrical activity of the heart; and (2) the method further includes: (3) in accordance with the determined location information of the plurality of electrodes and the motion artifacts-corrected ECG data: adjusting a scaling factor of a portion of the voltage measurements, corresponding to a first electrode of the plurality of electrodes.

(K12) In some embodiments of K10 or K11, the ECG data comprises time-series voltage measurements of the electrical activity of the heart; and the method further comprises, in accordance with the determined location information of the plurality of electrodes and the motion artifacts-corrected ECG data: (1) estimating a thickness of a first body portion of the human subject; and (2) adjust a scaling factor of a portion of the voltage measurements in accordance with the estimated thickness.

(K13) In some embodiments of any of K10-K12, the method further includes in accordance with the determined location information of the plurality of electrodes and the motion artifacts-corrected ECG data: estimating a location of the heart of the human subject.

(K14) In some embodiments of K13, The method of claim 41, the method further includes: determining a magnetic source of the biomagnetic field signals in accordance with the estimated location of the heart of the human subject.

(K15) In some embodiments of any of K1-K14, the method further includes: (1) determining, from the ECG data, a first portion of the ECG data corresponding to time intervals where the location information of the plurality of electrodes remain unchanged; and (2) estimating a location of the heart of the human subject based on the first portion of the ECG data.

(L1) In accordance with some embodiments, a system comprises a magnetocardiography (MCG) subsystem having a plurality of magnetometers, an electrocardiography (ECG) subsystem having a plurality of electrodes that are physically coupled to a human subject, an electrode position detector, one or more processors, memory; and one or more programs stored in the memory for execution by the one or more processors. The one or more programs comprise instructions for performing the method of any of K1-K15.

(M1) In accordance with some embodiments, a computer system comprises one or more processors, memory, and one or more programs stored in the memory configured for execution by the one or more processors. The one or more programs comprising instructions for performing the method of any of K1-K15.

(N1) In accordance with some embodiments, a computer readable storage medium stores computer-executable instructions that, when executed by one or more processors of the system of L1 or the computer system of M1, cause the system of L1 or the computer system of M1 to perform the method of any of K1-K15.

The methods disclosed herein comprise one or more steps or actions for achieving the described method. The method steps and/or actions may be interchanged with one another without departing from the scope of the claims. In other words, unless a specific order of steps or actions is required for proper operation of the method that is being described, the order and/or use of specific steps and/or actions may be modified without departing from the scope of the claims.

As used herein, the term “plurality” denotes two or more. For example, a plurality of components indicates two or more components. The term “determining” encompasses a wide variety of actions and, therefore, “determining” can include calculating, computing, processing, deriving, investigating, looking up (e.g., looking up in a table, a database or another data structure), ascertaining and the like. Also, “determining” can include receiving (e.g., receiving information), accessing (e.g., accessing data in a memory) and the like. Also, “determining” can include resolving, selecting, choosing, establishing and the like.

The phrase “based on” does not mean “based only on,” unless expressly specified otherwise. In other words, the phrase “based on” describes both “based only on” and “based at least on.”

As used herein, the term “exemplary” means “serving as an example, instance, or illustration,” and does not necessarily indicate any preference or superiority of the example over any other configurations or embodiments.

As used herein, the term “and/or” encompasses any combination of listed elements. For example, “A, B, and/or C” includes the following sets of elements: A only, B only, C only, A and B without C, A and C without B, B and C without A, and a combination of all three elements, A, B, and C.

The terminology used in the description of the invention herein is for the purpose of describing particular embodiments only and is not intended to be limiting of the invention. As used in the description of the invention and the appended claims, the singular forms “a,” “an,” and “the” are intended to include the plural forms as well, unless the context clearly indicates otherwise. It will also be understood that the term “and/or” as used herein refers to and encompasses any and all possible combinations of one or more of the associated listed items. It will be further understood that the terms “comprises” and/or “comprising,” when used in this specification, specify the presence of stated features, steps, operations, elements, and/or components, but do not preclude the presence or addition of one or more other features, steps, operations, elements, components, and/or groups thereof.

The foregoing description, for the purpose of explanation, has been described with reference to specific embodiments. However, the illustrative discussions above are not intended to be exhaustive or to limit the invention to the precise forms disclosed. Many modifications and variations are possible in view of the above teachings. The embodiments were chosen and described in order to best explain the principles of the invention and its practical applications, to thereby enable others skilled in the art to best utilize the invention and various embodiments with various modifications as are suited to the particular use contemplated.

What is claimed is:

1. A method for determining positional information of a human subject during a biomagnetic field scan, comprising:
at a computing device coupled to a plurality of magnetometers, one or more processors, and memory:
detecting, by the plurality of magnetometers:
first biomagnetic field signals from at least a portion of an organ of the human subject; and
second magnetic field signals from an electromagnetic transmitter positioned on a predetermined position of the human subject, wherein the plurality of magnetometers have a known position during the biomagnetic field scan;
determining a time-varying spatial relationship between the electromagnetic transmitter and the plurality of magnetometers based on the detected second magnetic field signals; and
correcting artifacts in the detected first biomagnetic signals in accordance with the determined spatial relationship.

2. The method of claim 1, wherein:
the second magnetic field signals are detected over a plurality of time points; and
the method includes:
determining, for each time point of the plurality of time points, a respective position and orientation of the electromagnetic transmitter on a coordinate system for the plurality of magnetometers.

3. The method of claim 1, further comprising:
determining a position of one or more body parts of the human subject relative to the plurality of magnetometers in accordance with the determined spatial relationship.

4. The method of claim 1, further comprising:
identifying, from the second magnetic field signals, one or more of:
a first subset of magnetic field signals having a first spectral characteristic; and
a second subset magnetic field signals having a second spectral characteristic that is distinct from the first spectral characteristic.

5. The method of claim 4, wherein the second spectral characteristic comprises a spectral contribution due to movement of a chest wall of the human subject.

6. The method of claim 1, wherein:
the organ of the human subject is a heart; and
the method further comprises determining seismocardiogram measurements of the human subject in accordance with the determined spatial relationship.

7. The method of claim 1, wherein the first biomagnetic field signals and the second magnetic field signals are detected synchronously by the plurality of magnetometers.

8. The method of claim 1, wherein the electromagnetic transmitter is positioned at an anatomical landmark position of the human subject, the anatomical position being a sternum, or a suprasternal notch, or a flank of the human subject.

9. The method of claim 1, wherein:
the electromagnetic transmitter comprises a coil; and
the second magnetic field signals are generated by providing an alternating current having a predefined amplitude and a predefined frequency through the coil.

10. The method of claim 9, wherein the predefined frequency of the alternating current is within a signal collection bandwidth of the plurality of magnetometers.

11. The method of claim 9, wherein a diameter of the coil is about 0.1 cm to 5 cm.

12. The method of claim 9, wherein the coil is encased in a biocompatible material.

13. The method of claim 9, wherein the alternating current comprises a frequency of about 70 Hz to 300 Hz.

14. The method of claim 9, wherein the alternating current through the coil is provided by a coil driving system that is coupled to the coil, and the method includes:
synchronizing the coil driving system with the plurality of magnetometers at a sampling rate of the magnetometers.

15. The method of claim 9, wherein detecting, by the plurality of magnetometers, the second magnetic field signals from the electromagnetic transmitter includes:
detecting, by each magnetometer of the plurality of magnetometers, a respective amplitude of the second magnetic field signals generated by the coil over time.

16. The method of claim 15, wherein determining the spatial relationship between the electromagnetic transmitter and the plurality of magnetometers based on the detected first biomagnetic field signals and second magnetic field signals includes:
determining a respective spatial relationship between the electromagnetic transmitter and each magnetometer of the plurality of magnetometers in accordance with the respective amplitude detected by the respective magnetometer.

17. The method of claim 15, wherein detecting, by each magnetometer of the plurality of magnetometers, the respective amplitude of the second magnetic field signals includes:
performing lock-in amplification at each magnetometer of the plurality of magnetometers to extract the respective amplitude.

18. The method of claim 1, wherein:
the electromagnetic transmitter is a first electromagnetic transmitter of a plurality of electromagnetic transmitters; and
each electromagnetic transmitter of the plurality of electromagnetic transmitters is positioned on a respective predetermined position of the human subject.

19. The method of claim 18, wherein each electromagnetic transmitter of the plurality of electromagnetic transmitters comprises a respective coil having a distinct driving current and a distinct predefined frequency.

20. The method of claim 18, wherein each electromagnetic transmitter of the plurality of electromagnetic transmitters is positioned at a respective predetermined position of the human subject via a respective electrocardiogram (ECG) electrode of a plurality of ECG electrodes, wherein the plurality of ECG electrodes are coupled to an ECG system.

21. The method of claim 20, further comprising:
measuring ECG cardiac activity via the plurality of ECG electrodes during the biomagnetic field scan.

22. A computer system coupled to a plurality of magnetometers, the computer system comprising:
one or more processors;
memory; and
one or more programs stored in the memory for execution by the one or more processors, the one or more programs comprising instructions for:
detecting, by the plurality of magnetometers:
first biomagnetic field signals from at least a portion of an organ of a human subject; and
second magnetic field signals from an electromagnetic transmitter positioned on a predetermined position of the human subject, wherein the plurality of magnetometers have a known position during the biomagnetic field scan;
determining a time-varying spatial relationship between the electromagnetic transmitter and the plurality of magnetometers based on the detected second magnetic field signals; and
correcting artifacts in the detected first biomagnetic signals in accordance with the determined spatial relationship.

23. A non-transitory computer readable storage medium storing computer-executable instructions that, when executed by one or more processors of a computer system that is coupled to a plurality of magnetometers, cause the computer system to:
detect, by the plurality of magnetometers:
first biomagnetic field signals from at least a portion of an organ of a human subject; and
second magnetic field signals from an electromagnetic transmitter positioned on a predetermined position of the human subject, wherein the plurality of magnetometers have a known position during the biomagnetic field scan;
determine a time-varying spatial relationship between the electromagnetic transmitter and the plurality of magnetometers based on the detected second magnetic field signals; and
correct artifacts in the detected first biomagnetic signals in accordance with the determined spatial relationship.
